(19) United States
(12) Patent Application Publication
(10) Pub. No.: US 2009/0148447 A1
(43) Pub. Date: Jun. 11, 2009

(54) Binding Peptides Having a C-terminally Disposed Specific Binding Domain

(75) Inventors: Jeffrey A. Ledbetter, Shoreline, WA (US); William Brady, Bothell, WA (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: TRUBION PHARMACEUTICALS, INC., Seattle, WA (US)

(21) Appl. No.: 12/168,875

(22) Filed: Jul. 7, 2008

Related U.S. Application Data
(60) Provisional application No. 60/948,397, filed on Jul. 6, 2007.

(51) Int. Cl. A61K 39/395; C07K 16/18; C12P 21/02

(52) U.S. Cl. 424/134.1; 530/387.3; 435/69.6

(57) ABSTRACT

Specific binding peptides having a general schematized structure of an optional N-terminal hinge region joined to an immunoglobulin-derived constant sub-region comprising a C_{H2} region and a C_{H3} region, followed by a PIMS linker peptide and at least one specific binding domain are provided, along with encoding nucleic acids, vectors and host cells. Also provided are methods for making such peptides and methods for using such peptides to treat or prevent a variety of diseases, disorders or conditions, as well as to ameliorate at least one symptom associated with such a disease, disorder or condition.

### FIELD

The invention relates generally to the field of specific binding molecules and therapeutic applications thereof.

### BACKGROUND

In a healthy mammal, the immune system protects the body from damage from foreign substances and pathogens. In some instances though, the immune system goes awry, producing traumatic insult and/or disease. For example, B-cells can produce antibodies that recognize self-proteins rather than foreign proteins, leading to the production of the autoantibodies characteristic of autoimmune diseases such as lupus erythematosus, rheumatoid arthritis, and the like. In other instances, the typically beneficial effect of the immune system in combating foreign materials is counterproductive, such as following organ transplantation. The power of the mammalian immune system, and in particular the human immune system, has been recognized and efforts have been made to control the system to avoid or ameliorate the deleterious consequences to health that result either from normal functioning of the immune system in an abnormal environment (e.g., organ transplantation) or from abnormal functioning of the immune system in an otherwise apparently normal environment (e.g., autoimmune disease progression). Additionally, efforts have been made to exploit the immune system to provide a number of target-specific diagnostic and therapeutic methodologies, relying on the capacity of antibodies to specifically recognize and bind antigenic targets with specificity.

One way in which the immune system protects the body is by production of specialized cells called B lymphocytes or B-cells. B-cells produce antibodies that bind to, and in some cases mediate destruction of, a foreign substance or pathogen. In some instances though, the human immune system, and specifically the B lymphocytes of the human immune system, go awry and disease results. There are numerous cancers that involve uncontrolled proliferation of B-cells. There are also numerous autoimmune diseases that involve B-cell production of antibodies that, instead of binding to foreign substances and pathogens, bind to parts of the body. In addition, there are numerous autoimmune and inflammatory diseases that involve B-cells in their pathology, for example, through inappropriate B-cell antigen presentation to T-cells or through other pathways involving B-cells. For example, autoimmune-prone mice deficient in B-cells do not develop autoimmune kidney disease, vasculitis or autoantibodies. (Shlomchik et al., J. Exp. Med. 1994, 180:1295-306). Interestingly, these same autoimmune-prone mice which possess B-cells but are deficient in immunoglobulin production, do develop autoimmune diseases when induced experimentally (Chan et al., J. Exp. Med. 1999, 189:1639-48), indicating that B-cells play an integral role in development of autoimmune disease.

B-cells can be identified by molecules on their cell surface. CD20 was the first human B-cell lineage-specific surface molecule identified by a monoclonal antibody. It is a non-glycosylated, hydrophobic 35 kDa B-cell transmembrane phosphoprotein that has both its amino and carboxy ends situated inside the cell. Einfeld et al., EMBO J. 1988, 7:711-17. CD20 is expressed by all normal mature B-cells, but is not expressed by precursor B-cells or plasma cells. Natural ligands for CD20 have not been identified, and the function of CD20 in B-cell biology is still incompletely understood.

Another B-cell lineage-specific cell surface molecule is CD37. CD37 is a heavily glycosylated 40-52 kDa protein that belongs to the tetraspanin transmembrane family of cell surface antigens. It traverses the cell membrane four times forming two extracellular loops and exposing its amino and carboxy ends to the cytoplasm. CD37 is highly expressed on normal antibody-producing (sIg+)B-cells, but is not expressed on pre-B-cells or plasma cells. The expression of CD37 on resting and activated T cells, monocytes and granulocytes is low and there is no detectable CD37 expression on NK cells, platelets or erythrocytes. See, Belov et al., Cancer Res., 61(11):4483-4489 (2001); Schwartz-Albiez et al., J. Immunol., 140(3): 905-914 (1988); and Link et al., J. Immunol., 137(9): 3013-3018 (1988). Besides normal B-cells, almost all malignancies of B-cell origin are positive for CD37 expression, including CLL, NHL, and hairy cell leukemia (Moore, et al. 1987; Merson and Brochier 1988; Faure, et al. 1990). CD37 participates in regulation of B-cell function, since mice lacking CD37 were found to have low levels of serum IgG1 and to be impaired in their humoral response to viral antigens and model antigens. It appears to act as a nonclassical costimulatory molecule or by directly influencing antigen presentation via complex formation with MHC class II molecules. See Knobeloch et al., Mol. Cell. Biol., 20(15):5363-5369 (2000).

Research and drug development has occurred based on the concept that B-cell lineage-specific cell surface molecules such as CD37 and CD20 can themselves be targets for antibodies that would bind to, and mediate destruction of, cancerous and autoimmune disease-causing B-cells that have CD37 and CD20 on their surfaces. Termed “immunotherapy,” antibodies made (or based on antibodies made) in a non-human animal that bind to CD37 or CD20 were given to a patient to deplete cancerous or autoimmune disease-causing B-cells.

Monoclonal antibody technology and genetic engineering methods have facilitated development of immunoglobulin molecules for diagnosis and treatment of human diseases. The domain structure of immunoglobulins is amenable to engineering, in that the antigen binding domains and the domains conferring effector functions may be exchanged between immunoglobulin classes and subclasses. Immunoglobulin structure and function are reviewed, for example, in Harlow et al., Eds., Antibodies: A Laboratory Manual, Chapter 14, Cold Spring Harbor Laboratory, Cold Spring Harbor (1988). An extensive introduction as well as detailed information about all aspects of recombinant antibody technology can be found in the textbook “Recombinant Antibodies” (John Wiley & Sons, NY, 1999). A comprehensive collection of detailed antibody engineering lab Protocols can be found in R. Kontermann and S. Dübel (eds.), “The Antibody Engineering Lab Manual” (Springer Verlag, Heidelberg/New York, 2000).

An immunoglobulin molecule (abbreviated Ig), is a multimeric protein, typically composed of two identical light chain polypeptides and two identical heavy chain polypeptides (H₂L₂) that are joined into a macromolecular complex by interchain disulfide bonds, i.e., covalent bonds between the sulfhydryl groups of neighboring cysteine residues. Five human immunoglobulin classes are defined on the basis of their heavy chain composition, and are named IgG, IgM, IgA, IgE, and IgD. The IgG-class and IgA-class antibodies are further divided into subclasses, namely, IgG1, IgG2, IgG3, and IgG4, and IgA1 and IgA2, respectively. Intrachain disulfide bonds join different areas of the same polypeptide chain, which results in the formation of loops that, along with adjacent amino acids, constitute the immunoglobulin domains. At the amino-terminal portion, each light chain and each heavy chain has a single variable region that shows considerable variation in amino acid composition from one antibody to another. The light chain variable region, V_{L}, has a single antigen-binding domain and associates with the variable region of a heavy chain, V_{H} (also containing a single antigen-binding domain), to form the antigen binding site of the immunoglobulin, the Fv.

In addition to variable regions, each of the full-length antibody chains has a constant region containing one or more domains. Light chains have a constant region containing a single domain. Thus, light chains have one variable domain and one constant domain. Heavy chains have a constant region containing several domains. The heavy chains in IgG, IgA, and IgD antibodies have three domains, which are designated C_{H1}, C_{H2}, and C_{H3}; the heavy chains in IgM and IgE antibodies have four domains, C_{H1}, C_{H2}, C_{H3} and C_{H4}. Thus, heavy chains have one variable domain and three or four constant domains. Noteworthy is the invariant organization of these domains in all known species, with the constant regions, containing one or more domains, being located at or near the C-terminus of both the light and heavy chains of immunoglobulin molecules, with the variable domains located towards the N-termini of the light and heavy chains. Immunoglobulin structure and function are reviewed, for example, in Harlow et al., Eds., Antibodies: A Laboratory Manual, Chapter 14, Cold Spring Harbor Laboratory, Cold Spring Harbor (1988).

The heavy chains of immunoglobulins can also be divided into three functional regions: the Fd region (a fragment comprising V_{H} and C_{H1}, i.e., the two N-terminal domains of the heavy chain), the hinge region, and the Fc region (the “fragment crystallizable” region). The Fc region contains the domains that interact with immunoglobulin receptors on cells and with the initial elements of the complement cascade. Thus, the Fc region or fragment is generally considered responsible for the effector functions of an immunoglobulin, such as ADCC (antibody-dependent cell-mediated cytotoxicity), CDC (complement-dependent cytotoxicity) and complement fixation, binding to Fc receptors, greater half-life in vivo relative to a polypeptide lacking an Fc region, protein A binding, and perhaps even placental transfer. Capon et al., Nature, 337: 525-531, (1989). Further, a polypeptide containing an Fc region allows for dimerization/multimerization of the polypeptide. These terms are also used for analogous regions of the other immunoglobulins.

Although all of the human immunoglobulin isotypes contain a recognizable structure in common, each isotype exhibits a distinct pattern of effector function. IgG, by way of nonexhaustive example, neutralizes toxins and viruses, opsonizes, fixes complement (CDC) and participates in ADCC. IgM, in contrast, neutralizes blood-borne pathogens and participates in opsonization. IgA, when associated with its secretory piece, is secreted and provides a primary defense to microbial infection via the mucosa; it also neutralizes toxins and supports opsonization. IgE mediates inflammatory responses, being centrally involved in the recruitment of other cells needed to mount a full response. IgD is known to provide an immunoregulatory function, controlling the activation of B cells. These characterizations of isotype effector functions provide a non-comprehensive illustration of the differences that can be found among human isotypes.

The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. The four IgG subclasses also differ from each other with respect to their effector functions. This difference is related to differences in structure, including differences with respect to the interaction between the variable region, Fab fragments, and the constant Fc fragment.

According to crystallographic studies, the immunoglobulin hinge region can be further subdivided functionally into three regions: the upper hinge region, the core region, and the lower hinge region. Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of human IgG1 contains the sequence Cys-Pro-Pro-Cys which, when dimerized by disulfide bond formation, results in a cyclic octapeptide believed to act as a pivot, thus conferring flexibility. The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin.

Conformational changes permitted by the structure and flexibility of the immunoglobulin hinge region polypeptide sequence may also affect the effector functions of the Fc portion of the antibody. Three general categories of effector functions associated with the Fc region include (1) activation of the classical complement cascade, (2) interaction with effector cells, and (3) compartmentalization of immunoglobulins. The different human IgG subclasses vary in the relative efficacies with which they fix complement, or activate and amplify the steps of the complement cascade. See, e.g., Kirschfink, 2001 Immunol. Rev. 180:177; Chakraborti et al., 2000 Cell Signal 12:607; Kohl et al., 1999 Mol. Immunol. 36:893; Marsh et al., 1999 Curr. Opin. Nephrol. Hypertens. 8:557; Speth et al., 1999 Wien Klin. Wochenschr. 111:378.

Exceptions to the H₂L₂ structure of conventional antibodies occur in some isotypes of the immunoglobulins found in camelids (camels, dromedaries and llamas; Hamers-Casterman et al., 1993 Nature 363:446; Nguyen et al., 1998 J. Mol. Biol. 275:413), nurse sharks (Roux et al., 1998 Proc. Nat. Acad. Sci. USA 95:11804), and in the spotted ratfish (Nguyen, et al., 2002 Immunogenetics 54(1):39-47). These antibodies can apparently form antigen-binding regions using only heavy chain variable region, i.e., these functional antibodies are homodimers of heavy chains only (referred to as “heavy-chain antibodies” or “HCAbs”). Despite the advantages of antibody technology in disease diagnosis and treatment, there are some disadvantageous aspects of developing whole-antibody technologies as diagnostic and/or therapeutic reagents. Whole antibodies are large protein structures exemplified by the heterotetrameric structure of the IgG isotype, containing two light and two heavy chains. Such large molecules are sterically hindered in certain applications. For example, in treatments of solid tumors, whole antibodies do not readily penetrate the interior of the tumor. Moreover, the relatively large size of whole antibodies presents a challenge to ensure that the in vivo administration of such molecules does not induce an immune response. Further, generation of active antibody molecules typically involves the culturing of recombinant eukaryotic cells capable of providing appropriate post-translational processing of the nascent antibody molecules, and such cells can be difficult to culture and difficult to induce in a manner that provides commercially useful yields of active antibody.

Recently, smaller immunoglobulin molecules have been constructed to overcome problems associated with whole immunoglobulin methodologies. A single-chain variable antibody fragment (scFv) comprises an antibody heavy chain variable domain joined via a short peptide to an antibody light chain variable domain (Huston et al., Proc. Natl. Acad. Sci. USA, 1988, 85: 5879-83). Because of the small size of scFv molecules, they exhibit more effective penetration into tissues than whole immunoglobulin. An anti-tumor scFv showed more rapid tumor penetration and more even distribution through the tumor mass than the corresponding chimeric antibody (Yokota et al., Cancer Res. 1992, 52:3402-08).

Despite the advantages that scFv molecules bring to serotherapy, several drawbacks to this therapeutic approach exist. An scFv is rapidly cleared from the circulation, which may reduce toxic effects in normal cells, but such rapid clearance impedes delivery of a minimum effective dose to the target tissue. Manufacturing adequate amounts of scFv for administration to patients has been challenging due to difficulties in expression and isolation of scFv that adversely affect the yield. Another disadvantage to using scFv for therapy is the lack of effector function. Alternatively, it has been proposed that fusion of an scFv to another molecule, such as a toxin, could take advantage of the specific antigen-binding activity and the small size of an scFv to deliver the toxin to a target tissue, but dosing with such conjugates or chimeras can be limited by excessive and/or non-specific toxicity due to the toxin moiety of such preparations. In addition, immunotoxins are themselves highly immunogenic upon administration to a host, and host antibodies generated against the immunotoxin limit potential usefulness for repeated therapeutic treatments of an individual.

Nonsurgical cancer therapy, such as external irradiation and chemotherapy, can suffer from limited efficacy because of toxic effects on normal tissues and cells, due to the lack of specificity these treatments exhibit towards cancer cells. To overcome this limitation, targeted treatment methodologies have been developed to increase the specificity of the treatment for the cells and tissues in need thereof. An example of such a targeted methodology for in vivo use is the administration of antibody conjugates, with the antibody designed to specifically recognize a marker associated with a cell or tissue in need of treatment, and the antibody being conjugated to a therapeutic agent, such as a toxin in the case of cancer treatment. Antibodies, as systemic agents, circulate to sensitive and undesirable body compartments, such as the bone marrow. In acute radiation injury, destruction of lymphoid and hematopoietic compartments is a major factor in the development of septicemia and subsequent death. Moreover, antibodies are large, globular proteins that can exhibit poor penetration of tissues in need of treatment.

Human patients and non-human subjects suffering from a variety of end-stage disease processes frequently require organ transplantation. Organ transplantation, however, must contend with the untoward immune response of the recipient and guard against immunological rejection of the transplanted organ by depressing the recipient's cellular immune response to the foreign organ with cytotoxic agents which affect the lymphoid and other parts of the hematopoietic system. Graft acceptance is limited by the tolerance of the recipient to these cytotoxic chemicals, many of which are similar to the anticancer (antiproliferative) agents. Likewise, when using cytotoxic antimicrobial agents, particularly antiviral drugs, or when using cytotoxic drugs for autoimmune disease therapy, e.g., in treatment of systemic lupus erythematosis, a serious limitation is the toxic effects of the therapeutic agents on the bone marrow and the hematopoietic cells of the body.

Use of targeted therapies, such as targeted antibody conjugate therapy, is designed to localize a maximum quantity of the therapeutic agent at the site of desired action as possible, and the success of such therapies is revealed by the relatively high signal-to-background ratio of therapeutic agent. Examples of targeted antibodies include diagnostic or therapeutic agent conjugates of antibody or antibody fragments, cell- or tissue-specific peptides, and hormones and other receptor-binding molecules. For example, antibodies against different determinants associated with pathological and normal cells, as well as associated with pathogenic microorganisms, have been used for the detection and treatment of a wide variety of pathological conditions or lesions. In these methods, the targeting antibody is directly conjugated to an appropriate detecting or therapeutic agent as described, for example, in Hansen et al., U.S. Pat. No. 3,927,193 and Goldenberg, U.S. Pat. Nos. 4,331,647, 4,348,376, 4,361,544, 4,468,457, 4,444,744, 4,460,459, 4,460,561, 4,624,846 and 4,818,709.

One problem encountered in direct targeting methods, i.e., in methods wherein the diagnostic or therapeutic agent (the “active agent”) is conjugated directly to the targeting moiety, is that a relatively small fraction of the conjugate actually binds to the target site, while the majority of conjugate remains in circulation and compromises in one way or another the function of the targeted conjugate. To ensure maximal localization of the active agent, an excess of the targeted conjugate is typically administered, ensuring that some conjugate will remain unbound and contribute to background levels of the active agent.

Complement-dependent cytotoxicity (CDC) is believed to be a significant mechanism for clearance of specific target cells such as tumor cells. CDC is a series of events that consists of a collection of enzymes that become activated by each other in a cascade fashion. Complement has an important role in clearing antigen, accomplished by its four major functions: (1) local vasodilation; (2) attraction of immune cells, especially phagocytes (chemotaxis); (3) tagging of foreign organisms for phagocytosis (opsonization); and (4) destruction of invading organisms by the membrane attack complex (MAC attack). The central molecule is the C3 protein. It is an enzyme that is split into two fragments by components of either the classical pathway or the alternative pathway. The classical pathway is induced by antibodies, especially IgG and IgM, while the alternative pathway is nonspecifically stimulated by bacterial products like lipopolysaccharide (LPS). Briefly, the products of the C3 split include a small peptide C3a which is chemotactic for phagocytic immune cells and results in local vasodilation by causing the release of C5a fragment from C5. The other part of C3, C3b, coats antigens on the surface of foreign organisms and acts to opsonize the organism for destruction. C3b also reacts with other components of the complement system to form an MAC consisting of C5b, C6, C7, C8 and C9.

There are problems associated with the use of antibodies in human therapy because the response of the immune system to any antigen, even the simplest, is “polyclonal,” i.e., the system manufactures antibodies of a great range of structures both in their binding regions as well as in their effector regions. Two approaches have been used in an attempt to reduce the problem of immunogenic antibodies. The first is the production of chimeric antibodies in which the antigen-binding part (variable regions) of a mouse monoclonal antibody is fused to the effector part (constant region) of a human antibody. In a second approach, antibodies have been altered through a technique known as complementarity determining region (CDR) grafting or “humanization.” This process has been further improved to include changes referred to as “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., In: Metcalf B W, Dalton B J, eds. Cellular adhesion: molecular definition to therapeutic potential. New York: Plenum Press, 1994:291-312).

A variety of antibody technologies have received attention in the effort to develop and market more effective therapeutics and palliatives. Unfortunately, problems continue to compromise the promise of each of these therapies. For example, the majority of cancer patients treated with rituximab relapse, generally within about 6-12 months, and fatal infusion reactions within 24 hours of rituximab infusion have been reported. Trastuzumab administration can result in the development of ventricular dysfunction, congestive heart failure, and severe hypersensitivity reactions (including anaphylaxis), infusion reactions, and pulmonary events. Daclizumab immunosuppressive therapy poses an increased risk for developing lymphoproliferative disorders and opportunistic infections. Death from liver failure, arising from severe hepatotoxicity, and from veno-occlusive disease (VOD), has been reported in patients who received gemtuzumab. Hepatotoxicity was also reported in patients receiving alemtuzumab.

Cancer includes a broad range of diseases, affecting approximately one in four individuals worldwide. Rapid and unregulated proliferation of malignant cells is a hallmark of many types of cancer, including hematological malignancies. Although patients with a hematologic malignant condition have benefited from advances in cancer therapy in the past two decades, Multani et al., 1998 J. Clin. Oncology 16:3691-3710, and remission times have increased, most patients still relapse and succumb to their disease. Barriers to cure with cytotoxic drugs include, for example, tumor cell resistance and the high toxicity of chemotherapy, which prevents optimal dosing in many patients.

Treatment of patients with low grade or follicular B cell lymphoma using a chimeric CD20 monoclonal antibody has been reported to induce partial or complete responses in patients. McLaughlin et al., 1996 Blood 88:90a (abstract, suppl. 1); Maloney et al., 1997 Blood 90:2188-95. However, as noted above, tumor relapse commonly occurs within six months to one year. Further improvements in serotherapy are needed to induce more durable responses, for example, in low grade B cell lymphoma, and to allow effective treatment of high grade lymphoma and other B cell diseases.

Autoimmune diseases include autoimmune thyroid diseases, which include Graves' disease and Hashimoto's thyroiditis. Another autoimmune disease is rheumatoid arthritis (RA), which is a chronic disease characterized by inflammation of the joints, leading to swelling, pain, and loss of function. RA is caused by a combination of events including an initial infection or injury, an abnormal immune response, and genetic factors. While autoreactive T cells and B cells are present in RA, the detection of high levels of antibodies that collect in the joints, called rheumatoid factor, is used in the diagnosis of RA. Current therapy for RA includes many medications for managing pain and slowing the progression of the disease. Systemic Lupus Erythematosus (SLE) is an autoimmune disease caused by recurrent injuries to blood vessels in multiple organs, including the kidney, skin, and joints. In patients with SLE, a faulty interaction between T cells and B cells results in the production of autoantibodies that attack the cell nucleus.

There are several other recognized autoimmune diseases. Sjogren's syndrome is an autoimmune disease characterized by destruction of the body's moisture-producing glands. Immune thrombocytopenic purpura (ITP) is caused by autoantibodies that bind to blood platelets and cause their destruction. Multiple sclerosis (MS) is also an autoimmune disease. It is characterized by inflammation of the central nervous system and destruction of myelin, which insulates nerve cell fibers in the brain, spinal cord, and body. Myasthenia Gravis (MG) is a chronic autoimmune neuromuscular disorder that is characterized by weakness of the voluntary muscle groups. MG is caused by autoantibodies that bind to acetylcholine receptors expressed at neuromuscular junctions. The autoantibodies reduce or block acetylcholine receptors, preventing the transmission of signals from nerves to muscles. Psoriasis affects approximately five million people, and is characterized by autoimmune inflammation in the skin. Scleroderma is a chronic autoimmune disease of the connective tissue that is also known as systemic sclerosis. Scleroderma is characterized by an overproduction of collagen, resulting in a thickening of the skin.

Apparent from the foregoing discussion are needs for improved compositions and methods to treat, ameliorate or prevent a variety of diseases, disorders and conditions, including cancer, inflammation and autoimmune diseases.

### SUMMARY

The invention satisfies at least one of the aforementioned needs in the art by providing proteins containing a constant sub-region derived from an antibody molecule joined through a linker (PIMS linker) that has an amino acid sequence derived from an antibody hinge region, or a region linking a binding domain, or a region linking a binding domain to a cell surface transmembrane region or membrane anchor, to at least one specific binding domain, as well as nucleic acids encoding such proteins, and to production, diagnostic and therapeutic uses of such proteins and nucleic acids. The proteins of the invention are referred to herein as PIMS molecules. The PIMS linker has an amino acid sequence derived from an antibody hinge region, or a region linking a binding domain, or a region linking a binding domain to a cell surface transmembrane region or membrane anchor. In some embodiments, the linker has at least one cysteine residue capable of participating in at least one disulfide bond under standard peptide conditions. In some embodiments, a PIMS molecule further comprises an N-terminal domain derived from an antibody hinge region that may be the same as, or different than, the PIMS linker joining the constant sub-region and the specific binding domain(s). Typical thinking had been that the placement of a constant region derived from an antibody in the interior of a protein or at the N-terminus thereof would interfere with effector function, by analogy to the conventional placement of constant regions of antibodies at the carboxy termini of antibody chains. Placement of a constant sub-region at the N-terminus or in the interior of a polypeptide or protein chain in accordance with the invention, however, resulted in proteins exhibiting effector function and specific binding capacities relatively unencumbered by steric hindrances. As will be apparent to one of skill in the art upon consideration of this disclosure, proteins of such structure, and the nucleic acids encoding those proteins, will find a wide variety of applications, including medical and veterinary applications.

In one aspect, the invention provides a preferred form of a specific binding protein comprising a constant sub-region comprising part or all of a C_{H2} domain and part or all of a C_{H3} domain; a PIMS linker region disposed C-terminal to the constant sub-region; and at least one specific binding domain comprising part or all of a V_{L} domain and part or all of a V_{H} domain and exhibiting the capacity to specifically bind a binding partner, the specific binding domain(s) disposed C-terminal to the PIMS linker, wherein the specific binding protein specifically binds at least one target and exhibits at least one effector function of an antibody molecule. Thus, in preferred PIMS protein molecules, the PIMS linker, and therefore the constant sub-region, are disposed N-terminal to each specific binding domain of the molecule. In some embodiments, at least one of the C_{H2} domain and the C_{H3} domain is a complete antibody domain. Suitable specific binding proteins of the invention include proteins with an antibody domain that is selected from the group consisting of IgG (IgG1, IgG2, IgG3, IgG4), IgE, IgD, IgA (IgA1, IgA2), and IgM antibody domains. Such molecules include specific binding proteins wherein the C_{H2} domain comprises a sequence set forth in SEQ ID NO:377 and/or the C_{H3} domain comprises a sequence set forth in SEQ ID NO:379. Exemplary effector functions provided by the constant sub-region include antibody-dependent cellular cytotoxicity and/or complement-mediated cytotoxicity.

A PIMS linker region suitable for use in the PIMS molecules according to the invention may be selected from the group consisting of an antibody hinge region, a stalk region of a Type II C-lectin molecule (e.g., a CD72 stalk region), an NKG2a region, an NKG2A C18S region and variants thereof. For example, a suitable PIMS linker includes an antibody hinge region selected from the group consisting of IgG, IgA, IgD and IgE hinges and variants thereof. For example, the PIMS linker may be an antibody hinge region selected from the group consisting of human IgG1, human IgG2, human IgG3, and human IgG4, and variants thereof. In some embodiments, the PIMS linker region has a single cysteine residue for formation of an interchain disulfide bond. In other embodiments, the PIMS linker has two cysteine residues for formation of interchain disulfide bonds. PIMS linker regions contemplated for use in the PIMS molecules include a hinge region comprising a sequence selected from the group consisting of SEQ ID NO:61 to SEQ ID NO:118.

Also contemplated as PIMS linker regions of PIMS molecules are residues 268-281 of SEQ ID NO:2, residues 268-282 of SEQ ID NO:3, residues 268-282 of SEQ ID NO:5, residues 268-282 of SEQ ID NO:6, residues 268-282 of SEQ ID NO:8, residues 268-281 of SEQ ID NO:9, residues 268-282 of SEQ ID NO:11, residues 268-282 of SEQ ID NO:12, residues 268-281 of SEQ ID NO:14, residues 268-282 of SEQ ID NO:16, residues 268-282 of SEQ ID NO:18, residues 268-282 of SEQ ID NO:20, residues 268-282 of SEQ ID NO:22, residues 268-282 of SEQ ID NO:24, residues 268-282 of SEQ ID NO:26, residues 268-282 of SEQ ID NO:28, residues 268-282 of SEQ ID NO:30, residues 279-293 of SEQ ID NO:32, residues 274-288 of SEQ ID NO:34, residues 274-288 of SEQ ID NO:34, residues 261-275 of SEQ ID NO:36, residues 268-283 of SEQ ID NO:38, residues 268-282 of SEQ ID NO:40, residues 270-284 of SEQ ID NO:42, residues 265-279 of SEQ ID NO:44, residues 265-279 of SEQ ID NO:46, residues 265-279 of SEQ ID NO:48, residues 265-279 of SEQ ID NO:50, residues 265-279 of SEQ ID NO:52, residues 265-279 of SEQ ID NO:54, residues 265-279 of SEQ ID NO:56, residues 265-279 of SEQ ID NO:58, residues 268-282 of SEQ ID NO:60, residues 24-38 of SEQ ID NO:359, residues 24-38 of SEQ ID NO:361, residues 24-38 of SEQ ID NO:363, residues 24-38 of SEQ ID NO:365, residues 24-38 of SEQ ID NO:367, residues 24-38 of SEQ ID NO:369, residues 23-37 of SEQ ID NO:371, residues 23-37 of SEQ ID NO:373 and residues 23-37 of SEQ ID NO:375. In addition, any sequence of amino acids identified in the sequence listing as providing the sequence of a hinge region is contemplated for use as a PIMS linker in the PIMS molecules according to the invention. More generally, a PIMS linker may be a hinge-like peptide domain having at least one free cysteine capable of participating in an interchain disulfide bond. Additionally, a PIMS linker is a stalk region of a Type II C-lectin molecule.

In some embodiments, the specific binding protein or PIMS protein (or polypeptide) specifically binds to one of a wide variety of targets, including but not limited to CD3, CD19, CD20, CD28, CD37 and DR. Exemplary PIMS molecules or proteins are specific binding proteins selected from the group consisting of W0001 (SEQ ID NO:359 encoded by, e.g., SEQ ID NO:358), W0002 (SEQ ID NO:361 encoded by, e.g., SEQ ID NO:360), W0003 (SEQ ID NO:363 encoded by, e.g., SEQ ID NO:362), W0004 (SEQ ID NO:365 encoded by, e.g., SEQ ID NO:364), W0005 (SEQ ID NO:367 encoded by, e.g., SEQ ID NO:366), W0006 (SEQ ID NO:369 encoded by, e.g., SEQ ID NO:368), W0007 (SEQ ID NO:371 encoded by, e.g., SEQ ID NO:370), W0008 (SEQ ID NO:373 encoded by, e.g., SEQ ID NO:372), W0009 (SEQ ID NO:375 encoded by, e.g., SEQ ID NO:374), W0011 (SEQ ID NO:391 encoded by, e.g., SEQ ID NO:390), W0012 (SEQ ID NO:405 encoded by, e.g., SEQ ID NO:404), W0023 (SEQ ID NO:407 encoded by, e.g., SEQ ID NO:406), W0024 (SEQ ID NO:409 encoded by, e.g., SEQ ID NO:408), W0025 (SEQ ID NO:411 encoded by, e.g., SEQ ID NO:410), W0028 (SEQ ID NO:487 encoded by, e.g., SEQ ID NO:486), W0029 (SEQ ID NO:481 encoded by, e.g., SEQ ID NO:480), W0030 (SEQ ID NO:483 encoded by, e.g., SEQ ID NO:482), W0031 (SEQ ID NO:485 encoded by, e.g., SEQ ID NO:484), W0035 (SEQ ID NO:490 encoded by, e.g., SEQ ID NO:489), W0036 (SEQ ID NO:492 encoded by, e.g., SEQ ID NO:491), W0041 (SEQ ID NO:498 encoded by, e.g., SEQ ID NO:497), W0042 (SEQ ID NO:500 encoded by, e.g., SEQ ID NO:499), W0044 (SEQ ID NO:504 encoded by, e.g., SEQ ID NO:503), W0045 (SEQ ID NO:506 encoded by, e.g., SEQ ID NO:505), W0050 (SEQ ID NO:453 encoded by, e.g., SEQ ID NO:452), W0051 (SEQ ID NO:455 encoded by, e.g., SEQ ID NO:454), W0052 (SEQ ID NO:457 encoded by, e.g., SEQ ID NO:456), W0053 (SEQ ID NO:459 encoded by, e.g., SEQ ID NO:458), W0055 (SEQ ID NO:511 encoded by, e.g., SEQ ID NO:510), W0056 (SEQ ID NO:494 encoded by, e.g., SEQ ID NO:493), W0057 (SEQ ID NO:508 encoded by, e.g., SEQ ID NO:507), W0083 (SEQ ID NO:461 encoded by, e.g., SEQ ID NO:460), W0087 (SEQ ID NO:496 encoded by, e.g., SEQ ID NO:495), W0094 (SEQ ID NO:445 encoded by, e.g., SEQ ID NO:444), W0095 (SEQ ID NO:447 encoded by, e.g., SEQ ID NO:446), W0096 (SEQ ID NO:449 encoded by, e.g., SEQ ID NO:448), W0097 (SEQ ID NO:451 encoded by, e.g., SEQ ID NO:450), DNE090 (SEQ ID NO:393 encoded by, e.g., SEQ ID NO:392), DNE091 (SEQ ID NO:395 encoded by, e.g., SEQ ID NO:394), DNE092 (SEQ ID NO:397 encoded by, e.g., SEQ ID NO:396), DNE093 (SEQ ID NO:399 encoded by, e.g., SEQ ID NO:398), DNE094 (SEQ ID NO:401 encoded by, e.g., SEQ ID NO:400) and DNE095 (SEQ ID NO:403 encoded by, e.g., SEQ ID NO:402).

Contemplated for use in a PIMS molecule is a PIMS linker comprising the amino acid sequence set forth in any one of SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:172, SEQ ID NO:174, SEQ ID NO:176, SEQ ID NO:180, SEQ ID NO:182, SEQ ID NO:184, SEQ ID NO:186, SEQ ID NO:188, SEQ ID NO:190, SEQ ID NO:192, SEQ ID NO:194, SEQ ID NO:196, SEQ ID NO:198, SEQ ID NO:200, SEQ ID NO:202, SEQ ID NO:204, SEQ ID NO:206, SEQ ID NO:208, SEQ ID NO:210, SEQ ID NO:212, SEQ ID NO:214, SEQ ID NO:216, SEQ ID NO:222, SEQ ID NO:230, SEQ ID NO:238, SEQ ID NO:240, SEQ ID NO:242, SEQ ID NO:244, SEQ ID NO:246, SEQ ID NO:248, SEQ ID NO:541, SEQ ID NO:542, SEQ ID NO:543, SEQ ID NO:544, SEQ ID NO:545, SEQ ID NO:546, SEQ ID NO:547 and SEQ ID NO:548. Also contemplated are specific binding proteins wherein the V_{L} domain and the V_{H} domain are separated by an interdomain linker. In such embodiments, the interdomain linker may exhibit the structure of (Gly₄Ser)ₙ, where n is preferably 1-5. Exemplary interdomain linkers suitable for use in PIMS molecules include, but are not limited to, H11 (SEQ ID NO:544), H12 (SEQ ID NO:545), H17 (SEQ ID NO:184), H45 (SEQ ID NO:240) and H46 (SEQ ID NO:242), as well as (Gly4Ser)ₙ-based linkers such as those disclosed in SEQ ID NO:243, SEQ ID NO:245, SEQ ID NO:247, SEQ ID NO:248, SEQ ID NO:539 and SEQ ID NO:540. In addition, any sequence of amino acids identified in the sequence listing as providing the sequence of a linker is contemplated for use in the PIMS molecules according to the invention.

In some embodiments, the specific binding protein has at least one of the V_{L} domain and the V_{H} domain that comprises a sequence selected from the group consisting of residues 23-128 of SEQ ID NO:2, residues 145-265 of SEQ ID NO:2, residues 520-640 of SEQ ID NO:2, residues 661-772 of SEQ ID NO:2, residues 508-629 of SEQ ID NO:28, residues 647-754 of SEQ ID NO:28, residues 508-629 of SEQ ID NO:30, residues 652-759 of SEQ ID NO:30, residues 21-127 of SEQ ID NO:44, residues 143-264 of SEQ ID NO:44, residues 1-121 of SEQ ID NO:354 and residues 134-239 of SEQ ID NO:354. In order, the above sequences are the amino acid sequences of anti-CD20 antibody V_{L} (2H7), anti-CD20 antibody V_{H} (2H7), anti-CD28 antibody V_{H} (2E12), anti-CD28 antibody V_{L} (2E12), anti-CD3 antibody V_{H} (G19-4), anti-CD3 antibody V_{L} (G19-4), anti-CD37 antibody V_{H} (G28-1), anti-CD37 antibody V_{L} (G28-1), anti-CD20 antibody V_{H} (2Lm 20-4) and anti-CD20 antibody V_{L} (2Lm 20-4). The invention comprehends PIMS having a single specific binding domain, by analogy to camelid antibody organization, as well as the more conventional pairing of heavy and light chains for those specific binding domains derived from antibodies. In the latter organization, part or all of a V_{L} domain and part or all of a V_{H} domain are contemplated, provided that the PIMS molecule retains the capacity to specifically bind to a binding partner. Moreover, the V_{L} and V_{H} may be arranged in either orientation and may be separated by at least about 5-8 amino acids using a linker peptide as disclosed herein or any other amino acid sequence capable of providing a spacer function compatible with interaction of the two domains of a PIMS having two such domains, or a multiple thereof. Multi-specific PIMS will have at least two specific binding domains, by analogy to camelid antibody organization, or at least four specific binding domains, by analogy to the more conventional mammalian antibody organization of paired V_{H} and V_{L} chains. In addition, the PIMS proteins may be a specific binding protein, as described above, further comprising a hinge disposed N-terminal to the constant sub-region. In some embodiments, the hinge comprises the same sequence as the PIMS linker disposed between the constant sub-region and the specific binding domain. In some embodiments, the PIMS specific binding protein further comprises at least a second specific binding domain disposed C-terminal to the constant sub-region, and the plurality of specific binding domains may bind to the same or different targets.

Another aspect of the invention is drawn to a method of producing the specific binding protein described herein comprising contacting a cell comprising a polynucleotide encoding the specific binding protein and a culture medium; and incubating the cell in the culture medium under conditions suitable for expression of the polynucleotide.

Yet another aspect of the invention is a method of treating a condition selected from the group consisting of cancer, inflammation and an autoimmune disorder comprising administering an effective amount of a specific binding protein described herein to an organism in need. A preferred organism for treatment is a human. A related aspect of the invention is a use of a specific binding protein as described above in the preparation of a medicament for the treatment of a condition selected from the group consisting of cancer, inflammation an autoimmune disorder. It is contemplated that the medicament may be suitable for administration to a vertebrate such as a mammal, e.g., a human.

Another aspect of the invention is a method of ameliorating a symptom of a condition selected from the group consisting of cancer, inflammation and an autoimmune disorder comprising administering an effective amount of a specific binding protein as described herein to an organism in need. Again, a preferred organism is a human. A related aspect of the invention is a use of a specific binding protein as described above in the preparation of a medicament for ameliorating a symptom of a condition selected from the group consisting of cancer, inflammation an autoimmune disorder. It is contemplated that the medicament may be suitable for administration to a vertebrate such as a mammal, e.g., a human.

Still another aspect of the invention is a use of a specific binding protein as described herein in the preparation of a medicament for the treatment of a disorder selected from the group consisting of cancer, inflammation and an autoimmune disorder. Other features and advantages of the present invention will be better understood by reference to the following detailed description, including the examples.

### BRIEF DESCRIPTION OF THE DRAWING

FIG. 1 presents a schematic illustration of the structure of a specific binding protein with effector function, or PIMS peptide.

FIG. 2 shows a graph of comparative PIMS (W0001), SMIP (2Lm20-4 binding CD20) and Scorpion (SO129, a CD20×CD20 multi-specific binding protein) binding in a CD16 (high affinity) binding ELISA assay revealing mean fluorescent intensity of bound protein as a function of protein concentration.

FIG. 3 is a graph of comparative PIMS (W0001), SMIP (2Lm20-4) and Scorpion (SO129) binding in a CD16 (low affinity) binding ELISA assay revealing mean fluorescent intensity of bound protein as a function of protein concentration.

FIG. 4 shows a graph of comparative binding of PIMS (W001) and SMIP (2E12) to human peripheral CD3+ T-cells.

FIG. 5 shows the mean fluorescence intensity of FITC F′2 GAH (goat anti-human secondary antibody) staining of PE (phycoerythrin)-labeled CD3+lymphocytes by peripheral blood mononuclear cells incubated with the 2E12 SMIP, the 2E12 PIMS, or PE CD3+ and GAH alone. Mean fluorescence intensity was graphed as a function of the concentration of reagent sample (2E12 SMIP, 2E12 PIMS or controls (combination of PE-conjugated anti-CD3 antibody (BD Pharmingen) and goat anti-human secondary antibody) being assayed.

FIG. 6 shows the binding of PIMS to WIL2-S cells. Binding was measured as geometric or geo mean fluorescence intensity as a function of the concentration of PIMS (ug/ml). Solid square: TRU-015 (an anti-CD20 SMIP), open square: 2Lm20-4-scc, solid upright triangle: 2Lm20-4HL17, 2Lm20-4HL12, open upright triangle: PIMS20-17, and open diamond: PIMS20-12.

FIG. 7 shows the effect of PIMS linkers on anti-DR PIMS binding to Wil2-S cells. Binding was measured as geo mean fluorescence intensity as a function of protein concentration (ug/ml) exposed to 500,000 cells. Solid diamond: M0019 (a DR SMIP), solid square: W0035 PIMS, solid upright triangle: W0036 PIMS, and “X”: W0056 PIMS.

FIG. 8 shows the percent of maximal binding to Ramos cells (% maximum binding) as a function of the concentration (nM) of anti-CD37 PIMS molecules contacting the cells. Solid square: TRU-016, an anti-CD37 SMIP, solid upright triangle: W0012 (25-amino-acid PIMS linker based on H7), solid inverted triangle: W0023 (10-amino-acid PIMS linker based on H7), solid diamond: W0024 (15-amino-acid PIMS linker based on H7), solid circle: W0025 (20-amino-acid PIMS linker based on H7), open square: W0094 (25-amino-acid PIMS linker based on H65), open upright triangle: W0095 (10-amino-acid PIMS linker based on H65), open inverted triangle: W0096 (15-amino-acid PIMS linker based on H65), and open circle: W0097 (20-amino-acid PIMS linker based on H65).

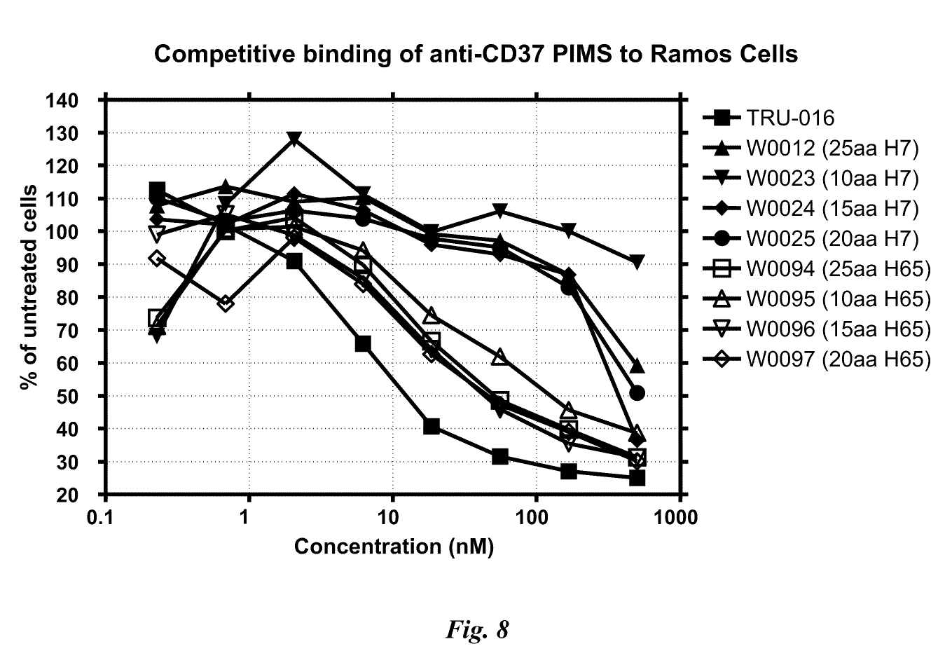

FIG. 9 shows the binding of various PIMS molecules and SMIPs to Ramos B-cells, as revealed by the mean fluorescence intensity following fluorescence labeling using an enzyme-conjugated secondary antibody. Open square: aHer2 (anti-Her2), solid square: TRU-016 (an anti-CD37 SMIP), W0028 (a mouse anti-CD37 PIMS), open diamond: W0029 (a hemi-humanized anti-CD19 PIMS), solid diamond: W0030-HD37 (a mouse anti-CD19), and solid circle: W0031-4G7 (a mouse anti-CD19).

FIG. 10 shows the binding of anti-CD28 PIMS to Jurkat T-cells. Binding was measured by the mean fluorescence intensity and the MFI was plotted as a function of the concentration of protein (ug/ml). Solid diamond: W0001 (H7 PIMS linker), solid square: W0050 (H9 PIMS linker), solid upright triangle: W0051 (H47 PIMS linker), square with corner projections: W0052, square with corner projections and projection top-center: W0053 (H62 PIMS linker), solid circle: W0083 (H65 PIMS linker), and solid square with vertical tick mark through square center: anti-CD28 SMIP.

FIG. 11 shows the binding of CD16Lo to CAS PIMS bound to Ramos cells. The Geo mean fluorescence intensity as a function of the concentration of PIMS is shown. Diamond (1): TRU-016 (an anti-CD37 SMIP), square (2): W0012, upright triangle (3): W0023, X-mark (4): W0024, asterisk (5): W0025, diamond (6): W0094, vertical tick mark (7): W0095, small rectangle (8): W0096, and large rectangle (9): W0097.

FIG. 12 shows anti-CD20 PIMS-mediated ADCC of BJAB B-cells. The percent specific BJAB B-cell killing as a function of the concentration of PIMS is plotted. Blue diamond: W0008 (10-amino-acid PIMS linker), red circle: W0009 (15-amino-acid PIMS linker), green upright triangle: 2Lm20-4 SMIP, black circle: medium.

FIG. 13 shows anti-CD28 PIMS-mediated ADCC of Jurkat T-cells. The percent specific Jurkat T-cell killing as a function of the concentration of PIMS is shown. Diamond: W0001 (anti-CD28 PIMS), square: 2E121g (an anti-CD28 SMIP), triangle: 2E12 N297D Ig (a variant anti-CD28 SMIP), and circle: media.

FIG. 14 shows anti-DR PIMS-mediated ADCC of BJAB B-cells. The percent specific BJAB B-cell killing as aufunction of the concentration of PIMS is provided. Diamond: M0019 (F3.3 SMIP), square: W0035 (F3.3H7 PIMS), triangle: W0036 (F3.3 NKG2A PIMS), W0056 (F3.3 NKG2A cys K/O PIMS), square with dased line: rituximab, and square without connecting line: media.

FIG. 15 shows the anti-CD37 PIMS-mediated ADCC of BJAB B-cells. The percent specific cell killing was measured as a function of protein concentration (nM). Diamond: W0012 (H7 PIMS linker), square: W0094 (H65 PIMS linker), upright triangle: TRU-016 (an anti-CD37 SMIP), circle with dashed line: rituximab, and circle with solid line: media.

FIG. 16 shows PIMS-mediated CDC of Ramos B-cells in the presence of human serum. The percent propidium iodide (PI) positive cells as a function of the concentration (ug/ml) of PIMS was plotted. Diamond: 2Lm20-4 SMIP (humanized anti-CD20 SMIP), square: W0008 (10-amino-acid PIMS linker; HL binding domain orientation), upright triangle: W0009 (15-amino-acid PIMS linker; HL binding domain orientation), solid circle: TRU-015 (an anti-CD20 SMIP), and open circle: media.

FIG. 17 shows that anti-DR PIMS inhibit ATP release in DHL-6 B-cells. Relative Luminescence Unit (RLU) was measured as a function of protein concentration (ug/ml). Solid diamond: F3.3 SMIP+GAH (goat anti-human secondary antibody), open square: F3.3H7 PIMS+GAH, open upright triangle: F3.3 NKG2A PIM+GAH, solid circle: TDR31.1 monoclonal antibody+GAM (goat anti-mouse secondary antibody), solid upright triangle: GAM (3:1), solid square:GAH (3:1), and open circle: medium.

FIG. 18 shows the binding of anti-Her2 PIMS to SKBR3 cells. Binding was measured as mean fluorescence intensity as a function of the concentration of protein (ug/ml). Solid diamond: W0042 PIMS, solid square: W0044 PIMS, solid upright triangle: W0045 PIMS, solid large square: W0041 PIMS, and solid large square: Her033 SMIP.

FIG. 19 shows the binding of anti-Her2 PIMS to MDA-MB453 cells. Binding was measured as mean fluorescence intensity as a function of the concentration of protein (ug/ml). Solid diamond: HerO33 SMIP, solid small square: W0042, solid upright triangle: W0057, solid large square: control.

### DETAILED DESCRIPTION OF THE INVENTION

The invention provides molecules that combine a capacity to specifically bind a given target, such as a target on an undesirable cell (e.g., a cancer cell or a cell involved in inflammation or autoimmune disorders), with an antibody effector-like activity in a manner that places a constant sub-region towards the N-terminus of the molecule and the specific binding domain towards the C-terminus of the molecule, with a (typically hinge-like) PIMS linker region joining the specific binding domain and the constant sub-region, the linker region optionally containing at least one cysteine capable of forming at least one disulfide bond. The effector-like functions include ADCC (antibody-dependent cellular cytotoxicity) and CDC (complement-dependent cytotoxicity), thus associating cytotoxicity with deleterious cells (e.g., cancer cells and cells causing or exacerbating inflammation or autoimmune disorders). The molecules are effective in that activity is not lost by placing the constant sub-region towards the N-terminus, with the C-terminus of the constant sub-region in peptide linkage to the remainder of the molecule, unlike any antibody organization found in nature, where the constant region is located at the C-terminus of the molecule. The molecules also are typically smaller than native antibodies and similar polypeptides, thus potentially improving the penetrance of the molecule, herein referred to as a reverse Small Modular ImmunoPharmaceutical (i.e., SMIP) or PIMS molecule. Notably, the small size does not compromise the in vivo persistence of PIMS as is found for other small peptide molecules contemplated for administration to organisms. Without wishing to be bound by any theory, the presence of the constant sub-region may contribute to the in vivo durability of the molecules.

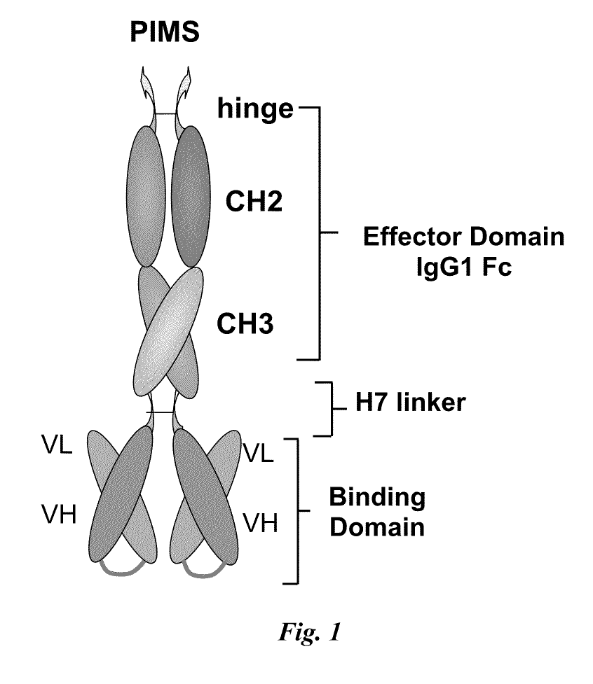

The schematic structure of an exemplary PIMS molecule is indicated in FIG. 1. In general, a PIMS molecule is a single-chain polypeptide comprising, in N-terminal to C-terminal orientation, a constant sub-region derived from an immunoglobulin(s) that includes a C_{H2} and C_{H3} domain from the same (preferred) or different animal species, immunoglobulin isotype and/or immunoglobulin sub-class, a PIMS linker peptide that is typically a hinge region derived from an immunoglobulin, and a specific binding domain from a peptide member of a specific binding pair, which includes one or more regions derivable from the peptide member that collectively contribute to a functional binding domain. In some embodiments, the PIMS molecule further contains an N-terminally disposed hinge region derivable from an immunoglobulin, and the N-terminal hinge region may be the same as, or different than, the PIMS linker region found between the constant sub-region and the binding domain. Still further, a PIMS molecule may contain an N-terminal leader sequence useful in polypeptide production, and that leader sequence may include amino acids encoded by engineered restriction endonuclease cleavage sites found in an encoding nucleic acid. Thus, exemplary schematic organizations of some PIMS molecules include the following: N-constant sub-region-PIMS linker-binding domain-C, N-hinge-constant sub-region-PIMS linker-binding domain-C, N-leader-constant sub-region-PIMS linker-binding domain-C, or N-leader-hinge-constant sub-region-PIMS linker-binding domain-C. The functional components of PIMS molecules will now be described in greater detail.

The invention also provides compositions comprising a polynucleotide encoding a PIMS along with a vector comprising such a polynucleotide and a host cell comprising such a polynucleotide or vector, methods of making such molecules, preferably through recombinant protein production methods, but also through chemical syntheses in view of the relatively small size of the proteins. Further, the invention provides methods of treating disorders such as cancer, inflammation and autoimmune disorders, as well as related methods of ameliorating a symptom of such disorders.

Provided with such molecules, and the methods of recombinantly producing them in vivo, new approaches to targeted diagnostics and therapeutics have become available that allow, e.g., for the targeted recruitment of effector cells of the immune system (e.g., cytotoxic T lymphocytes, natural killer cells, and the like) to cells, tissues, agents and foreign objects to be destroyed or sequestered, such as cancer cells and infectious agents. In addition to localizing therapeutic cells to a site of treatment, the peptides are useful in localizing therapeutic compounds, such as radiolabeled proteins. Further, the peptides are also useful in scavenging deleterious compositions, for example by associating a deleterious composition, such as a toxin, with a cell capable of destroying or eliminating that toxin (e.g., a macrophage). The molecules of the invention are useful in modulating the activity of binding partner molecules, such as cell surface receptors. Diseases and conditions where the elimination of defined cell populations is beneficial would include infectious and parasitic diseases, inflammatory and autoimmune conditions, malignancies, and the like. Further consideration of the disclosure of the invention will be facilitated by a consideration of the following express definitions of terms used herein.

A “single-chain binding protein” is a single contiguous arrangement of covalently linked amino acids, with the chain capable of specifically binding, as a monomer and/or multimer, to one or more binding partners sharing sufficient determinants of a binding site to be detectably bound by the single-chain binding protein. Exemplary binding partners include proteins, carbohydrates, lipids and small molecules.

For ease of exposition, “derivatives” and “variants” of proteins, polypeptides, and peptides according to the invention are described in terms of differences from proteins and/or polypeptides and/or peptides according to the invention, meaning that the derivatives and variants, which are proteins/polypeptides/peptides according to the invention, differ from underivatized or non-variant proteins, polypeptides or peptides of the invention in the manner defined. One of skill in the art would understand that the derivatives and variants themselves are proteins, polypeptides and peptides according to the invention.

An “antibody” is given the broadest definition consistent with its meaning in the art, and includes proteins, polypeptides and peptides capable of binding to at least one binding partner, such as a proteinaceous or non-proteinaceous antigen. An “antibody” as used herein includes members of the immunoglobulin superfamily of proteins, of any species, of single- or multiple-chain composition, and variants, analogs, derivatives and fragments of such molecules. Specifically, an “antibody” includes any form of antibody known in the art, including but not limited to, monoclonal and polyclonal antibodies, chimeric antibodies, CDR-grafted antibodies, humanized antibodies, human antibodies, single-chain variable fragments, bi-specific antibodies, diabodies, antibody fusions, and the like.

A “binding domain” is a peptide region, such as a fragment of a polypeptide derived from an immunoglobulin (e.g., an antibody), that specifically binds one or more specific binding partners. If a plurality of binding partners exists, those partners share binding determinants sufficient to detectably bind to the binding domain. Preferably, the binding domain is a contiguous sequence of amino acids.

An “epitope” is given its ordinary meaning herein of a single antigenic site, i.e., an antigenic determinant, on a substance (e.g., a protein) with which an antibody specifically interacts, for example by binding. Other terms that have acquired well-settled meanings in the immunoglobulin (e.g., antibody) art, such as a “variable light region,” variable heavy region,” “constant light region,” constant heavy region,” “antibody hinge region,” “complementarity determining region,” “framework region,” “antibody isotype,” “Fc region,” “constant region,” “single-chain variable fragment” or “scFv,” “diabody,” “chimera,” “CDR-grafted antibody,” “humanized antibody,” “shaped antibody,” “antibody fusion,” and the like, are each given those well-settled meanings known in the art, unless otherwise expressly noted herein.

Terms understood by those in the art as referring to antibody technology are each given the meaning acquired in the art, unless expressly defined herein. Examples of such terms are “V_{L}” and “V_{H}”, referring to the variable binding region derived from an antibody light and heavy chain, respectively; and C_{L} and C_{H}, referring to an “immunoglobulin constant region,” i.e., a constant region derived from an antibody light or heavy chain, respectively, with the latter region understood to be further divisible into C_{H1}, C_{H2}, C_{H3} and C_{H4} constant region domains, depending on the antibody isotype (IgA, IgD, IgE, IgG, IgM) from which the region was derived. CDR means “complementarity determining region.” A “hinge region” is derived from the amino acid sequence interposed between, and connecting, the C_{H1} and C_{H2} regions of a single chain of an antibody, which is known in the art as providing flexibility, in the form of a “hinge,” to whole antibodies.

A “constant sub-region” is a term defined herein to refer to a peptide, polypeptide, or protein sequence that corresponds to, or is derived from, part or all of one or more constant region domains, but not all constant region domains, of the source/parent antibody to which the constant sub-region polypeptide corresponds. Thus, a constant sub-region may include part or all of any of the following domains: a C_{H2} domain, a C_{H3} domain (IgA, IgD, IgG, IgE, or IgM), and a C_{H4} domain (IgE or IgM). A constant sub-region as defined herein, therefore, can refer to a polypeptide that corresponds to a portion of an immunoglobulin constant region, provided it retains at least one effector function associated with an antibody. Typically, a constant sub-region of a polypeptide, or encoding nucleic acid, of the invention has a C_{H2} domain and C_{H3} domain, although a PIMS molecule may optionally have an N-terminal hinge region linked to the constant sub-region. In some embodiments, the constant sub-region is disposed N-terminal to the one or more specific binding domains of the molecule. Although there may be some sequence N-terminal to the constant sub-region, such as the N-terminal hinge noted above, in these embodiments there is no specific binding domain N-terminal to the constant sub-region.

An “effector function” is a function associated with or provided by a constant region of an antibody. Exemplary effector functions include antibody-dependent cell-mediated cytotoxicity (ADCC), complement activation and complement-dependent cytotoxicity (CDC), F_{C} receptor binding, and increased plasma half-life, as well as placental transfer. An effector function of a composition according to the invention is provided by the constant sub-region and is detectable; preferably, the specific activity of the composition according to the invention for that function is about the same as the specific activity of a wild-type antibody with respect to that effector function, i.e., the constant sub-region of the PIMS molecule preferably has not lost any effector function relative to a wild-type antibody.

A “linker” is a peptide, or polynucleotide, that joins or links other peptides or polynucleotides. Typically, a peptide linker is an oligopeptide of from about 2-50 amino acids, with typical polynucleotide linkers encoding such a peptide linker and, thus, being about 6-150 nucleotides in length. A PIMS linker is a type of peptide linker that joins an immunoglobulin-derived constant subregion to a binding domain, or to the binding domain located closest to the N-terminus of the specific binding peptide when more than one binding domain is present. This PIMS linker has an amino acid sequence derived from an antibody hinge region, or derived from a region linking a binding domain, or derived from a region linking a binding domain to a cell surface transmembrane region or membrane anchor. In some embodiments, the PIMS linker has at least one cysteine residue capable of participating in at least one disulfide bond under standard peptide conditions (e.g., physiological conditions, conventional peptide purification conditions, conventional conditions for peptide maintenance or storage, and the like). Preferably, the disulfide bond(s) formed by these cysteine(s) are inter-chain disulfide bonds. Binding domain linkers, which may be the same or different from the above-described linker interposed between the constant subregion and the N-terminal specific binding domain, may themselves be interposed between all binding domains in a protein containing more than one binding domain. Exemplary binding domain linkers are peptides belonging to the (Gly4Ser)n family where, preferably, n=1-5.

A “target” is given more than one meaning, with the context of usage defining an unambiguous meaning in each instance. In its narrowest sense, a “target” is a binding site, i.e., the binding domain of a binding partner for a peptide composition according to the invention. In a broader sense, “target” or “molecular target” refers to the entire binding partner (e.g., a protein), which necessarily exhibits the binding site. Specific targets, such as “CD20,” “CD37,” and the like, are each given the ordinary meaning the term has acquired in the art. A “target cell” is any prokaryotic or eukaryotic cell, whether healthy or diseased, that is associated with a target molecule according to the invention. Of course, target molecules are also found unassociated with any cell (i.e., a cell-free target) or in association with other compositions such as viruses (including bacteriophage), organic or inorganic target molecule carriers, and foreign objects.

Examples of materials with which a target molecule may be associated include autologous cells (e.g., cancer cells or other diseased cells), infectious agents (e.g., infectious cells and infectious viruses), and the like. A target molecule may be associated with an enucleated cell, a cell membrane, a liposome, a sponge, a gel, a capsule, a tablet, and the like, which may be used to deliver, transport or localize a target molecule, regardless of intended use (e.g., for medical treatment, as a result of benign or unintentional provision, or to further a bioterrorist threat). “Cell-free,” “virus-free,” “carrier-free,” “object-free,” and the like refer to target molecules that are not associated with the specified composition or material.

“Binding affinity” refers to the strength of non-covalent binding of the peptide compositions of the invention and their binding partners. Preferably, binding affinity refers to a quantitative measure of the attraction between members of a binding pair.

An “adjuvant” is a substance that increases or aids the functional effect of a compound with which it is in association, such as in the form of a pharmaceutical composition comprising an active agent and an adjuvant. An “excipient” is an inert substance used as a diluent in formulating a pharmaceutical composition. A “carrier” is a typically inert substance used to provide a vehicle for delivering a pharmaceutical composition.

“Host cell” refers to any cell, prokaryotic or eukaryotic, in which is found a polynucleotide, protein or peptide according to the invention.

“Introducing” a nucleic acid or polynucleotide into a host cell means providing for entry of the nucleic acid or polynucleotide into that cell by any means known in the art, including but not limited to, in vitro salt-mediated precipitations and other forms of transformation or transfection of naked nucleic acid/polynucleotide or vector-borne nucleic acid/polynucleotide, virus-mediated infection and optionally transduction, with or without a “helper” molecule, ballistic projectile delivery, conjugation, and the like.

“Incubating” a host cell means maintaining that cell under environmental conditions known in the art to be suitable for a given purpose, such as gene expression. Such conditions, including temperature, ionic strength, oxygen tension, carbon dioxide concentration, nutrient composition, and the like, are well known in the art.

“Isolating” a compound, such as a protein or peptide according to the invention, means separating that compound from at least one distinct compound with which it is found associated in nature, such as in a host cell expressing the compound to be isolated, e.g. by isolating spent culture medium containing the compound from the host cells grown in that medium.

An “organism in need” is any organism at risk of, or suffering from, any disease, disorder or condition that is amenable to treatment or amelioration with a composition according to the invention, including but not limited to any of various forms of cancer, any of a number of autoimmune diseases, radiation poisoning due to radiolabeled proteins, peptides and like compounds, ingested or internally produced toxins, and the like, as will become apparent upon review of the entire disclosure. Preferably, an organism in need is a human patient.

“Ameliorating” a symptom of a disease means detectably reducing the severity of that symptom of disease, as would be known in the art. Exemplary symptoms include pain, heat, swelling and joint stiffness.

Unless clear from context, the terms “protein,” “peptide,” and “polypeptide” are used interchangeably herein, with each referring to at least one contiguous chain of amino acids. Analogously, the terms “polynucleotide,” “nucleic acid,” and “nucleic acid molecule” are used interchangeably unless it is clear from context that a particular, and non-interchangeable, meaning is intended.

“Pharmaceutically acceptable salt” refers to salts of the compounds of the present invention derived from the combination of such compounds and an organic or inorganic acid (acid addition salts) or an organic or inorganic base (base addition salts).

In some embodiments, polynucleotides useful in the recombinant expression of a PIMS molecule will encode a leader peptide linked to an N-terminal hinge region (e.g., the IgG1 hinge) by sequences encoding varying short di- or tri-peptides that include the sequences of restriction endonuclease cleavage sites useful in recombinant DNA engineering of the molecules, such as an AgeI site (e.g., at the end of a leader-encoding sequence) and an XhoI site (e.g., at the beginning of a sequence encoding a hinge such as an IgG1 hinge) followed by the hinge (e.g., SCC-P), C_{H2} and C_{H3} domains (e.g., IgG1 domains) fused to an scFv by a (hinge-like) PIMS linker such as the H7 Scorpion linker (having an amino acid sequence of SEQ ID NO:164 encoded, e.g., by SEQ ID NO:163). In this way, a PIMS molecule can be seen to resemble a “backwards” SMIP at a crude structural level, although a PIMS molecule typically contains two hinge-like disulfide bonding regions, one N-terminal and the other lying between the end of the effector domain and the specific binding domain or domains.

### PIMS Binding Domain

A PIMS protein or polypeptide has a structure unlike the structure of other specific binding molecules, such as polyclonal or monoclonal antibodies, fragments thereof such as Fab, F(ab′)₂, or scFv, SMIPs, diabodies, scorpions, and the like. A PIMS protein or polypeptide contains an effector domain (C_{H2}-C_{H3}, optionally hinge—C_{H2}-C_{H3}), at the N-terminus and a target-specific (e.g., antigen-specific) binding domain at the C-terminus with both domains separated by a (hinge-like) PIMS linker. Where the PIMS linker is derived from an Ig hinge, it is preferably derived from the same antibody class, isotype, or sub-isotype as at least one of the C_{H2} and C_{H3} domains of that PIMS molecule. The target-specific binding domain may be a single domain, e.g., a binding domain derived from a camelid antibody binding domain, or more typically, a plurality of regions may associate to form at least one binding domain, such as the inter- or intra-chain association of a V_{L}-like domain and a V_{H}-like domain. A PIMS structure allows optimization of both effector function and the functional characteristics of the binding domain. These molecules have activity on their own as well as offering a platform for assessing a binding domain of a multi-specific binding protein, such as the C-terminal binding domain of a Scorpion (i.e., a Scorpion binding domain 2 (BD2)), as well as for assessing the effector domain-BD2 Scorpion linker.

The specific binding domain may be derived from one or more regions of a protein or polypeptide member of a specific binding pair. Typically, a binding domain is derived from at least one region of the same, or different, immunoglobulin protein structures such as antibody molecules. The specific binding domain may exhibit a sequence identical to the sequence of a region of an immunoglobulin, or may be a modification of such a sequence to provide, e.g., altered binding properties or altered stability. Such modifications are known in the art and include alterations in amino acid sequence that contribute directly to the altered property such as altered binding, for example, by leading to an altered secondary or higher order structure for the peptide. Also contemplated are modified amino acid sequences resulting from the incorporation of non-native amino acids, such as non-native conventional amino acids, unconventional amino acids and imino acids. In some embodiments, the altered sequence results in altered post-translational processing, leading, for example, to an altered glycosylation pattern.

For embodiments in which a binding domain is derived from more than one region of an immunoglobulin (e.g., an Ig V_{L} region and an Ig V_{H} region), the plurality of regions may be joined by a linker peptide, which is described below. The structures of exemplary, but non-limiting, binding domains suitable for use in the compositions of the invention are provided in Table 1.

TABLE 1

| | | Sequence Identifier |
|---|---|---|
| | Exemplary molecule comprising | (exemplary encoding |
| Binding domain | domain | nucleic acid) |
| Anti-CD20 variable | 2H7 (H₃N-VL-VH-CO₂H orientation) | 120 (119) |
| region | | |
| Anti-CD20 (2Lm20-4) | 2Lm20-4 (H₃N-VH-VL-CO₂H orientation) | 122 (121) |
| HL 12 | | |
| Anti-CD20 (2Lm20-4) | 2Lm20-4 (H₃N-VH-VL-CO₂H orientation) | 124 (123) |
| HL 17 | | |
| Anti-CD28 variable | 2E12 (H₃N-VL-VH-CO₂H orientation) | 126 (125) |
| region | | |
| Anti-CD28 variable | 2E12 (H₃N-VH-VL-CO₂H orientation) | 128 (127) |
| region | | |
| Anti-CD37 variable | G28-1 (H₃N-VL-VH-CO₂H orientation) | 130 (129) |
| domain | | |
| Anti-CD37 variable | G28-1 (H₃N-VH-VL-CO₂H orientation) | 132 (131) |
| domain | | |
| Anti-CD3 variable | G19-4 (H₃N-VL-VH-CO₂H orientation) | 134 (133) |
| domain | | |
| Anti-CD3 variable | G19-4 (H₃N-VH-VL-CO₂H orientation) | 136 (135) |
| domain | | |

### PIMS Constant Sub-Region

The constant sub-region, disposed towards or at the N-terminus of the polypeptide, is derived from a constant region of an immunoglobulin protein. The constant sub-region generally is derived from part, but not all, of a constant heavy chain region of an immunoglobulin. Typically, the constant sub-region contains a hinge-CH₂ portion of a C_{H} region of an immunoglobulin, although it may be derived from a hinge-C_{H2}-C_{H3} portion or it may be derived from a hinge-partial C_{H2} portion of a C_{H} region, provided the constant sub-region retains at least one effector function associated with an antibody. Also, portions of the constant sub-region may be derived from the C_{H} regions of different immunoglobulins. In preferred embodiments, a hinge and C_{H2} region, as well as a C_{H3} region where relevant, are derived from the same antibody isotype. Further contemplated are molecules having an N-terminal leader sequence joined to a constant subregion or an N-terminal hinge. The constant sub-region provides at least one activity associated with a C_{H} region of an immunoglobulin, such as one or more of the following effector functions: antibody-dependent cell-mediated cytotoxicity (ADCC), complement-dependent cytotoxicity (CDC), protein A binding, binding to at least one Fc receptor, reproducibly detectable stability relative to a protein according to the invention except for the absence of a constant sub-region, and perhaps placental transfer where generational transfer of a molecule according to the invention would be advantageous, as recognized by one of skill in the art. Constant sub-regions suitable for use in the compositions of the invention include, but are not limited to, the structures exemplified in Table 2. The constant sub-region is derived from at least one immunoglobulin molecule and exhibits an identical or substantially identical amino acid sequence to a region or regions of at least one immunoglobulin. In some embodiments, the constant sub-region is modified from the sequence or sequences of at least one immunoglobulin (by substitution of one or more non-native conventional or unconventional, e.g., synthetic, amino acids or imino acids), resulting in a primary structure that may yield an altered secondary or higher order structure with altered properties associated therewith, or may lead to alterations in post-translational processing, such as glycosylation.

Exemplary modified constant sub-regions are illustrated in Table 2 and include hIgG1 (P238S)-C_{H2}C_{H3} (SEQ ID NO:142, encoded e.g., by SEQ ID NO:141), hIgG1 (P331S)-C_{H2}C_{H3} (SEQ ID NO:144 encoded, e.g., by SEQ ID NO:143) and hIgG1 (P238S/P331S)-C_{H2}C_{H3} (SEQ ID NO:146 encoded, e.g., by SEQ ID NO:145). Note that “P238S” refers to the substitution of a Pro residue by a Ser residue at position 238 using the Kabat numbering system. The 238 position (Kabat) is at position 8 of SEQ ID NOS:142 and 146, and codons encoding the position 8 (Kabat 238) residue are found at the corresponding positions in exemplary encoding nucleic acids (SEQ ID NOS:141 and 145). Analogously, the P331S substitution is at position 331 (Kabat), which is at position 101 of SEQ ID NO:144 and 146, with cognate codons at corresponding locations in exemplary encoding nucleic acids (SEQ ID NOS:143 and 145).

For those binding domains and constant sub-regions exhibiting an identical or substantially identical amino acid sequence to one or more immunoglobulin polypeptides, the post-translational modifications of the molecule according to the invention may result in a molecule modified relative to the immunoglobulin(s) serving as a basis for modification. For example, using techniques known in the art, a host cell may be modified, e.g., a CHO cell, in a manner that leads to an altered polypeptide glycosylation pattern relative to that polypeptide in an unmodified (e.g., CHO) host cell.

TABLE 2

| | Sequence Identifier (exemplary |
|---|---|
| Effector domain | encoding nucleic acid |
| hIgG1-C_{H2}C_{H3} | 140 (139) |
| hIgG1 (P238S)-C_{H2}C_{H3} | 142 (141) |
| hIgG1 (P331S)-C_{H2}C_{H3} | 144 (143) |
| hIgG1 (P238S/P331S)-C_{H2}C_{H3} | 146 (145) |

### PIMS Hinge Region

The actual structures of PIMS linker regions of typical PIMS molecules are provided in Table 3, wherein the amino acid sequence and, where applicable, an encoding polynucleotide sequence are provided for a non-limiting number of suitable regions. Typically, these PIMS linker regions are derived from the hinge region of an immunoglobulin. Also typically, the sequence of the PIMS linker includes a cysteine residue capable of forming a disulfide bond, and preferably an inter-chain disulfide bond, under standard peptide conditions. PIMS molecules comprising a hinge-like PIMS linker having at least one cysteine promote homodimerization and effector function. Unless otherwise indicated, sequences in Table 3 are from IgG1 hinge regions. There is potential for affecting ADCC and CDC activities through PIMS modifications, which is expected to include changes to either an N-terminal hinge (e.g., an IgG1 SCC-P hinge [SEQ ID NO:81]) or to a hinge-like PIMS linker separating the constant sub-region from the specific binding domain (e.g., a Scorpion linker (H7), SEQ ID NO:164, encoded by, e.g., SEQ ID NO:163). Variants have been constructed to optimize the region surrounding the signal sequence (leader) to direct post-translation cleavage to the N-terminus of the mature PIMS peptide during expression in COS-7 cells. Other variants are envisioned that would alter a sequence corresponding to the IgG1 upper hinge region in a manner that affected expression, aggregation and/or ADCC and/or CDC function. Further, alterations of the PIMS linker region interposed between the effector domain and specific binding domains are also contemplated to affect expression, aggregation and/or ADCC and/or CDC function, as well as to effect alterations in binding affinity to FcRs.

The PIMS linkers of Table 3 (i.e., hinges 1-26) are variants of the wild-type hinge also provided in that Table. Apparent from an inspection of the various sequences is that variants were created to lengthen or shorten the hinge, to alter the sequence to increase or decrease the probability of interchain bonding by adding or deleting Cys residues, and to alter the flexibility of the hinge by introducing flexibility-promoting residues (e.g., Gly) or flexibility-inhibiting residues (e.g., Pro). Comparing the wild-type IgG1 hinge of Table 3 with hinges 1-26 (see Dall'Acqua et al., J. Immunol. 177:1129-1138 (2006), incorporated herein by reference) revealed that hinges 11, 13, 14, and 17 exhibited lower binding affinity to FcγRIIIA than the wild-type hinge, while hinges 1-10, 12, 15-16, and 18-26 exhibited a binding affinity to FcγRIIIA that was comparable to the wild-type hinge. Accordingly, one of skill in the art would compare the sequences in Table 3 to design a hinge-like PIMS linker based on IgG1 hinge sequences known to exhibit an approximately wild-type binding affinity for FcγRIIIA or known to exhibit a lowered binding affinity for FcγRIIIA. That binding affinity, moreover, directly correlates with ADCC activity and, hence, one of skill would be able to design a PIMS linker region associated with an approximately wild-type level of ADCC activity, or a PIMS linker associated with less ADCC activity. In comparing sequences to finalize the design of a PIMS linker, or hinge, any one of a number of standard software programs and packages suitable for the purpose could be chosen to facilitate arrival at a suitable design, and such sequences are contemplated as components of the compositions of the invention.

Hinge-like PIMS linker regions having one of the sequences of Table 3 also exhibit varying levels of binding affinity for C1_{q} and, hence, varying level of complement-mediated cytotoxicity. Hinges 19, 21-22, and 24-25 exhibit a higher binding affinity for C1_{q} than the wild-type hinge; hinges 3-6, 11, 13-14, and 18 exhibit a lower binding affinity for Clq than the wild-type hinge; and hinges 1-2, 7-10, 12, 15-17, 20, 23, and 26 exhibit a binding affinity for C1_{q} that is comparable to the binding affinity of the wild-type hinge for that complement protein. Accordingly, relative to the CDC activity associated with the wild-type IgG1 hinge, hinges 19, 21-22, and 24-25 are associated with a higher CDC activity, hinges 3-6, 11, 13-14, and 18 are associated with a lower CDC activity, and hinges 1-2, 7-10, 12, 15-17, 20, 23, and 26 are associated with about the same level of CDC activity as the wild-type hinge. As in designing PIMS linkers or hinges for contributions to relative binding to FcγRIIIA and/or ADCC levels, one of skill in the art would know to compare the PIMS linker or hinge sequences disclosed herein and, based on the differing binding affinities for C1_{q} and/or differing levels of CDC, the design could be optimized to achieve a relative level of affinity for C1_{q} binding and/or CDC using well-known algorithms implemented in readily available software programs and packages. For some of the hinges described above, Dall'Acqua et al. confirmed the binding observations by determining dissociation constants. Hinges exhibiting lowered binding affinity for either FcγRIIIA or C1_{q} also exhibited higher K_{D}S for those molecules.

Changes are also contemplated in the specific binding domain(s) region, such as in an scFv linker region between the V_{H} and V_{L} domains, and these changes may be introduced into a PIMS molecule separately or in combination with the above variants in order to affect binding affinity, expression levels, aggregation tendencies or overall functionality of the molecule.

TABLE 3

| | | | SEQ ID NOS. |
|---|---|---|---|
| | | | (exemplary |
| | | | encoding |
| Hinge | | | nucleic |
| Region | Nucleotide Sequence | Amino Acid Sequence | acid) |
| ccc- | | | 63 |
| hIgG1 | | | |
| sss (s)- | gagcccaaatcttctgacaaaact | EPKSSDKTHTSPPSS | 89 (88) |
| hIgG1 | cacacatctccaccgagctca | | |
| csc (s)- | gagcccaaatcttgtgacaaaact | EPKSCDKTHTSPPCS | 91 (90) |
| hIgG1 | cacacatctccaccgtgctca | | |
| ssc (s)- | gagcccaaatcttctgacaaaact | EPKSSDKTHTSPPCS | 65 (64) |
| hIgG1 | cacacatctccaccgtgctca | | |
| scc (s)- | gagcccaaatcttctgacaaaact | EPKSSDKTHTCPPCS | 67 (66) |
| hIgG1 | cacacatgtccaccgtgctca | | |
| css (s)- | gagcccaaatcttgtgacaaaact | EPKSCDKTHTSPPSS | 69 (68) |
| hIgG1 | cacacatctccaccgagctca | | |
| scs (s)- | gagcccaaatcttgtgacaaaact | EPKSSDKTHTCPPSS | 71 (70) |
| hIgG1 | cacacatgtccaccgagctca | | |
| ccc (s)- | gagcccaaatcttgtgacaaaact | EPKSCDKTHTSPPCS | 73 (72) |
| hIgG1 | cacacatgtccaccgtgctca | | |
| ccc (p)- | gagcccaaatcttgtgacaaaact | EPKSCDKTHTSPPCP | 75 (74) |
| hIgG1 | cacacatgtccaccgtgccca | | |
| sss (p)- | gagcccaaatcttctgacaaaact | EPKSSDKTHTSPPSP | 77 (76) |
| hIgG1 | cacacatctccaccgagccca | | |
| csc (p)- | gagcccaaatcttgtgacaaaact | EPKSCDKTHTSPPCP | 62 (61) |
| hIgG1 | cacacatctccaccgtgccca | | |
| ssc (p)- | gagcccaaatcttctgacaaaact | EPKSSDKTHTSPPCP | 79 (78) |
| hIgG1 | cacacatctccaccgtgccca | | |
| scc (p)- | gagcccaaatcttctgacaaaact | EPKSSDKTHTCPPCP | 81 (80) |
| hIgG1 | cacacatgtccaccgtgccca | | |
| css (p)- | gagcccaaatcttgtgacaaaact | EPKSCDKTHTSPPSP | 83 (82) |
| hIgG1 | cacacatctccaccgagccca | | |
| scs (p)- | gagcccaaatcttgtgacaaaact | EPKSSDKTHTCPPSP | 85 (84) |
| hIgG1 | cacacatgtccaccgagccca | | |
| scppcp | agttgtccaccgtgccca | SCPPCP | 87 (86) |
| wild-type | | EPKSCDKTHTCPPCP | 92 |
| hinge | | | |
| hinge 1 | | GGGGCDKTHTCPPCP | 93 |
| hinge 2 | | EPKSCGGGGGCPPCP | 94 |
| hinge 3 | | EPKSCDKTHTCGGCP | 95 |
| hinge 4 | | EPKSCDKTHTCPPCG | 96 |
| hinge 5 | | EPKSCDKTHTCPPSP | 97 |
| hinge 6 | | EPKSCDKTHTSPPCP | 98 |
| hinge 7 | | EPKSCDKCHTCPPCP | 99 |
| hinge 8 | | EPKSCDKTCCCPPCP | 100 |
| hinge 9 | | EPKSCPPPPPCPPCP | 101 |
| hinge 10 | | PPPPCDKTHTCPPCP | 102 |
| hinge 11 | | EPKSCDKTHTC--CP | 103 |
| hinge 12 | | EPKSCDK--TCPPCP | 104 |
| hinge 13 | | EPKSCDK--TC--CP | 105 |
| hinge 14 | | EPKSCDKTHTCPGGGPCP | 106 |
| hinge 15 | | EPKSCDGGGKTHTCPPCP | 107 |
| hinge 16 | | EPKSCDPPPKTHTCPPCP | 108 |
| hinge 17 | | EPKSCDKTHTCPPPPPCP | 109 |
| hinge 18 | | EPKSCDKTHTCWWCP | 110 |
| hinge 19 | | EPKSCDWWHTCPPCP | 111 |
| hinge 20 | | EPKCSDKTHTCPPCP | 112 |
| hinge 21 | | EPKSDCKTHTCPPCP | 113 |
| hinge 22 | | EPKSDCWWHTCPPCP | 114 |
| hinge 23 | | EPKSCDFFHTCPPCP | 115 |
| hinge 24 | | EPKSCDWWWTCPPCP | 116 |
| hinge 25 | | EPKSCWWTHTCPPCP | 117 |
| hinge 26 | | EPWWCDKTHTCPPCP | 118 |

### PIMS Linker Peptide

PIMS linker peptides are frequently hinge regions and, therefore, any hinge region structure defined herein, including those hinge region structures exemplified in Table 3, are suitable for use as PIMS linker peptides. The PIMS linker peptides useful in the compositions of the invention are preferably between about 2-45 amino acids, or 2-38 amino acids, or 5-45 amino acids. For example, the H1 linker is 2 amino acids in length and the STD2 linker is 38 amino acids in length. Moreover, a PIMS linker may be used to join the specific binding domain to the constant sub-region and this PIMS linker may be derived from a hinge region of an immunoglobulin. Given the generally suitable length parameter provided herein, one of skill would be able to alter the sequence and/or length of a given PIMS linker peptide to achieve a desired level of flexibility and/or spacing of the constant sub-region and binding domain, for example to avoid steric hindrance. The structures of exemplary PIMS linker peptides include the hinge regions provided in Table 3 and the peptide linkers provided in Table 4.

TABLE 4

| | | | Sequence |
|---|---|---|---|
| | | | Identifier |
| | | | (exemplary |
| | | | encoding |
| Linker | Nucleotide Sequence | Amino Acid Sequence | nucleic acid) |
| STD1 | aattatggtggcggtggctcgggc | NYGGGGSGGGGSGGGGSGNS | 148 (147) |
| | ggtggtggatctggaggaggtggg | | |
| | agtgggaattct | | |
| STD2 | aattatggtggcggtggctcgggc | NYGGGGSGGGGSGGGGSGNYGGGG | 150 (149) |
| | ggtggtggatctggaggaggtggg | SGGGGSGGGGSGNS | |
| | agtgggaattatggtggcggtggc | | |
| | tcgggcggtggtggatctggagga | | |
| | ggtgggagtgggaattct | | |
| H1 | aattct | NS | 152 (151) |
| H2 | ggtggcggtggctcggggaattct | GGGGSGNS | 154 (153) |
| H3 | aattatggtggcggtggctctggg | NYGGGGSGNS | 156 (155) |
| | aattct | | |
| H4 | ggtggcggtggctcgggcggtggt | GGGGSGGGGSGNS | 158 (157) |
| | ggatctgggaattct | | |
| H5 | aattatggtggcggtggctcgggc | NYGGGGSGGGGSGNS | 160 (159) |
| | ggtggtggatctgggaattct | | |
| H6 | ggtggcggtggctcgggcggtggt | GGGGSGGGGSGGGGSGNS | 162 (161) |
| | ggatctgggggaggaggcagcggg | | |
| | aattct | | |
| H7 | gggtgtccaccttgtccgaattct | GCPPCPNS | 164 (163) |
| H8 | gggtctccaccttctccgaattct | GSPPSPNS | 166 (165) |
| H9 | tctccaccttctccgaattct | SPPSPNS | 168 (167) |
| H11 | gagcccacatctaccgacaaaact | EPTSTDKTHTSPPSPNS | 172 (171) |
| | cacacatctccacccagcccgaat | | |
| | tct | | |
| H12 | gagcccacatctaccgacaaaact | EPTSTDKTHTCPPCPNS | 174 (173) |
| | cacacatctccacccagcccgaat | | |
| | tct | | |
| H13 | gagcccacatctaccgacaaaact | EPKSSDKTHTCPPCPNS | 176 (175) |
| | cacacatctccacccagcccgaat | | |
| | tct | | |
| H15 | ggcggtggtggctcctgtccacct | GGGGSCPPCPNS | 180 (179) |
| | tgtccgaattct | | |
| H16 | ctgtctgtgaaagctgacttcctc | LSVKADFLTPSIGNS | 182 (181) |
| | actccatccatcgggaattct | | |
| H17 | ctgtctgtgaaagctgacttcctc | LSVKADFLTPSISCPPCPNS | 184 (183) |
| | actccatccatctcctgtccacct | | |
| | tgcccgaattct | | |
| H18 | ctgtctgtgctcgctaacttcagt | LSVLANFSQPEIGNS | 186 (185) |
| | cagccagagatcgggaattct | | |
| H19 | ctgtctgtgctcgctaacttcagt | LSVLANFSQPEISCPPCPNS | 188 (187) |
| | cagccagagatctcctgtccacct | | |
| | tgcccgaattct | | |
| H20 | ctgaaaatccaggagagggtcagt | LKIQERVSKPKISNS | 190 (189) |
| | aagccaaagatctcgaattct | | |
| H21 | ctgaaaatccaggagagggtcagt | LKIQERVSKPKISCPPCPNS | 192 (191) |
| | aagccaaagatctcctgtccacct | | |
| | tgcccgaattct | | |
| H22 | ctggatgtgagtgagaggcctttt | LDVSERPFPPHIQNS | 194 (193) |
| | cctccacacatccagaattct | | |
| H23 | ctggatgtgagtgagaggcctttt | LDVSERPFPPHIQSCPPCPNS | 196 (195) |
| | cctccacacatccagtcctgtcca | | |
| | ccttgcccgaattct | | |
| H24 | cgggaacagctggcagaggtcact | REQLAEVTLSLKANS | 198 (197) |
| | ttgagcttgaaagcgaattct | | |
| H25 | cgggaacagctggcagaggtcact | REQLAEVTLSLKACPPCPNS | 200 (199) |
| | ttgagcgtgaaagcttgtccaccc | | |
| | tgcccgaattct | | |
| H26 | cggattcaccagatgaactccgag | RIHQMNSELSVLANS | 202 (201) |
| | ttgagcgtgctcgcgaattct | | |
| H27 | cggattcaccagatgaactccgag | RIHQMNSELSVLACPPCPNS | 204 (203) |
| | ttgagcgtgctcgcttgtccaccc | | |
| | tgcccgaattct | | |
| H28 | gataccaaagggaagaacgtcctc | DTKGKNVLEKIFSNS | 206 (205) |
| | gagaagatcttctcgaattct | | |
| H29 | gataccaaagggaagaacgtcctc | DTKGKNVLEKIFDSCPPCPNS | 208 (207) |
| | gagaagatcttcgactcctgtcca | | |
| | ccttgcccgaattct | | |
| H30 | ctgccacctgagacacaggagagt | LPPETQESQEVTLNS | 210 (209) |
| | caagaagtcaccctgaattct | | |
| H31 | ctgccacctgagacacaggagagt | LPPETQESQEVTLSCPPCPNS | 212 (211) |
| | caagaagtcaccctgtcctgtcca | | |
| | ccttgcccgaattct | | |
| H32 | cggattcacctgaacgtgtccgag | RIHLNVSERPFPPNS | 214 (213) |
| | aggccctttcctccgaattct | | |
| H33 | cggattcacctgaacgtgtccgag | RIHLNVSERPFPPCPPCPNS | 216 (215) |
| | aggccctttcctccctgtccaccc | | |
| | tgcccgaattct | | |
| H36 | gggtgtccaccttgtccaggcggt | GCPPCPGGGGSNS | 222 (221) |
| | ggtggatcgaattct | | |
| H40 | ggatgtccaccttgtcccgcgaat | GCPPCPANS | 230 (229) |
| | tct | | |
| H44 | ggaggagctagttgtccaccttgt | GGASCPPCPGNS | 238 (237) |
| | cccgggaattct | | |
| H45 | ggaggagccagttgtccaccttgt | GGASCPPCAGNS | 240 (239) |
| | gccgggaattct | | |
| H46 | ggaggagccagttgtccaccttgt | GGASCPPCANS | 242 (241) |
| | gcgaattct | | |
| (G4S)3 | ggtggcggtggatccggcggaggt | GGGSGGGSGGGS | 245 (244) |
| | gggtcgggtggcggcggatct | | |
| (G4S)4 | ggtggcggtggctcgggcggtggt | GGGSGGGSGGGSGGGGS | 247 (246) |
| | ggatctggaggaggtgggagcggg | | |
| | ggaggtggcagt | | |

Alternative hinge and linker sequences that can be used as connecting regions may be crafted from portions of cell surface receptors that connect IgV-like or IgC-like domains. Regions between IgV-like domains where the cell surface receptor contains multiple IgV-like domains in tandem and between IgC-like domains where the cell surface receptor contains multiple tandem IgC-like regions could also be used as connecting regions or linker peptides. The preferred hinge and linker sequences are from 5 to 60 amino acids long, and may be primarily flexible, but may also provide more rigid characteristics, may contain primarily a helical structure with minimal β sheet structure. The preferred sequences are stable in plasma and serum and are resistant to proteolytic cleavage. The preferred sequences may contain a naturally occurring or added motif such as CPPC that confers the capacity to form a disulfide bond or multiple disulfide bonds to stabilize the C-terminus of the molecule. The preferred sequences may contain one or more glycosylation sites. Examples of preferred hinge and linker sequences include, but are not limited to, the interdomain regions between the IgV-like and IgC-like or between the IgC-like or IgV-like domains of CD2, CD4, CD22, CD33, CD48, CD58, CD66, CD80, CD86, CD96, CD150, CD166, and CD244. Alternative hinges may also be crafted from disulfide-containing regions of Type II receptors from non-IgSF members such as CD69, CD72 and CD161.

### PIMS Leader Peptide

PIMS leader peptides are designed to be used for their known purpose of functioning as a signal sequence to facilitate secretion of expressed PIMS molecules. Using any of the conventional leader peptides (signal sequences) is expected to direct nascently expressed PIMS molecules into a secretory pathway and to result in cleavage of the leader peptide from the mature PIMS molecule at or near the junction between the leader peptide and the PIMS molecule. A particular leader peptide will be chosen based on considerations known in the art, and sequences encoded by polynucleotides specifying restriction endonuclease cleavage sites may be introduced at the beginning and end of the coding sequence for the leader peptide to facilitate molecular engineering, provided that such introduced sequences specify amino acids that either do not interfere unacceptably with any desired processing of the leader peptide from the nascently expressed protein or do not interfere unacceptably with any desired function of the PIMS molecule if the leader peptide is not cleaved during maturation of the PIMS molecule. Exemplary leader peptides, used in the Examples, include the 2E12 or anti-CD28 antibody leader peptide disclosed herein, which has the amino acid sequence H₃N-MDFQVQIFSFLLISASVIMSRG-CO₂H (see, e.g., residues 1-22 of SEQ ID NO:2; see also, residues 1-23 of SEQ ID NO:4 which is the leader extended by a single V residue at the C-terminus), and the human VK3 leader peptide, H₃N-MEAPAQLLFLLLLWLPDTTG-CO₂H (SEQ ID NO:250; encoded, e.g., by nucleotides 1-60 of SEQ ID NO:358).

PIMS molecules exhibit a markedly different structure relative to other antibody and antibody-like binding proteins. A PIMS molecule has at least one specific binding domain, e.g., an scFv antigen-binding domain, that is located C-terminal to an immunoglobulin effector domain (i.e., a constant sub-region), such as an IgG1 effector domain. Also, the structure of a PIMS molecule is typically unique in that it has at least one region capable of participating in disulfide bond linkages, the PIMS linker region disposed between the effector and specific binding domains, which is always present, and the N-terminal hinge region that may be found in a PIMS molecule. Preferably, an N-terminal hinge region is derived from the same antibody class, isotype and sub-isotype as at least one of the C_{H2} and C_{H3} domains of that PIMS molecule. Further, the hinge or hinge-like domains of a PIMS molecule can be altered to affect either the effector domain, the binding domain, or both. In addition, PIMS molecules are expected to be easier to purify with minimal aggregation, which should give this molecular architecture a practical advantage as far as stability and amenability to production or manufacture.

There is a radical departure in design between PIMS molecules, placing the constant sub-region towards the N-terminal end of the polypeptide, and proteins, polypeptides and peptides found in nature. PIMS molecules, including both the PIMS polypeptides and their encoding nucleic acids or polynucleotides, exhibit a modular design adaptable to a wide variety of molecules. As noted herein, a PIMS molecule comprises a constant sub-region derived from an immunoglobulin, typically containing C_{H2} and C_{H3} domains derived from the same (preferred) or different antibodies, a PIMS linker peptide that may be a hinge region derived from an antibody, and a specific binding domain that comprises at least one binding region. Frequently, a mature PIMS molecule will further exhibit an N-terminal sequence derived from an antibody hinge region. Thus, a PIMS molecule according to the invention includes, but is not limited to, combinations of the modules (constant subregion, linker (hinge), and specific binding domain) disclosed herein or known in the art, located in relative orientation as provided herein. The invention expressly contemplates combining modules from the SMIP and Scorpion molecules identified in Table 5, with the sequence endpoints of the various modules noted in the Table.

TABLE 5

| | | Sequence Identifier (exemplary |
|---|---|---|
| Description | Features (amino acid endpoints) | encoding polynucleotide) |
| anti-CD20 (2H7) LH (AA) | Leader: 1-22 | 120 (119) |
| | VL: 23-128 | |
| | Linker: 129-144 | |
| | VH: 145-265 | |
| anti-CD28 (2e12) LH (AA) | Leader: 1-23 | 126 (125) |
| | VL: 24-135 | |
| | Linker: 136-150 | |
| | VH: 151-271 | |
| anti-CD28 (2e12) HL (AA) | Leader: 1-23 | 128 (127) |
| | VH: 24-144 | |
| | Linker: 145-164 | |
| | VL: 165-276 | |
| G-28-1 VLVH (AA) | VL: 1-107 | 130 (129) |
| | Linker: 108-124 | |
| | VH: 125-239 | |
| G28-1 VHVL (AA) | VH: 1-121 | 132 (131) |
| | Linker: 122-144 | |
| | VL: 145-253 | |
| G-19-4 VLVH (AA) | VL: 1-108 | 134 (133) |
| | Linker: 109-125 | |
| | VH: 126-247 | |
| G19-4 VHVL (AA) | VH: 1-122 | 136 (135) |
| | Linker: 123-139 | |
| | VL: 140-248 | |
| 019044 VHVL (AA) | | 138 (137) |
| 2H7sssIgG1-STD1-2e12HL | Leader: 1-22 | 2 (1) |
| (w/2E12 leader) (AA) C_{H2}C_{H3} | VL: 23-128 | |
| | Linker: 129-144 | |
| | VH: 145-265 | |
| | Hinge: 268-281 | |
| | EFD-BD2 linker: 500-519 | |
| | VH2: 520-640 | |
| | Linker2: 641-660 | |
| | VL2: 661-772 | |
| 2H7sssIgG1(P238S/P331S)- | Leader: 1-22 | 3 |
| STD1-2e12HL (w/2e12 | VL: 23-128 | |
| leader) (AA) | Linker: 129-144 | |
| C_{H2}C_{H3} | VH: 145-265 | |
| | Hinge: 268-282 | |
| | EFD-BD2 linker: 500-519 | |
| | VH2: 520-640 | |
| | Linker2: 641-660 | |
| | VL2: 661-772 | |
| 2H7sssIgG1-STD1-2e12LH | Leader: 1-22 | 5 (4) |
| (w/2e12 leader) (AA) | VL: 23-128 | |
| | Linker: 129-144 | |
| | VH: 145-265 | |
| | Hinge: 268-282 | |
| | EFD-BD2 Linker: 500-519 | |
| | VL2: 520-631 | |
| | Linker2: 632-646 | |
| | VH2: 647-767 | |
| 2H7sssIgG1(P238S/P331S)- | Leader: 1-22 | 6 |
| STD1-2e12LH (w/2e12 | VL: 23-128 | |
| leader) (AA) | Linker: 129-144 | |
| | VH: 145-265 | |
| | Hinge: 268-282 | |
| | EFD-BD2 Linker: 500-519 | |
| | VL2: 520-631 | |
| | Linker2: 632-646 | |
| | VH2: 647-767 | |
| 2H7sssIgG1-STD2-2e12LH | Leader: 1-22 | 8 (7) |
| (w/2e12 leader) (AA) | VL: 23-129 | |
| | Linker: 130-144 | |
| | VH: 145-265 | |
| | Hinge: 268-282 | |
| | EFD-BD2 Linker: 500-537 | |
| | VL2: 538-649 | |
| | Linker2: 650-664 | |
| | VH2: 665-785 | |
| 2H7sssIgG1(P238S/P331S)- | Leader: 1-22 | 9 |
| STD2-2e12LH (w/2e12 | VL: 23-128 | |
| leader) (AA) | Linker: 129-144 | |
| | VH: 145-265 | |
| | Hinge: 268-281 | |
| | EFD-BD2 Linker: 500-537 | |
| | VL2: 538-649 | |
| | Linker2: 650-664 | |
| | VH2: 665-785 | |
| 2H7sssIgG1-STD2-2e12HL | Leader: 1-22 | 11 (10) |
| (w/2e12 leader) (AA) | VL: 23-128 | |
| | Linker: 129-144 | |
| | VH: 145-265 | |
| | Hinge: 268-282 | |
| | EFD-BD2 Linker: 500-537 | |
| | VH2: 538-658 | |
| | Linker2: 659-678 | |
| | VL2: 679-790 | |
| 2H7sssIgG1(P238S/P331S)- | Leader: 1-22 | 12 |
| STD2-2e12HL (w/2e12 | VL: 23-128 | |
| leader) (AA) | Linker: 129-144 | |
| | VH: 145-265 | |
| | Hinge: 268-282 | |
| | EFD-BD2 Linker: 500-537 | |
| | VH2: 538-658 | |
| | Linker2: 659-678 | |
| | VL2: 679-790 | |
| 2H7sssIgG1-H1-2e12HL | Leader: 1-22 | 14 (13) |
| (w/2e12 leader) (AA) | VL: 23-128 | |
| | Linker: 129-144 | |
| | VH: 145-265 | |
| | Hinge: 268-281 | |
| | EFD-BD2 Linker: 500-501 | |
| | VH2: 502-622 | |
| | Linker2: 623-642 | |
| | VL2: 643-754 | |
| 2H7sssIgG1-H2-2e12HL | Leader: 1-22 | 16 (15) |
| (w/2e12 leader) (AA) | VL: 23-127 | |
| | Linker: 128-144 | |
| | VH: 145-265 | |
| | Hinge: 268-282 | |
| | EFD-BD2 Linker: 500-507 | |
| | VH2: 508-628 | |
| | Linker2: 629-648 | |
| | VL2: 649-760 | |
| 2H7sssIgG1-H3-2e12HL | Leader: 1-22 | 18 (17) |
| (w/e12 leader) (AA) | VL: 23-128 | |
| | Linker: 129-144 | |
| | VH: 145-265 | |
| | Hinge: 268-282 | |
| | EFD-BD2 Linker: 500-509 | |
| | VH2: 510-630 | |
| | Linker2: 631-650 | |
| | VL2: 651-762 | |
| 2H7sssIgG1-H4-2e12HL | Leader: 1-22 | 20 (19) |
| (AA) | VL: 23-128 | |
| | Linker: 129-144 | |
| | VH: 145-265 | |
| | Hinge: 268-282 | |
| | EFD-BD2 Linker: 500-512 | |
| | VH2: 513-633 | |
| | Linker2: 634-653 | |
| | VL2: 654-765 | |
| 2H7sssIgG1-H5-2e12HL | Leader: 1-22 | 22 (21) |
| (w/2e12 leader) (AA) | VL: 23-128 | |
| | Linker: 129-144 | |
| | VH: 145-265 | |
| | Hinge: 268-282 | |
| | EFD-BD2 Linker: 500-514 | |
| | VH2: 515-635 | |
| | Linker2: 636-655 | |
| | VL2: 656-767 | |
| 2H7sssIgG1-H6-2e12HL | Leader: 1-22 | 24 (23) |
| (w/2e12 leader) (AA) | VL: 23-128 | |
| | Linker: 129-144 | |
| | VH: 145-265 | |
| | Hinge: 268-282 | |
| | EFD-BD2 Linker: 500-517 | |
| | VH2: 518-638 | |
| | Linker2: 639-658 | |
| | VL2: 659-770 | |
| 2H7sscIgG1-H7-2e12HL | Leader: 1-22 | 26 (25) |
| (w/2e12 linker) (AA) | VL: 23-128 | |
| | Linker: 129-144 | |
| | VH: 145-265 | |
| | Hinge: 268-282 | |
| | EFD-BD2 Linker: 500-507 | |
| | VH2: 508-628 | |
| | Linker2: 629-648 | |
| | VL2: 649-760 | |
| 2H7sssIgG1-H7-G194 HL | Leader: 1-22 | 28 (27) |
| (w/2e12 leader) (AA) | VL1: 23-128 | |
| | Linker: 129-144 | |
| | VH1: 145-265 | |
| | Hinge: 268-282 | |
| | EFD-BD2 Linker: 500-507 | |
| | VH2: 508-629 | |
| | Linker2: 630-646 | |
| | VL2: 647-754 | |
| 2H7sssIgG1-H7-G281 HL | Leader: 1-22 | 30 (29) |
| (w/2e12 leader) (AA) | VL1: 23-128 | |
| | Linker: 129-144 | |
| | VH1: 145-265 | |
| | Hinge: 268-282 | |
| | EFD-BD2 Linker: 500-507 | |
| | VH2: 508-629 | |
| | Linker2: 630-651 | |
| | VL2: 652-759 | |
| 2e12-sss-IgG1 HL SMIP | Leader: 1-22 | 32 (31) |
| (AA) | VH: 24-144 | |
| | Linker: 145-164 | |
| | VL: 165-276 | |
| | Hinge: 279-293 | |
| 2e12-sss-IgG1 LH SMIP | Leader: 1-23 | 34 (33) |
| (AA) | VL: 24-135 | |
| | Linker: 136-150 | |
| | VH: 151-271 | |
| | Hinge: 274-288 | |
| G28-1 LH SMIP (AA) | Leader: 1-20 | 36 (35) |
| | VL: 21-127 | |
| | Linker: 128-144 | |
| | VH: 145-260 | |
| | Hinge: 261-275 | |
| G28-1 HL SMIP (AA) | Leader: 1-20 | 38 (37) |
| | VH: 21-136 | |
| | Linker: 137-158 | |
| | VL: 159-266 | |
| | Hinge: 268-283 | |
| G19-4 LH SMIP (AA) | Leader: 1-20 | 40 (39) |
| | VL: 21-128 | |
| | Linker: 129-145 | |
| | VH: 146-267 | |
| | Hinge: 268-282 | |
| G19-4 HL SMIP (AA) | Leader: 1-20 | 42 (41) |
| | VH: 21-142 | |
| | Linker: 143-159 | |
| | VL: 160-267 | |
| | Hinge: 270-284 | |
| n2H7sssIgG1-STD1-2e12HL | Leader: 1-20 | 44 (43) |
| (AA) | VL: 21-127 | |
| | Linker: 128-142 | |
| | VH: 143-264 | |
| | Hinge: 265-279 | |
| | EFD-BD2 Linker: 497-516 | |
| | VH2: 517-637 | |
| | Linker2: 638-657 | |
| | VL2: 658-769 | |
| n2H7sssIgG1-STD2-2e12LH | Leader: 1-20 | 46 (45) |
| (AA) | VL: 21-126 | |
| | Linker: 127-142 | |
| | VH: 143-264 | |
| | Hinge: 265-279 | |
| | EFD-BD2 Linker: 497-516 | |
| | VL2: 517-628 | |
| | Linker2: 629-643 | |
| | VH2: 644-764 | |
| n2H7sssIgG1-H1-2e12HL | Leader: 1-20 | 48 (47) |
| (AA) | VL: 21-126 | |
| | Linker: 127-142 | |
| | VH: 143-264 | |
| | Hinge: 265-279 | |
| | EFD-BD2 Linker: 497-498 | |
| | VH2: 499-619 | |
| | Linker2: 620-639 | |
| | VL2: 640-751 | |
| n2H7sssIgG1-H2-2e12HL | Leader: 1-20 | 50 (49) |
| (AA) | VL: 21-126 | |
| | Linker: 127-142 | |
| | VH: 143-264 | |
| | Hinge: 265-279 | |
| | EFD-BD2 Linker: 497-504 | |
| | VH2: 505-625 | |
| | Linker2: 626-645 | |
| | VL2: 646-757 | |
| n2H7sssIgG1-H3-2e12HL | Leader: 1-20 | 52 (51) |
| (AA) | VL: 21-126 | |
| | Linker: 127-142 | |
| | VH: 143-264 | |
| | Hinge: 265-279 | |
| | EFD-BD2 Linker: 497-506 | |
| | VH2: 507-627 | |
| | Linker2: 628-647 | |
| | VL2: 648-759 | |
| n2H7sssIgG1-H4-2e12HL | Leader: 1-20 | 54 (53) |
| (AA) | VL: 21-126 | |
| | Linker: 127-142 | |
| | VH: 143-264 | |
| | Hinge: 265-279 | |
| | EFD-BD2 Linker: 497-509 | |
| | VH2: 510-630 | |
| | Linker2: 631-650 | |
| | VL2: 651-762 | |
| n2H7sssIgG1-H5-2e12HL | Leader: 11-20 | 56 (55) |
| (AA) | VL: 21-126 | |
| | Linker: 127-142 | |
| | VH: 143-264 | |
| | Hinge: 265-279 | |
| | EFD-BD2 Linker: 497-511 | |
| | VH2: 512-632 | |
| | Linker2: 633-652 | |
| | VL2: 653-764 | |
| n2H7sssIgG1-H6-2e12HL | Leader: 1-20 | 58 (57) |
| (AA) | VL: 21-126 | |
| | Linker: 127-142 | |
| | VH: 143-264 | |
| | Hinge: 265-279 | |
| | EFD-BD2 Linker: 497-514 | |
| | VH2: 515-635 | |
| | Linker2: 636-655 | |
| | VL2: 656-767 | |
| 2H7cscIgG1-STD1-2e12HL | Leader: 1-22 | 60 (59) |
| (w/2E12 leader) (AA) | VL: 23-128 | |
| | Linker: 129-144 | |
| | VH: 145-265 | |
| | Hinge: 268-282 | |
| | EFD-BD2 Linker: 500-519 | |
| | VH2: 520-640 | |
| | Linker2: 641-660 | |
| | VL2: 661-772 | |

One use for PIMS molecules is in the assessment and/or optimization of the effector domain and/or the specific binding domain of, e.g., multi-specific binding proteins such as scorpions. These PIMS molecules have activity on their own as well as offering a platform for better assessing Scorpion Binding Domain 2 (BD2) and the effector domain-BD2 Scorpion linker. Beyond the uses as specific binding proteins with effector function and the uses in assessing Scorpion BD2 and effector domain-BD2 linker activities, PIMS molecules may be modified to alter, e.g., an effector domain activity such as ADCC and/or CDC activities, which might include changes to either the N-terminal hinge (e.g., IgG1 SCC-P hinge) or the hinge-like PIMS linker disposed between the constant sub-region or effector domain and the specific binding domain/(s). Data obtained to date show that the ADCC activity of humanized CD20 PIMS are at least as good as their SMIP counterparts and have the potential to be potent alternative molecules for delivering enhanced effector functions to specific targets, such as cells displaying CD20.

### Proteins and Polypeptides

In certain embodiments of the invention, there are provided any of the herein-described specific binding proteins with effector function, or PIMS, wherein the specific binding protein or peptide with effector function comprises two or more binding domain polypeptide sequences (e.g., a V_{L} and a V_{H}) constituting at least one specific binding site. The binding domain polypeptide sequence is derived from an antigen variable region. The antibodies from which the binding domains are derived may be antibodies that are polyclonal, including monospecific polyclonal, monoclonal (mAbs), recombinant, chimeric, humanized (such as CDR-grafted), human, single-chain, catalytic, and any other form of antibody known in the art, as well as fragments, variants or derivatives thereof. In some embodiments, a binding domain is a binding site (e.g., a camelid binding domain). In some embodiments, each of the binding domains of the protein according to the invention is derived from a complete variable region of an immunoglobulin. In preferred embodiments, the binding domains are each based on a human Ig variable region. In other embodiments, the protein is derived from a fragment of an Ig variable region. In such embodiments, it is preferred that each binding domain polypeptide sequence correspond to the sequences of each of the complementarity determining regions of a given Ig variable region. Also contemplated within the invention are binding domains that correspond to fewer than all CDRs of a given Ig variable region, provided that such binding domains retain the capacity to specifically bind to at least one target.

The specific binding protein with effector function or PIMS also has a constant sub-region sequence derived from an immunoglobulin constant region, preferably an antibody heavy chain constant region, covalently linked through its C-terminus to a PIMS linker region, the PIMS linker region in turn being linked through its C-terminus to a binding domain in the PIMS molecule.

In some embodiments, the PIMS linker interposed between the constant sub-region and a binding domain is derived from a wild-type hinge region of an immunoglobulin, such as an IgG1, IgG2, IgG3, IgG4, IgA, IgD or an IgE hinge region. In other embodiments, the invention provides PIMS with PIMS linkers that are derived from altered hinges. One category of altered hinge regions suitable for inclusion in PIMS molecules is the category of hinges with an altered number of cysteine residues, particularly those Cys residues known in the art to be involved in interchain disulfide bond formation in immunoglobulin counterpart molecules having wild-type hinges. Thus, proteins may have an IgG1 hinge in which one of the three hinge Cys residues capable of participating in interchain disulfide bond formations is missing. To indicate the cysteine sub-structure of altered hinges, the Cys subsequence is presented from N- to C-terminus. Using this identification system, the PIMS with altered IgG hinges include hinge structures characterized as cxc, xxc, ccx, xxc, xcx, cxx, and xxx, where “x” is “not c”. The Cys residue may be either deleted or substituted by an amino acid that results in a conservative substitution or a non-conservative substitution. In some embodiments, the cysteine is replaced by a serine.

For PIMS proteins with IgG1 hinges or hinge-like structures, there may be 0, 1, 2, or 3 Cys residues in the PIMS linker or N-terminal hinge region, and preferably 1 or 2 Cys residues. For proteins with IgG2 hinges, there may be 0, 1, 2, 3, or 4 Cys residues, preferably 1 or 2 Cys residues. For altered IgG2 hinges containing 1, 2 or 3 Cys residues, all possible subsets of Cys residues are contemplated. Thus, for IgG2 hinges having one Cys, the PIMS molecule may have the following Cys motif in the hinge region: cxxx, xcxx, xxcx, or xxxc. For IgG2 hinge variants having 2 or 3 Cys residues, all possible combinations of retained and substituted (or deleted) Cys residues are contemplated. For PIMS with altered IgG3 or altered IgG4 hinge regions, a reduction in Cys residues from 1 to the complete number of Cys residues in the hinge region is contemplated, regardless of whether the loss is through deletion or substitution by conservative or non-conservative amino acids (e.g., Serine). In like manner, PIMS having a wild-type IgA, IgD or IgE hinge are contemplated, as are corresponding altered hinge regions having a reduced number of Cys residues extending from 0 to the total number of Cys residues found in the corresponding wild-type hinge. In some embodiments having an IgG1 hinge, the first, or N-terminal, Cys residue of the hinge is retained. It is contemplated that PIMS will be capable of forming homo-multimers, such as homo-dimers. Further, proteins with altered hinges may have alterations at the termini of the hinge region, e.g., loss or substitution of one or more amino acid residues at the N-terminus, C-terminus or both termini of a given region or domain, such as a PIMS linker or hinge domain, as disclosed herein.

In another exemplary embodiment, the constant sub-region is derived from a constant region that comprises a native, or an engineered, IgD hinge region. The wild-type human IgD hinge has one cysteine that forms a disulfide bond with the light chain in the native IgD structure. In some embodiments, this IgD hinge cysteine is mutated (e.g., deleted) to generate an altered hinge for use as a PIMS linker region between the constant sub-region and a specific binding domain. Other amino acid changes or deletions or alterations in an IgD hinge that do not result in undesired hinge inflexibility are within the scope of the invention. Native or engineered IgD hinge regions from other species are also within the scope of the invention, as are humanized native or engineered IgD hinges from non-human species, and (other non IgD) hinge regions from other human, or non-human, antibody isotypes, (such as the llama IgG2 hinge).

The invention further comprehends constant sub-regions attached at the C-terminus, and optionally at the N-terminus, to hinges and PIMS linkers that correspond to a known hinge region, such as an IgG1 hinge or an IgD hinge, as noted above. The hinge may be a modified or altered (relative to wild-type) hinge region in which at least one cysteine residue known to participate in inter-chain disulfide bond linkage is replaced by another amino acid in a conservative substitution (e.g., Ser for Cys) or a non-conservative substitution.

Alternative hinge and PIMS linker sequences that can be used as connecting regions are from portions of cell surface receptors that connect immunoglobulin V-like or immunoglobulin C-like domains. Regions between Ig V-like domains where the cell surface receptor contains multiple Ig V-like domains in tandem, and between Ig C-like domains where the cell surface receptor contains multiple tandem Ig C-like regions are also contemplated as connecting regions. Hinge and PIMS linker sequences are typically from 5 to 60 amino acids long, and may be primarily flexible, but may also provide more rigid characteristics. In addition, PIMS linkers frequently provide spacing that facilitates minimization of steric hindrance between the binding domains. Preferably, these hinge and PIMS linker peptides are primarily a helical in structure, with minimal β sheet structure. The preferred sequences are stable in plasma and serum and are resistant to proteolytic cleavage. The preferred sequences may contain a naturally occurring or added motif such as the CPPC motif that confers a disulfide bond to stabilize dimer formation. The preferred sequences may contain one or more glycosylation sites. Examples of preferred hinge and PIMS linker sequences include, but are not limited to, the interdomain regions between the Ig V-like and Ig C-like regions of CD2, CD4, CD22, CD33, CD48, CD58, CD66, CD80, CD86, CD150, CD166, and CD244.

The constant sub-region may be derived from a camelid constant region, such as either a llama or camel IgG2 or IgG3. Specifically contemplated is a constant sub-region having the C_{H2}-C_{H3}, or hinge—C_{H2}-C_{H3}, region from any Ig class, or from any IgG subclass, such as IgG1 (e.g., human IgG1). The constant sub-region also may be a C_{H3} domain from any Ig class or subclass, such as IgG1 (e.g., human IgG1).

IgA constant domains, such as an IgA1 hinge, an IgA2 hinge, an IgA C_{H2} and an IgA C_{H3} domains with a mutated or missing tailpiece are also contemplated as constant sub-regions. The constant sub-region may also correspond to engineered antibodies in which, e.g., a loop graft has been constructed by making selected amino acid substitutions using an IgG framework to generate a binding site for a receptor other than a natural F_{C}R (CD16, CD32, CD64, F_{C}εR1), as would be understood in the art. An exemplary constant sub-region of this type is an IgG C_{H2}-C_{H3} region modified to have a CD89 binding site.

This aspect of the invention provides a specific binding protein or peptide having effector function, comprising, consisting essentially of, or consisting of (a) an N-terminally disposed constant sub-region polypeptide binding domain polypeptide sequence derived from an immunoglobulin constant region that is fused or otherwise connected to (b) a PIMS linker region sequence, wherein the PIMS linker region polypeptide may be as described herein, and may comprise, consist essentially of, or consist of, for example, a hinge region or an alternative hinge region polypeptide sequence, in turn fused or otherwise connected to (c) a C-terminally disposed native or engineered binding domain polypeptide sequence derived from an immunoglobulin.

The constant sub-region polypeptide sequence derived from an immunoglobulin constant region is capable of at least one immunological activity selected from the group consisting of antibody dependent cell-mediated cytotoxicity, CDC, complement fixation, and Fc receptor binding, and the binding domain polypeptide is capable of specifically binding to a target, such as an antigen.

This aspect of the invention also comprehends variant proteins or polypeptides exhibiting an effector function that are at least 80%, and preferably 85%, 90%, 95%, 99%, or 99.5% identical to a specific binding protein with effector function of specific sequence as disclosed herein.

### Polynucleotides

The invention also provides polynucleotides (isolated or purified or pure polynucleotides) encoding the proteins or peptides according to the invention, vectors (including cloning vectors and expression vectors) comprising such polynucleotides, and cells (e.g., host cells) transformed or transfected with a polynucleotide or vector according to the invention. In encoding the proteins or polypeptides of the invention, the polynucleotides encode a constant sub-region (an Fc domain), a PIMS linker, and a specific binding domain, all derived from immunoglobulins, preferably human immunoglobulins. The binding domain may contain a sequence corresponding to a full-length variable region sequence (either heavy chain and/or light chain), or to a partial sequence thereof, provided that each such binding domain retains the capacity to specifically bind. The constant sub-region or Fc domain may have a sequence that corresponds to a full-length immunoglobulin Fc domain sequence or to a partial sequence thereof, provided that the Fc domain exhibits at least one effector function as defined herein, and provided that the Fc domain is not a complete antibody Fc region. In addition, each of the binding domains of a given binding site may be joined via a linker peptide that typically is at least 8, and preferably at least 13, amino acids in length. A preferred linker sequence is a sequence based on the Gly4Ser motif, such as (Gly4Ser)ₙ, were n=3-5.

Variants of the specific binding proteins with effector function are also comprehended by the invention. Variant polynucleotides are at least 90%, and preferably 95%, 99%, or 99.9% identical to one of the polynucleotides of defined sequence as described herein, or that hybridize to one of those polynucleotides of defined sequence under stringent hybridization conditions of 0.015 M sodium chloride, 0.0015 M sodium citrate at 65-68° C. or 0.015 M sodium chloride, 0.0015M sodium citrate, and 50% formamide at 42° C. The polynucleotide variants retain the capacity to encode a specific binding protein with effector function or PIMS.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989).

More stringent conditions (such as higher temperature, lower ionic strength, higher formamide, or other denaturing agent) may also be used; however, the rate of hybridization will be affected. In instances wherein hybridization of deoxyoligonucleotides is concerned, additional exemplary stringent hybridization conditions include washing in 6×SSC, 0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

In a related aspect of the invention, there is provided a method of producing a polypeptide or protein or other construct of the invention, for example, including a PIMS, comprising the steps of (a) culturing a host cell as described or provided for herein under conditions that permit expression of the construct; and (b) isolating the PIMS expression product from the host cell or host cell culture.

### Constructs

The present invention also relates to vectors, and to constructs prepared from known vectors, that each include a polynucleotide or nucleic acid of the invention, and in particular to recombinant expression constructs, including any of various known constructs, including delivery constructs, useful for gene therapy, that include any nucleic acids encoding, for example, PIMS, as provided herein; to host cells which are genetically engineered with vectors and/or other constructs of the invention and to methods of administering expression or other constructs comprising nucleic acid sequences encoding a PIMS, or fragments or variants thereof, by recombinant techniques.

Various constructs of the invention encoding PIMS can be expressed in virtually any host cell, including in vivo host cells in the case of use for gene therapy, under the control of appropriate promoters, depending on the nature of the construct (e.g., type of promoter, as described above), and depending on the nature of the desired host cell (e.g., postmitotic terminally differentiated or actively dividing; e.g., maintenance of an expressible construct as an episome or integrated into the host cell genome).

Appropriate cloning and expression vectors for use with prokaryotic and eukaryotic hosts are described, for example, in Sambrook, et al., Molecular Cloning: A Laboratory Manual, Second Edition, Cold Spring Harbor, N.Y., (1989). Exemplary cloning/expression vectors include, but are not limited to, cloning vectors, shuttle vectors, and expression constructs, that may be based on plasmids, phagemids, phasmids, cosmids, viruses, artificial chromosomes, or any nucleic acid vehicle suitable for amplification, transfer, and/or expression of a polynucleotide contained therein that is known in the art. As noted herein, in preferred embodiments of the invention, recombinant expression is conducted in mammalian cells that have been transfected, transformed or transduced with a nucleic acid according to the invention. See also, for example, Machida, C A., “Viral Vectors for Gene Therapy: Methods and Protocols”; Wolff, J A, “Gene Therapeutics: Methods and Applications of Direct Gene Transfer” (Birkhäuser 1994); Stein, U and Walther, W (eds., “Gene Therapy of Cancer: Methods and Protocols” (Humana Press 2000); Robbins, P D (ed.), “Gene Therapy Protocols” (Humana Press 1997); Morgan, J R (ed.), “Gene Therapy Protocols” (Humana Press 2002); Meager, A (ed.), “Gene Therapy Technologies, Applications and Regulations: From Laboratory to Clinic” (John Wiley & Sons Inc. 1999); MacHida, C A and Constant, J G, “Viral Vectors for Gene Therapy: Methods and Protocols” (Humana Press 2002); “New Methods Of Gene Therapy For Genetic Metabolic Diseases NIH Guide,” Volume 22, Number 35, Oct. 1, 1993. See also U.S. Pat. Nos. 6,384,210; 6,384,203; 6,384,202; 6,384,018; 6,383,814; 6,383,811; 6,383,795; 6,383,794; 6,383,785; 6,383,753; 6,383,746; 6,383,743; 6,383,738; 6,383,737; 6,383,733; 6,383,522; 6,383,512; 6,383,481; 6,383,478; 6,383,138; 6,380,382; 6,380,371; 6,380,369; 6,380,362; 6,380,170; 6,380,169; 6,379,967; and 6,379,966.

Typically, expression constructs are derived from plasmid vectors. One preferred construct is a modified pNASS vector (Clontech, Palo Alto, Calif.), which has nucleic acid sequences encoding an ampicillin resistance gene, a polyadenylation signal and a T7 promoter site. Other suitable mammalian expression vectors are well known (see, e.g., Ausubel et al., 1995; Sambrook et al., supra; see also, e.g., catalogues from Invitrogen, San Diego, Calif.; Novagen, Madison, Wis.; Pharmacia, Piscataway, N.J.). Presently preferred constructs may be prepared that include a dihydrofolate reductase (DHFR)-encoding sequence under suitable regulatory control, for promoting enhanced production levels of PIMS, which levels result from gene amplification following application of an appropriate selection agent (e.g., methotrexate).

Generally, recombinant expression vectors will include origins of replication and selectable markers permitting transformation of the host cell, and a promoter derived from a highly expressed gene to direct transcription of a downstream structural sequence, as described above. A vector in operable linkage with a polynucleotide according to the invention yields a cloning or expression construct. Exemplary cloning/expression constructs contain at least one expression control element, e.g., a promoter, operably linked to a polynucleotide of the invention. Additional expression control elements, such as enhancers, factor-specific binding sites, terminators, and ribosome binding sites are also contemplated in the vectors and cloning/expression constructs according to the invention. The heterologous structural sequence of the polynucleotide according to the invention is assembled in appropriate phase with translation initiation and termination sequences. Thus, for example, the PIMS-encoding nucleic acids as provided herein may be included in any one of a variety of expression vector constructs as a recombinant expression construct for expressing such a protein in a host cell. In certain preferred embodiments, the constructs are included in formulations that are administered in vivo. Such vectors and constructs include chromosomal, nonchromosomal and synthetic DNA sequences, e.g., derivatives of SV40; bacterial plasmids; phage DNA; yeast plasmids; vectors derived from combinations of plasmids and phage DNA; viral DNA, such as vaccinia, adenovirus, fowl pox virus, and pseudorabies; or replication deficient retroviruses as described below. However, any other vector may be used for preparation of a recombinant expression construct, and in preferred embodiments such a vector will be replicable and viable in the host.

The appropriate DNA sequence(s) may be inserted into a vector, for example, by a variety of procedures. In general, a DNA sequence is inserted into an appropriate restriction endonuclease cleavage site(s) by procedures known in the art. Standard techniques for cloning, DNA isolation, amplification and purification, for enzymatic reactions involving DNA ligase, DNA polymerase, restriction endonucleases and the like, and various separation techniques are contemplated. A number of standard techniques are described, for example, in Ausubel et al. (1993 Current Protocols in Molecular Biology, Greene Publ. Assoc. Inc. & John Wiley & Sons, Inc., Boston, Mass.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.); Glover (Ed.) (1985 DNA Cloning Vol. I and II, IRL Press, Oxford, UK); Hames and Higgins (Eds.), (1985 Nucleic Acid Hybridization, IRL Press, Oxford, UK); and elsewhere.

The DNA sequence in the expression vector is operatively linked to at least one appropriate expression control sequence (e.g., a constitutive promoter or a regulated promoter) to direct mRNA synthesis. Representative examples of such expression control sequences include promoters of eukaryotic cells or their viruses, as described above. Promoter regions can be selected from any desired gene using CAT (chloramphenicol acetyltransferase) vectors or other vectors with selectable markers. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art, and preparation of certain particularly preferred recombinant expression constructs comprising at least one promoter or regulated promoter operably linked to a nucleic acid encoding a protein or polypeptide according to the invention is described herein.

Transcription of the DNA encoding proteins and polypeptides of the invention by higher eukaryotes may be increased by inserting an enhancer sequence into the vector. Examples include the SV40 enhancer on the late side of the replication origin bp 100 to 270, a cytomegalovirus early promoter enhancer, the polyoma enhancer on the late side of the replication origin, and adenovirus enhancers.

Gene therapies using the nucleic acids of the invention are also contemplated, comprising strategies to replace defective genes or add new genes to cells and/or tissues, and is being developed for application in the treatment of cancer, the correction of metabolic disorders and in the field of immunotherapy. Gene therapies of the invention include the use of various constructs of the invention, with or without a separate carrier or delivery vehicle or constructs, for treatment of the diseases, disorders, and/or conditions noted herein. Such constructs may also be used as vaccines for treatment or prevention of the diseases, disorders, and/or conditions noted herein. DNA vaccines, for example, make use of polynucleotides encoding immunogenic protein and nucleic acid determinants to stimulate the immune system against pathogens or tumor cells. Such strategies can stimulate either acquired or innate immunity or can involve the modification of immune function through cytokine expression. In vivo gene therapy involves the direct injection of genetic material into a patient or animal, typically to treat, prevent or ameliorate a disease or symptoms associated with a disease. Vaccines and immune modulation are systemic therapies. With tissue-specific in vivo therapies, such as those that aim to treat cancer, localized gene delivery and/or expression/targeting systems are preferred. Diverse gene therapy vectors that target specific tissues are known in the art, and procedures have been developed to physically target specific tissues, for example, using catheter-based technologies, all of which are contemplated herein.

Ex vivo approaches to gene therapy are also contemplated herein and involve the removal, genetic modification, expansion and re-administration of a subject's, e.g., human patient's, own cells. Examples include bone marrow transplantation for cancer treatment or the genetic modification of lymphoid progenitor cells. Ex vivo gene therapy is preferably applied to the treatment of cells that are easily accessible and can survive in culture during the gene transfer process (such as blood or skin cells).

Useful gene therapy vectors include adenoviral vectors, lentiviral vectors, Adeno-associated virus (AAV) vectors, Herpes Simplex Virus (HSV) vectors, and retroviral vectors. Gene therapies may also be carried out using “naked DNA,” liposome-based delivery, lipid-based delivery (including DNA attached to positively charged lipids), electroporation, and ballistic projection.

In certain embodiments, including but not limited to gene therapy embodiments, the vector may be a viral vector such as, for example, a retroviral vector. Miller et al., 1989 BioTechniques 7:980; Coffin and Varmus, 1996 Retroviruses, Cold Spring Harbor Laboratory Press, NY. For example, retroviruses from which the retroviral plasmid vectors may be derived include, but are not limited to, Moloney Murine Leukemia Virus, spleen necrosis virus, retroviruses such as Rous Sarcoma Virus, Harvey Sarcoma virus, avian leukosis virus, gibbon ape leukemia virus, human immunodeficiency virus, adenovirus, Myeloproliferative Sarcoma Virus, and mammary tumor virus.

Retroviruses are RNA viruses which can replicate and integrate into the genome of a host cell via a DNA intermediate. This DNA intermediate, or provirus, may be stably integrated into the host cell DNA. According to certain embodiments of the present invention, an expression construct may comprise a retrovirus into which a foreign gene that encodes a foreign protein is incorporated in place of normal retroviral RNA. When retroviral RNA enters a host cell coincident with infection, the foreign gene is also introduced into the cell, and may then be integrated into host cell DNA as if it were part of the retroviral genome. Expression of this foreign gene within the host results in expression of the foreign protein.

Most retroviral vector systems that have been developed for gene therapy are based on murine retroviruses. Such retroviruses exist in two forms, as free viral particles referred to as virions, or as proviruses integrated into host cell DNA. The virion form of the virus contains the structural and enzymatic proteins of the retrovirus (including the enzyme reverse transcriptase), two RNA copies of the viral genome, and portions of the source cell plasma membrane containing viral envelope glycoprotein. The retroviral genome is organized into four main regions: the Long Terminal Repeat (LTR), which contains cis-acting elements necessary for the initiation and termination of transcription and is situated both 5′ and 3′ to the coding genes, and the three genes encoding gag, pol, and env. These three genes, gag, pol, and env, encode, respectively, internal viral structures, enzymatic proteins (such as integrase), and the envelope glycoprotein (designated gp70 and p15e) which confers infectivity and host range specificity of the virus, as well as the “R” peptide of undetermined function.

Separate packaging cell lines and vector-producing cell lines have been developed because of safety concerns regarding the uses of retroviruses, including uses in expression constructs. Briefly, this methodology employs the use of two components, a retroviral vector and a packaging cell line (PCL). The retroviral vector contains long terminal repeats (LTRs), the foreign DNA to be transferred and a packaging sequence (y). This retroviral vector will not reproduce by itself because the genes which encode structural and envelope proteins are not included within the vector genome. The PCL contains genes encoding the gag, pol, and env proteins, but does not contain the packaging signal “y.” Thus, a PCL can only form empty virion particles by itself. Within this general method, the retroviral vector is introduced into the PCL, thereby creating a vector-producing cell line (VCL). This VCL manufactures virion particles containing only the foreign genome of the retroviral vector, and therefore has previously been considered to be a safe retrovirus vector for therapeutic use.

A “retroviral vector construct” refers to an assembly which, in preferred embodiments of the invention, is capable of directing the expression of a sequence(s) or gene(s) of interest, such as a PIMS-encoding nucleic acid sequence. Briefly, the retroviral vector construct must include a 5′ LTR, a tRNA binding site, a packaging signal, an origin of second-strand DNA synthesis and a 3′ LTR. A wide variety of heterologous sequences may be included within the vector construct including, for example, sequences which encode a protein (e.g., cytotoxic protein, disease-associated antigen, immune accessory molecule, or replacement protein), or which are useful as a molecule itself (e.g., as a ribozyme or antisense sequence).

Retroviral vector constructs of the present invention may be readily constructed from a wide variety of retroviruses, including for example, B, C, and D type retroviruses as well as spumaviruses and lentiviruses (see, e.g., RNA Tumor Viruses, Second Edition, Cold Spring Harbor Laboratory, 1985). Such retroviruses may be readily obtained from depositories or collections such as the American Type Culture Collection (“ATCC”; Rockville, Md.), or isolated from known sources using commonly available techniques. Any of the above retroviruses may be readily utilized in order to assemble or construct retroviral vector constructs, packaging cells, or producer cells of the invention, given the disclosure provided herein and standard recombinant techniques (e.g., Sambrook et al, Molecular Cloning: A Laboratory Manual, 2d ed., Cold Spring Harbor Laboratory Press, 1989; Kunkle, 1985 Proc. Natl. Acad. Sci. (USA) 82:488).

Suitable promoters for use in viral vectors generally may include, but are not limited to, the retroviral LTR; the SV40 promoter; and the human cytomegalovirus (CMV) promoter described in Miller, et al., 1989 Biotechniques 7:980 990, or any other promoter (e.g., cellular promoters such as eukaryotic cellular promoters including, but not limited to, the histone, pol III, and β-actin promoters). Other viral promoters that may be employed include, but are not limited to, adenovirus promoters, thymidine kinase (TK) promoters, and B19 parvovirus promoters. The selection of a suitable promoter will be apparent to those skilled in the art from the teachings contained herein, and may be from among either regulated promoters or promoters as described above.

The retroviral plasmid vector is employed to transduce packaging cell lines to form producer cell lines. Examples of packaging cells which may be transfected include, but are not limited to, the PE501, PA317, ψ-2, ψ-AM, PA12, T19-14X, VT-19-17-H2, ψCRE, ψCRIP, GP+E-86, GP+envAm12, and DAN cell lines as described in Miller, Human Gene Therapy, 1:5-14 (1990). The vector may transduce the packaging cells through any means known in the art. Such means include, but are not limited to, electroporation, the use of liposomes, and Ca.PO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the PIMS. Such retroviral vector particles then may be employed to transduce eukaryotic cells, either in vitro or in vivo. The transduced eukaryotic cells will express the nucleic acid sequence(s) encoding the protein or polypeptide. Eukaryotic cells that may be transduced include, but are not limited to, embryonic stem cells, as well as hematopoietic stem cells, hepatocytes, fibroblasts, circulating peripheral blood mononuclear and polymorphonuclear cells including myelomonocytic cells, lymphocytes, myoblasts, tissue macrophages, dendritic cells, Kupffer cells, lymphoid and reticuloendothelial cells of the lymph nodes and spleen, keratinocytes, endothelial cells, and bronchial epithelial cells.

### Host Cells

A further aspect of the invention provides a host cell transformed or transfected with, or otherwise containing, any of the polynucleotides or cloning/expression constructs of the invention. The polynucleotides and cloning/expression constructs are introduced into suitable cells using any method known in the art, including transformation, transfection and transduction. Host cells include the cells of a subject undergoing ex vivo cell therapy including, for example, ex vivo gene therapy. Eukaryotic host cells contemplated as an aspect of the invention when harboring a polynucleotide, vector, or protein according to the invention include, in addition to a subject's own cells (e.g., a human patient's own cells), VERO cells, HeLa cells, Chinese hamster ovary (CHO) cell lines (including modified CHO cells capable of modifying the glycosylation pattern of expressed PIMS, see Published US Patent Application No. 2003/0115614 A1), incorporated herein by reference, COS cells (such as COS-7), W138, BHK, HepG2, 3T3, RIN, MDCK, A549, PC12, K562, HEK293 cells, HepG2 cells, N cells, 3T3 cells, Spodoptera frugiperda cells (e.g., Sf9 cells), Saccharomyces cerevisiae cells, and any other eukaryotic cell known in the art to be useful in expressing, and optionally isolating, a protein or peptide according to the invention. Also contemplated are prokaryotic cells, including but not limited to, Escherichia coli, Bacillus subtilis, Salmonella typhimurium, a Streptomycete, or any prokaryotic cell known in the art to be suitable for expressing, and optionally isolating, a protein or peptide according to the invention. In isolating protein or peptide from prokaryotic cells, in particular, it is contemplated that techniques known in the art for extracting protein from inclusion bodies may be used. The selection of an appropriate host is within the scope of those skilled in the art from the teachings herein.

The engineered host cells can be cultured in a conventional nutrient medium modified as appropriate for activating promoters, selecting transformants, or amplifying particular genes. The culture conditions for particular host cells selected for expression, such as temperature, pH and the like, will be readily apparent to the ordinarily skilled artisan. Various mammalian cell culture systems can also be employed to express recombinant protein. Examples of mammalian expression systems include the COS-7 lines of monkey kidney fibroblasts, described by Gluzman, 1981 Cell 23:175, and other cell lines capable of expressing a compatible vector, for example, the C127, 3T3, CHO, HeLa and BHK cell lines. Mammalian expression vectors will comprise an origin of replication, a suitable promoter, and optionally, enhancer, and also any necessary ribosome binding sites, polyadenylation site, splice donor and acceptor sites, transcriptional termination sequences, and 5′ flanking nontranscribed sequences, for example as described herein regarding the preparation of PIMS expression constructs. DNA sequences derived from the SV40 splice, and polyadenylation sites may be used to provide the required nontranscribed genetic elements. Introduction of the construct into the host cell can be effected by a variety of methods with which those skilled in the art will be familiar, including but not limited to, calcium phosphate transfection, DEAE-Dextran-mediated transfection, or electroporation (Davis et al., 1986 Basic Methods in Molecular Biology).

In one embodiment, a host cell is transduced by a recombinant viral construct directing the expression of a protein or polypeptide according to the invention. The transduced host cell produces viral particles containing expressed protein or polypeptide derived from portions of a host cell membrane incorporated by the viral particles during viral budding.

### Pharmaceutical Compositions

In some embodiments, the compositions of the invention, such as a PIMS or a composition comprising a polynucleotide encoding such a protein as described herein, are suitable to be administered under conditions and for a time sufficient to permit expression of the encoded protein in a host cell in vivo or in vitro, for gene therapy, and the like. Such compositions may be formulated into pharmaceutical compositions for administration according to well known methodologies. Pharmaceutical compositions generally comprise one or more recombinant expression constructs, and/or expression products of such constructs, in combination with a pharmaceutically acceptable carrier, excipient or diluent. Such carriers will be nontoxic to recipients at the dosages and concentrations employed. For nucleic acid-based formulations, or for formulations comprising expression products according to the invention, about 0.01 μg/kg to about 100 mg/kg body weight will be administered, for example, by the intradermal, subcutaneous, intramuscular or intravenous route, or by any route known in the art to be suitable under a given set of circumstances. A preferred dosage, for example, is about 1 μg/kg to about 1 mg/kg, with about 5 μg/kg to about 200 μg/kg particularly preferred.

It will be evident to those skilled in the art that the number and frequency of administration will be dependent upon the response of the host. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate buffered saline at physiological pH may be used. Preservatives, stabilizers, dyes and the like may be provided in the pharmaceutical composition. For example, sodium benzoate, sorbic acid and esters of p-hydroxybenzoic acid may be added as preservatives. Id. at 1449. In addition, antioxidants and suspending agents may be used. Id. The compounds of the present invention may be used in either the free base or salt forms, with both forms being considered as being within the scope of the present invention.

The pharmaceutical compositions that contain one or more nucleic acid constructs of the invention, or the proteins corresponding to the products encoded by such nucleic acid constructs, may be in any form which allows for the composition to be administered to a patient. For example, the composition may be in the form of a solid, liquid or gas (aerosol). Typical routes of administration include, without limitation, oral, topical, parenteral, buccal, sublingual, rectal, vaginal, and intranasal. The term parenteral as used herein includes subcutaneous injections, intravenous, intramuscular, intrasternal, intracavernous, intrathecal, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, preferred compositions contain, in addition to one or more PIMS construct or expressed product, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following compounds: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates, agents for the adjustment of tonicity such as sodium chloride or dextrose, and any adjuvant known in the art. Examples of immunostimulatory substances (adjuvants) include N-acetylmuramyl-L-alanine-D-isoglutamine (MDP), lipopolysaccharides (LPS), glucan, IL 12, GM CSF, gamma interferon and IL 15. The parenteral preparation can be enclosed in ampoules, disposable syringes or multiple-dose vials made of glass or plastic. An injectable pharmaceutical composition is preferably sterile.

It may also be desirable to include other components in the preparation, such as delivery vehicles including, but not limited to, aluminum salts, water-in-oil emulsions, biodegradable oil vehicles, oil-in-water emulsions, biodegradable microcapsules, and liposomes. Examples of immunostimulatory substances (adjuvants) for use in such vehicles are identified above.

While any suitable carrier known to those of ordinary skill in the art may be employed in the pharmaceutical compositions of this invention, the type of carrier will vary depending on the mode of administration and whether a sustained release is desired. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109, incorporated herein by reference. In this regard, it is preferable that the microsphere be larger than approximately 25 microns.

Pharmaceutical compositions may also contain diluents such as buffers, antioxidants such as ascorbic acid, low molecular weight (less than about 10 residues) polypeptides, proteins, amino acids, carbohydrates (e.g., glucose, sucrose or dextrins), chelating agents (e.g., EDTA), glutathione and other stabilizers and excipients. Neutral buffered saline or saline mixed with nonspecific serum albumin are exemplary appropriate diluents. Preferably, product is formulated as a lyophilizate using appropriate excipient solutions (e.g., sucrose) as diluents.

The pharmaceutical compositions according to the invention also include stabilized proteins and stable liquid pharmaceutical formulations in accordance with technology known in the art, including the technology disclosed in Published US Patent Application No. 2006/0008415 A1, incorporated herein by reference. Such technologies include derivatization of a protein, wherein the protein comprises a thiol group coupled to N-acetyl-L-cysteine, N-ethyl-maleimide, or cysteine.

As described above, the subject invention includes compositions capable of delivering nucleic acid molecules encoding PIMS molecules. Such compositions include recombinant viral vectors, e.g., retroviruses (see WO 90/07936, WO 91/02805, WO 93/25234, WO 93/25698, and WO 94/03622), adenovirus (see Berkner, 1988 Biotechniques 6:616-627; Li et al., 1993 Hum. Gene Ther. 4:403-409; Vincent et al., Nat. Genet. 5:130-134; and Kolls et al., 1994 Proc. Natl. Acad. Sci. USA 91:215-219), pox virus (see U.S. Pat. No. 4,769,330; U.S. Pat. No. 5,017,487; and WO 89/01973)), recombinant expression construct nucleic acid molecules complexed to a polycationic molecule (see WO 93/03709), and nucleic acids associated with liposomes (see Wang et al., 1987 Proc. Natl. Acad. Sci. USA 84:7851). In certain embodiments, the DNA may be linked to killed or inactivated adenovirus (see Curiel et al., 1992 Hum. Gene Ther. 3:147-154; Cotton et al., 1992 Proc. Natl. Acad. Sci. USA 89:6094). Other suitable compositions include DNA-ligand (see Wu et al., 1989 J. Biol. Chem. 264:16985-16987) and lipid-DNA combinations (see Felgner et al., 1989 Proc. Natl. Acad. Sci. USA 84:7413-7417).

In addition to direct in vivo procedures, ex vivo procedures may be used in which cells are removed from a host (e.g., a subject, such as a human patient), modified, and placed into the same or another host animal. It will be evident that one can utilize any of the compositions noted above for introduction of constructs of the invention, either the proteins/polypeptides or the nucleic acids encoding them into tissue cells in an ex vivo context. Protocols for viral, physical and chemical methods of uptake are well known in the art.

### Generation of Antibodies

Polyclonal antibodies directed toward an antigen polypeptide generally are produced in animals (e.g., rabbits, hamsters, goats, sheep, horses, pigs, rats, gerbils, guinea pigs, mice, or any other suitable mammal, as well as other non-mammal species) by means of multiple subcutaneous or intraperitoneal injections of antigen polypeptide or a fragment thereof and an adjuvant. Adjuvants include, but are not limited to, complete or incomplete Freund's adjuvant, mineral gels such as aluminum hydroxide, and surface active substances such as lysolecithin, pluronic polyols, polyanions, peptides, oil emulsions, and dinitrophenol. BCG (bacilli Calmette-Guerin) and Corynebacterium parvum are also potentially useful adjuvants. It may be useful to conjugate an antigen polypeptide to a carrier protein that is immunogenic in the species to be immunized; typical carriers include keyhole limpet hemocyanin, serum albumin, bovine thyroglobulin, or soybean trypsin inhibitor. Also, aggregating agents such as alum are used to enhance the immune response. After immunization, the animals are bled and the serum is assayed for anti-antigen polypeptide antibody titer using conventional techniques. Polyclonal antibodies may be utilized in the sera from which they were detected, or may be purified from the sera using, e.g., antigen affinity chromatography.

Monoclonal antibodies directed toward antigen polypeptides are produced using any method which provides for the production of antibody molecules by continuous cell lines in culture. For example, monoclonal antibodies may be made by the hybridoma method as described in Kohler et al., Nature 256:495 [975]; the human B-cell hybridoma technique (Kosbor et al., Immunol Today 4:72, 1983; Cote et al., Proc Natl Acad Sci 80: 2026-2030, 1983) and the EBV-hybridoma technique (Cole et al., Monoclonal Antibodies and Cancer Therapy, Alan R Liss Inc, New York N.Y., pp 77-96, (1985).

When the hybridoma technique is employed, myeloma cell lines may be used. Cell lines suited for use in hybridoma-producing fusion procedures preferably do not produce endogenous antibody, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

In an alternative embodiment, human antibodies can be produced from phage-display libraries (Hoogenboom et al., J. Mol. Biol. 227: 381 [1991]; Marks et al., J. Mol. Biol. 222: 581, see also U.S. Pat. No. 5,885,793).). These processes mimic immune selection through the display of antibody repertoires on the surface of filamentous bacteriophage, and subsequent selection of phage by their binding to an antigen of choice. One such technique is described in PCT Application No. PCT/US98/17364, filed in the name of Adams et al., which describes the isolation of high affinity and functional agonistic antibodies for MPL- and msk-receptors using such an approach. In this approach, a complete repertoire of human antibody genes can be created by cloning naturally rearranged human V genes from peripheral blood lymphocytes as previously described (Mullinax, et al., Proc. Natl. Acad. Sci. (USA) 87: 8095-8099 [1990]).

Alternatively, an entirely synthetic human heavy chain repertoire can be created from unrearranged V gene segments by assembling each human V_{H} segment with D segments of random nucleotides together with a human J segment (Hoogenboom, et al., J. Mol. Biol. 227:381-388 [1992]). Likewise, a light chain repertoire can be constructed by combining each human V segment with a J segment (Griffiths, et al, EMBO J. 13:3245-3260). Nucleotides encoding the complete antibody (i.e., both heavy and light chains) are linked as a single chain Fv fragment and this polynucleotide is ligated to a nucleotide encoding a filamentous phage minor coat protein. When this fusion protein is expressed on the surface of the phage, a polynucleotide encoding a specific antibody can be identified by selection using an immobilized antigen.

Beyond the classic methods of generating polyclonal and monoclonal antibodies, any method for generating any known antibody form is contemplated. In addition to polyclonals and monoclonals, antibody forms include chimerized antibodies, humanized antibodies, CDR-grafted antibodies, and antibody fragments and variants.

### Variants and Derivatives of Specific Binding Agents

In one example, insertion variants are provided wherein one or more amino acid residues supplement the sequence of a specific binding domain. Insertions may be located at either or both termini of the protein, or may be positioned within internal regions of the specific binding domain. Variant products of the invention also include mature PIMS wherein leader or signal sequences are removed, with the resulting protein having additional amino terminal residues. The additional amino terminal residues may be derived from another protein, or may include one or more residues that are not identifiable as being derived from a specific protein. Polypeptides with an additional methionine residue at position −1 (e.g., Met-1-PIMS) are contemplated, as are polypeptides of the invention with additional methionine and lysine residues at positions −2 and −1 (Met-2-Lys-1-PIMS). The parenthetical designations emphasize the feature being described (i.e., N-terminal residues), and are not meant to define PIMS as molecules lacking such N-termini. Each of Met-1-PIMS and MET-2-Lys-1-PIMS are, in fact, PIMS. Variants of the polypeptides of the invention having additional Met, Met-Lys, or Lys residues (or one or more basic residues in general) are particularly useful for enhanced recombinant protein production in bacterial host cells.

The invention also embraces specific polypeptides of the invention having additional amino acid residues which arise from use of specific expression systems. For example, use of commercially available vectors that express a desired polypeptide as part of a glutathione-5-transferase (GST) fusion product provides the desired polypeptide having an additional glycine residue at position −1 after cleavage of the GST component from the desired polypeptide. Variants which result from expression in other vector systems are also contemplated, including those wherein histidine tags are incorporated into the amino acid sequence, generally at the carboxy and/or amino terminus of the sequence.

In another aspect, the invention provides deletion variants wherein one or more amino acid residues in a polypeptide of the invention are removed. Deletions can be effected at one or both termini of the polypeptide, or by removal of one or more residues from within the amino acid sequence. Deletion variants necessarily include all fragments of a polypeptide according to the invention.

Antibody fragments refer to polypeptides having a sequence corresponding to at least part of an immunoglobulin variable region sequence. Fragments may be generated, for example, by enzymatic or chemical cleavage of polypeptides corresponding to full-length antibodies. Other binding fragments include those generated by synthetic techniques or by recombinant DNA techniques, such as the expression of recombinant plasmids containing nucleic acid sequences encoding partial antibody variable regions. Preferred polypeptide fragments display immunological properties unique to, or specific for, a target as described herein. Fragments of the invention having the desired immunological properties can be prepared by any of the methods well known and routinely practiced in the art.

In still another aspect, the invention provides substitution variants of PIMS. Substitution variants include those polypeptides wherein one or more amino acid residues in an amino acid sequence are removed and replaced with alternative residues. In some embodiments, the substitutions are conservative in nature; however, the invention embraces substitutions that ore also non-conservative. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table A (see WO 97/09433, page 10, published Mar. 13, 1997 (PCT/GB96/02197, filed Sep. 6, 1996), immediately below.

TABLE A
Conservative Substitutions I
SIDE CHAIN CHARACTERISTIC AMINO ACID

| | | |
|---|---|---|
| Aliphatic | Non-polar | G A P I L V |
| | Polar - uncharged | S T M N Q |
| | Polar - charged | D E K R |
| Aromatic | | H F W Y |
| Other | | N Q D E |

Alternatively, conservative amino acids can be grouped as described in Lehninger, [Biochemistry, Second Edition; Worth Publishers, Inc. NY:NY (1975), pp. 71-77] as set out in Table B, immediately below.

TABLE B
Conservative Substitutions II

| | SIDE CHAIN | |
|---|---|---|
| TYPE | CHARACTERISTIC | AMINO ACID |
| Non-polar | A. Aliphatic: | A L I V P |
| (hydrophobic) | B. Aromatic | F W |
| | C. Sulfur-containing | M |
| | D. Borderline | G |
| Uncharged-polar | A. Hydroxyl | S T Y |
| | B. Amides | N Q |
| | C. Sulfhydryl | C |
| | D. Borderline | G |
| Positively Charged | | K R H |
| (Basic) | | |
| Negatively Charged | | D E |
| (Acidic) | | |

| | | |
|---|---|---|
| SIDE CHAIN | CHARACTERISTIC | AMINO ACID |

| | | |
|---|---|---|
| Non-polar | A. Aliphatic: | A L I V P |
| (hydrophobic) | B. Aromatic: | F W |
| | C. Sulfur-containing: | M |
| | D. Borderline: | G |
| Uncharged-polar | A. Hydroxyl: | S T Y |
| | B. Amides: | N Q |
| | C. Sulfhydryl: | C |
| | D. Borderline: | G |
| Positively Charged | | K R H |
| (Basic) | | |
| Negatively Charged | | D E |
| (Acidic) | | |

The invention also provides derivatives of PIMS polypeptides. Derivatives include PIMS polypeptides bearing modifications other than insertion, deletion, or substitution of amino acid residues. Preferably, the modifications are covalent in nature and include, for example, chemical bonding with polymers, lipids, other organic, and inorganic moieties. Derivatives of the invention may be prepared to increase circulating half-life of a specific PIMS polypeptide, or may be designed to improve targeting capacity for the polypeptide to desired cells, tissues, or organs.

The invention further embraces PIMS that are covalently modified or derivatized to include one or more water-soluble polymer attachments such as polyethylene glycol, polyoxyethylene glycol, or polypropylene glycol, as described U.S. Pat. Nos. 4,640,835, 4,496,689, 4,301,144, 4,670,417, 4,791,192 and 4,179,337. Still other useful polymers known in the art include monomethoxy-polyethylene glycol, dextran, cellulose, and other carbohydrate-based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers. Particularly preferred are polyethylene glycol (PEG)-derivatized proteins. Water-soluble polymers may be bonded at specific positions, for example at the amino terminus of the proteins and polypeptides according to the invention, or randomly attached to one or more side chains of the polypeptide. The use of PEG for improving therapeutic capacities is described in U.S. Pat. No. 6,133,426 to Gonzales, et al.

### Target Sites for Immunoglobulin Mutagenesis

Certain strategies are available to manipulate inherent properties of an antigen-specific immunoglobulin (e.g., an antibody) that are not available to non-immunoglobulin-based binding molecules. A good example of the strategies favoring, e.g., antibody-based molecules, over these alternatives is the in vivo modulation of the affinity of an antibody for its target through affinity maturation, which takes advantage of the somatic hypermutation of immunoglobulin genes to yield antibodies of increasing affinity as an immune response progresses. Additionally, recombinant technologies have been developed to alter the structure of immunoglobulins and immunoglobulin regions and domains. Thus, polypeptides derived from antibodies may be produced that exhibit altered affinity for a given antigen, and a number of purification protocols and monitoring screens are known in the art for identifying and purifying or isolating these polypeptides. Using these known techniques, polypeptides comprising antibody-derived binding domains can be obtained that exhibit decreased or increased affinity for an antigen. Strategies for generating the polypeptide variants exhibiting altered affinity include the use of site-specific or random mutagenesis of the DNA encoding the antibody to change the amino acids present in the protein, followed by a screening step designed to recover antibody variants that exhibit the desired change, e.g., increased or decreased affinity relative to the unmodified parent or referent antibody.

The amino acid residues most commonly targeted in mutagenic strategies to alter affinity are those in the complementarity-determining region (CDR) or hyper-variable region of the light and the heavy chain variable regions of an antibody. These regions contain the residues that physicochemically interact with an antigen, as well as other amino acids that affect the spatial arrangement of these residues. However, amino acids in the framework regions of the variable domains outside the CDR regions have also been shown to make substantial contributions to the antigen-binding properties of an antibody, and can be targeted to manipulate such properties. See Hudson, P. J. Curr. Opin. Biotech., 9: 395-402 (1999) and references therein.

Smaller and more effectively screened libraries of antibody variants can be produced by restricting random or site-directed mutagenesis to sites in the CDRs that correspond to areas prone to “hyper-mutation” during the somatic affinity maturation process. See Chowdhury, et al., Nature Biotech., 17: 568-572 (1999) and references therein. The types of DNA elements known to define hyper-mutation sites in this manner include direct and inverted repeats, certain consensus sequences, secondary structures, and palindromes. The consensus DNA sequences include the tetrabase sequence Purine-G-Pyrimidine-A/T (i.e., A or G-G-C or T-A or T) and the serine codon AGY (wherein Y can be C or T).

Thus, another aspect of the invention is a set of mutagenic strategies for modifying the affinity of an antibody for its target. These strategies include mutagenesis of the entire variable region of a heavy and/or light chain, mutagenesis of the CDR regions only, mutagenesis of the consensus hypermutation sites within the CDRs, mutagenesis of framework regions, or any combination of these approaches (“mutagenesis” in this context could be random or site-directed). Definitive delineation of the CDR regions and identification of residues comprising the binding site of an antibody can be accomplished though solving the structure of the antibody in question, and the antibody:ligand complex, through techniques known to those skilled in the art, such as X-ray crystallography. Various methods based on analysis and characterization of such antibody crystal structures are known to those of skill in the art and can be employed to approximate the CDR regions. Examples of such commonly used methods include the Kabat, Chothia, AbM and contact definitions.

The Kabat definition is based on sequence variability and is the most commonly used definition to predict CDR regions. Johnson, et al., Nucleic Acids Research, 28: 214-8 (2000). The Chothia definition is based on the location of the structural loop regions. (Chothia et al., J. Mol. Biol., 196: 901-17 [1986]; Chothia et al., Nature, 342: 877-83 [1989].) The AbM definition is a compromise between the Kabat and Chothia definitions. AbM is an integral suite of programs for antibody structure modeling produced by the Oxford Molecular Group (Martin, et al., Proc. Natl. Acad. Sci. (USA) 86:9268-9272 [1989]; Rees, et al., ABMTM, a computer program for modeling variable regions of antibodies, Oxford, UK; Oxford Molecular, Ltd.). The AbM suite models the tertiary structure of an antibody from primary sequence using a combination of knowledge databases and ab initio methods An additional definition, known as the contact definition, has been recently introduced. See MacCallum et al., J. Mol. Biol., 5:732-45 (1996). This definition is based on an analysis of the available complex crystal structures.

By convention, the CDR domains in the heavy chain are typically referred to as H1, H2 and H3, and are numbered sequentially in order moving from the amino terminus to the carboxy terminus. The CDR regions in the light chain are typically referred to as L1, L2 and L3, and are numbered sequentially in order moving from the amino terminus to the carboxy terminus.

The CDR-H1 is approximately 10 to 12 residues in length and typically starts 4 residues after a Cys according to the Chothia and AbM definitions, or typically 5 residues later according to the Kabat definition. The H1 is typically followed by a Trp, typically Trp-Val, but also Trp-Ile, or Trp-Ala. The length of H1 is approximately 10 to 12 residues according to the AbM definition, while the Chothia definition excludes the last 4 residues.

The CDR-H2 typically starts 15 residues after the end of H1 according to the Kabat and AbM definitions. The residues preceding H2 are typically Leu-Glu-Trp-Ile-Gly but there are a number of variations. H2 is typically followed by the amino acid sequence Lys/Arg-Leu/Ile/Val/Phe/Thr/Ala-Thr/Ser/Ile/Ala. According to the Kabat definition, the length of H2 is approximately 16 to 19 residues, where the AbM definition predicts the length to be typically 9 to 12 residues.

The CDR-H3 typically starts 33 residues after the end of H2 and is typically preceded by the amino acid sequence Cys-Ala-Arg. H3 is typically followed by the amino acid Gly. The length of H3 ranges from 3 to 25 residues.

The CDR-L1 typically starts at approximately residue 24 and will typically follow a Cys. The residue after the CDR-L1 is always Trp and will typically begin one of the following sequences: Trp-Tyr-Gln, Trp-Leu-Gln, Trp-Phe-Gln, or Trp-Tyr-Leu. The length of CDR-L1 is approximately 10 to 17 residues.

The CDR-L2 starts approximately 16 residues after the end of L1. It will generally follow residues Ile-Tyr, Val-Tyr, Ile-Lys or Ile-Phe. The length of CDR-L2 is approximately 7 residues.

The CDR-L3 typically starts 33 residues after the end of L2 and typically follows a Cys. L3 is typically followed by the amino acid sequence Phe-Gly-XXX-Gly. The length of L3 is approximately 7 to 11 residues.

Various methods for modifying antibodies have been described in the art, including, e.g., methods of producing humanized antibodies wherein the sequence of the humanized immunoglobulin heavy chain variable region framework is 65% to 95% identical to the sequence of the donor immunoglobulin heavy chain variable region framework. Each humanized immunoglobulin chain will usually comprise, in addition to the CDRs, amino acids from the donor immunoglobulin framework that are, e.g., capable of interacting with the CDRs to affect binding affinity, such as one or more amino acids that are immediately adjacent to a CDR in the donor immunoglobulin or those within about 3 angstroms, as predicted by molecular modeling. The heavy and light chains may each be designed by using any one or all of various position criteria. When combined into an intact antibody, humanized immunoglobulins are substantially non-immunogenic in humans and retain substantially the same affinity as the donor immunoglobulin to the antigen, such as a protein or other compound containing the relevant epitope.

In one example, methods for the production of antibodies, and antibody fragments, are described that have binding specificity similar to a parent antibody, but which have increased human characteristics. Humanized antibodies are obtained by chain shuffling using, for example, phage display technology and a polypeptide comprising the heavy or light chain variable region of a non-human antibody specific for an antigen of interest, which is then combined with a repertoire of human complementary (light or heavy) chain variable regions. Hybrid pairings which are specific for the antigen of interest are identified and human chains from the selected pairings are combined with a repertoire of human complementary variable domains (heavy or light). In another embodiment, a component of a CDR from a non-human antibody is combined with a repertoire of component parts of CDRs from human antibodies. From the resulting library of antibody polypeptide dimers, hybrids are selected and may be used in a second humanizing shuffling step; alternatively, this second step is eliminated if the hybrid is already of sufficient human character to be of therapeutic value. Methods of modification to increase human character are known in the art.

Another example is a method for making humanized antibodies by substituting a CDR amino acid sequence for the corresponding human CDR amino acid sequence and/or substituting a FR amino acid sequence for the corresponding human FR amino acid sequences.

Yet another example provides methods for identifying the amino acid residues of an antibody variable domain that may be modified without diminishing the native affinity of the antigen binding domain while reducing its immunogenicity with respect to a heterologous species and methods for preparing these modified antibody variable regions as useful for administration to heterologous species.

Modification of an immunoglobulin such as an antibody by any of the methods known in the art is designed to achieve increased or decreased binding affinity for an antigen and/or to reduce immunogenicity of the antibody in the recipient and/or to modulate effector activity levels. In one approach, humanized antibodies can be modified to eliminate glycosylation sites in order to increase affinity of the antibody for its cognate antigen (Co, et al., Mol. Immunol. 30:1361-1367 [1993]). Techniques such as “reshaping,” hyperchimerization,” and “veneering/resurfacing” have produced humanized antibodies with greater therapeutic potential. Vaswami, et al., Annals of Allergy, Asthma, & Immunol 81:105 (1998); Roguska, et al., Prot. Engineer. 9:895-904 (1996)]. See also U.S. Pat. No. 6,072,035, which describes methods for reshaping antibodies. While these techniques diminish antibody immunogenicity by reducing the number of foreign residues, they do not prevent anti-idiotypic and anti-allotypic responses following repeated administration of the antibodies. Alternatives to these methods for reducing immunogenicity are described in Gilliland et al., J. Immunol. 62(6):3663-71 (1999).

In many instances, humanizing antibodies results in a loss of antigen binding capacity. It is therefore preferable to “back mutate” the humanized antibody to include one or more of the amino acid residues found in the original (most often rodent) antibody in an attempt to restore binding affinity of the antibody. See, for example, Saldanha et al., Mol. Immunol. 36:709-19 (1999).

Glycosylation of immunoglobulins has been shown to affect effector functions, structural stability, and the rate of secretion from antibody-producing cells (see Leatherbarrow et al., Mol. Immunol. 22:407 (1985), incorporated herein by reference). The carbohydrate groups responsible for these properties are generally attached to the constant regions of antibodies. For example, glycosylation of IgG at Asn 297 in the C_{H2} domain facilitates full capacity of the IgG to activate complement-dependent cytolysis (Tao et al., J. Immunol. 143:2595 (1989)). Glycosylation of IgM at Asn 402 in the C_{H3} domain, for example, facilitates proper assembly and cytolytic activity of the antibody (Muraoka et al., J. Immunol. 142:695 (1989)). Removal of glycosylation sites at positions 162 and 419 in the C_{H1} and C_{H3} domains of an IgA antibody led to intracellular degradation and at least 90% inhibition of secretion (Taylor et al., Wall, Mol. Cell. Biol. 8:4197 (1988)). Accordingly, the molecules of the invention include mutationally altered immunoglobulins exhibiting altered glycosylation patterns by mutation of specific residues in, e.g., a constant sub-region to alter effector function. See Co et al., Mol. Immunol. 30:1361-1367 (1993), Jacquemon et al., J. Thromb. Haemost. 4:1047-1055 (2006), Schuster et al., Cancer Res. 65:7934-7941 (2005), and Warnock et al., Biotechnol Bioeng. 92:831-842 (2005), each incorporated herein by reference.

The invention also includes PIMS having at least one binding domain that is at least 80%, preferably 90% or 95% or 99% identical in sequence to a known immunoglobulin variable region sequence and which has at least one residue that differs from such immunoglobulin variable region, wherein the changed residue adds a glycosylation site, changes the location of one or more glycosylation site(s), or preferably removes a glycosylation site relative to the immunoglobulin variable region. In some embodiments, the change removes an N-linked glycosylation site in a an immunoglobulin variable region framework, or removes an N-linked glycosylation site that occurs in the immunoglobulin heavy chain variable region framework in the region spanning about amino acid residue 65 to about amino acid residue 85, using the numbering convention of Co et al., J. Immunol. 148: 1149, (1992).

Any method known in the art is contemplated for producing the PIMS exhibiting altered glycosylation patterns relative to an immunoglobulin referent sequence. For example, any of a variety of genetic techniques may be employed to alter one or more particular residues. Alternatively, the host cells used for production may be engineered to produce the altered glycosylation pattern. One method known in the art, for example, provides altered glycosylation in the form of bisected, non-fucosylated variants that increase ADCC. The variants result from expression in a host cell containing an oligosaccharide-modifying enzyme. Alternatively, the Potelligent technology of BioWa/Kyowa Hakko is contemplated to reduce the fucose content of glycosylated molecules according to the invention. In one known method, a CHO host cell for recombinant immunoglobulin production is provided that modifies the glycosylation pattern of the immunoglobulin Fc region, through production of GDP-fucose. This technology is available to modify the glycosylation pattern of a constant sub-region of a PIMS according to the invention.

In addition to modifying the binding properties of binding domains, such as the binding domains of immunoglobulins, and in addition to such modifications as humanization, the invention comprehends the modulation of effector function by changing or mutating residues contributing to effector function, such as the effector function of an immunoglobulin constant sub-region. These modifications can be effected using any technique known in the art, such as the approach disclosed in Presta et al., Biochem. Soc. Trans. 30:487-490 (2001), incorporated herein by reference. Exemplary approaches would include the use of the protocol disclosed in Presta et al. to modify specific residues known to affect binding of one or more constant sub-regions to FCγRI, FCγRII, FCγRIII, FCαR, and/or FCεR.

In another approach, the Xencor XmAb technology is available to engineer constant sub-regions corresponding to Fc domains to enhance cell killing effector function. See Lazar et al., Proc. Natl. Acad. Sci. (USA) 103(11):4005-4010 (2006), incorporated herein by reference. Using this approach, for example, one can generate constant sub-regions optimized for FCγR specificity and binding, thereby enhancing cell killing effector function.

### Production of PIMS

A variety of expression vector/host systems may be used to contain and express the PIMS of the invention. These systems include, but are not limited to, microorganisms such as bacteria transformed with recombinant bacteriophage, plasmid, cosmid, or other expression vectors; yeast transformed with yeast expression or shuttle vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant PIMS productions include, but are not limited to, VERO cells, HeLa cells, Chinese hamster ovary (CHO) cells, COS cells (such as COS-7), W138, BHK, HepG2, 3T3, RIN, MDCK, A549, PC12, K562 and HEK293 cells. Exemplary protocols for the recombinant expression of PIMS are described hereinbelow.

An expression vector can comprise a transcriptional unit comprising an assembly of (1) a genetic element or elements having a regulatory role in gene expression, for example, a promoter, enhancer, or factor-specific binding site, (2) a structural sequence that encodes the PIMS which is transcribed into mRNA and translated into protein, and (3) appropriate transcription and translation initiation and termination sequences. Structural units intended for use in yeast or eukaryotic expression systems preferably include a leader sequence enabling extracellular secretion of translated protein by a host cell. Alternatively, where a recombinant PIMS is expressed without a leader or transport sequence, it may include an amino terminal methionine residue. This residue may or may not be subsequently cleaved from the expressed recombinant protein to provide a final PIMS.

For example, the PIMS may be recombinantly expressed in yeast using a commercially available expression system, e.g., the Pichia Expression System (Invitrogen, San Diego, Calif.), following the manufacturer's instructions. This system also relies on the pre-pro-alpha sequence to direct secretion, but transcription of the insert is driven by the alcohol oxidase (AOX1) promoter upon induction by methanol. The secreted PIMS peptide may be purified from the yeast growth medium by, e.g., the methods used to purify the peptide from bacterial and mammalian cell supernatants.

Alternatively, the cDNA encoding the PIMS peptide may be cloned into the baculovirus expression vector pVL1393 (PharMingen, San Diego, Calif.). This vector can be used according to the manufacturer's directions (PharMingen) to infect Spodoptera frugiperda cells in SF9 protein-free medium and to produce recombinant protein. The PIMS protein can be purified and concentrated from the medium using a heparin-Sepharose column (Pharmacia, Piscataway, N.J.). Insect systems for protein expression, such as the SF9 system, are well known to those of skill in the art. In one such system, Autographa californica nuclear polyhedrosis virus (AcNPV) can be used as a vector to express foreign genes in the Spodoptera frugiperda cells or in Trichoplusia larvae. The PIMS peptide coding sequence can be cloned into a nonessential region of the virus, such as the polyhedrin gene, and placed under control of the polyhedrin promoter. Successful insertion of the PIMS peptide will render the polyhedrin gene inactive and produce recombinant virus lacking coat protein. The recombinant viruses can be used to infect S. frugiperda cells or Trichoplusia larvae in which peptide is expressed (Smith et al., J Virol 46: 584, 1983; Engelhard et al., Proc Nat Acad Sci (USA) 91: 3224-7, 1994).

In another example, the DNA sequence encoding the PIMS peptide can be amplified by PCR and cloned into an appropriate vector, for example, pGEX-3× (Pharmacia, Piscataway, N.J.). The pGEX vector is designed to produce a fusion protein comprising glutathione-5-transferase (GST), encoded by the vector, and a PIMS protein encoded by a DNA fragment inserted into the cloning site of the vector. The primers for the PCR can be generated to include for example, an appropriate cleavage site to facilitate appropriate cloning. Where the PIMS protein fusion moiety is used solely to facilitate expression or is otherwise not desirable as an attachment to the peptide of interest, the recombinant PIMS protein fusion may then be cleaved from the GST portion of the fusion protein. The pGEX-3×/PIMS peptide construct is transformed into E. coli XL-1 Blue cells (Stratagene, La Jolla Calif.), and individual transformants isolated and grown. Plasmid DNA from individual transformants is purified and may be partially sequenced using an automated sequencer to confirm the presence of the desired PIMS protein-encoding nucleic acid insert in the proper orientation.

The fused PIMS protein, which may be produced as an insoluble inclusion body in the bacteria, can be purified as follows. Host cells can be harvested by centrifugation; washed in 0.15 M NaCl, 10 mM Tris, pH 8, 1 mM EDTA; and treated with 0.1 mg/ml lysozyme (Sigma Chemical Co.) for 15 minutes at room temperature. The lysate can be cleared by sonication, and cell debris can be pelleted by centrifugation for 10 minutes at 12,000 g. The fusion PIMS protein-containing pellet can be resuspended in 50 mM Tris, pH 8, and 10 mM EDTA, layered over 50% glycerol, and centrifuged for 30 minutes at 6000 g. The pellet can be resuspended in standard phosphate buffered saline solution (PBS) free of Mg++ and Ca++. The PIMS protein fusion can be further purified by fractionating the resuspended pellet in a denaturing SDS polyacrylamide gel (Sambrook et al.). The gel is soaked in 0.4 M KCl to visualize the protein, which is excised and electroeluted in gel-running buffer lacking SDS. If the GST/PIMS peptide fusion protein is produced in bacteria as a soluble protein, it can be purified using the GST Purification Module (Pharmacia Biotech).

The PIMS protein fusion is preferably subjected to digestion to cleave the GST from the PIMS peptide of the invention. The digestion reaction (20-40 μg fusion protein, 20-30 units human thrombin (4000 U/mg (Sigma) in 0.5 ml PBS) can be incubated 16-48 hours at room temperature and loaded on a denaturing SDS-PAGE gel to fractionate the reaction products. The gel can be soaked in 0.4 M KCl to visualize the protein bands. The identity of the protein band corresponding to the expected molecular weight of the PIMS peptide can be confirmed by amino acid sequence analysis using an automated sequencer (Applied Biosystems Model 473A, Foster City, Calif.). Alternatively, the identity can be confirmed by performing HPLC and/or mass spectrometry of the peptides.

Alternatively, a DNA sequence encoding the PIMS peptide can be cloned into a plasmid containing a desired promoter and, optionally, a leader sequence (see, e.g., Better et al., Science, 240:1041-43, 1988). The sequence of this construct can be confirmed by automated sequencing. The plasmid can then be transformed into a suitable E. coli strain, such as strain MC1061, using standard procedures employing CaCl₂ incubation and heat shock treatment of the bacteria (Sambrook et al.). The transformed bacteria can be grown in LB medium supplemented with carbenicillin or another suitable form of selection as would be known in the art, and production of the expressed protein can be induced by growth in a suitable medium. If present, the leader sequence can effect secretion of the PIMS peptide and be cleaved during secretion. The secreted recombinant protein can be purified from the bacterial culture medium by the methods described hereinbelow.

Mammalian host systems for the expression of the recombinant protein are well known to those of skill in the art and are preferred systems. Host cell strains can be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Different host cells such as CHO, HeLa, MDCK, 293, WI38, and the like, have specific cellular machinery and characteristic mechanisms for such post-translational activities and can be chosen to ensure the correct modification and processing of the foreign protein.

It is preferable that the transformed cells be used for long-term, high-yield protein production and, as such, stable expression is desirable. Once such cells are transformed with vectors that preferably contain at least one selectable marker along with the desired expression cassette, the cells are grown for 1-2 days in an enriched medium before being switched to selective medium. The selectable marker is designed to confer resistance to selection and its presence allows growth and recovery of cells that successfully express the foreign protein. Resistant clumps of stably transformed cells can be proliferated using tissue culture techniques appropriate to the cell.

A number of selection systems can be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk−, hgprt− or aprt− cells, respectively. Also, anti-metabolite resistance can be used as the basis of selection for dhfr, which confers resistance to methotrexate; gpt, which confers resistance to mycophenolic acid; neo, which confers resistance to the aminoglycoside G418 and confers resistance to chlorsulfuron; and hygro, which confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, β-glucuronidase and its substrate, GUS, and luciferase and its substrate, luciferin.

### Purification of Proteins

Protein purification techniques are well known to those of skill in the art. These techniques involve, at one level, the crude fractionation of the polypeptide and non-polypeptide fractions. Having separated the PIMS polypeptide from at least one other protein, the PIMS polypeptide is purified, but further purification using chromatographic, electrophoretic, and/or other known techniques to achieve partial or complete purification (or purification to homogeneity) is frequently desired. Analytical methods particularly suited to the preparation of a pure PIMS peptide are ion-exchange chromatography, exclusion chromatography; polyacrylamide gel electrophoresis; and isoelectric focusing. Particularly efficient methods of purifying peptides are fast protein liquid chromatography and HPLC.

Certain aspects of the present invention concern the purification, and in particular embodiments, the substantial purification, of an encoded PIMS protein or peptide. The term “purified PIMS protein or peptide” as used herein, is intended to refer to a composition, isolatable from other components, wherein the PIMS protein or peptide is purified to any degree relative to its naturally obtainable state. A purified PIMS protein or peptide therefore also refers to a PIMS protein or peptide, free from the environment in which it may naturally occur.

Generally, “purified” will refer to a PIMS protein composition that has been subjected to fractionation to remove various other components, and which composition substantially retains its expressed biological activity. Where the term “substantially purified” is used, this designation refers to a PIMS protein composition in which the PIMS protein or peptide forms the major component of the composition, such as constituting about 50%, about 60%, about 70%, about 80%, about 90%, about 95%, about 99% or more of the protein, by weight, in the composition.

Various methods for quantifying the degree of purification of the PIMS protein will be known to those of skill in the art. These include, for example, determining the specific binding activity of an active fraction, or assessing the amount of PIMS polypeptides within a fraction by SDS/PAGE analysis. A preferred method for assessing the purity of a PIMS protein fraction is to calculate the binding activity of the fraction, to compare it to the binding activity of the initial extract, and to thus calculate the degree of purification, herein assessed by a “-fold purification number.” The actual units used to represent the amount of binding activity will, of course, be dependent upon the particular assay technique chosen to follow the purification and whether or not the expressed PIMS protein or peptide exhibits a detectable binding activity.

Various techniques suitable for use in PIMS protein purification are well known to those of skill in the art. These include, for example, precipitation with ammonium sulfate, PEG, antibodies and the like, or by heat denaturation, followed by centrifugation; chromatography steps such as ion exchange, gel filtration, reverse phase, hydroxylapatite and affinity chromatography; isoelectric focusing; gel electrophoresis; and combinations of these and other known techniques. As is generally known in the art, it is believed that the order of conducting the various purification steps may be changed, or that certain steps may be omitted, and still result in a suitable method for the preparation of a substantially purified PIMS protein.

There is no general requirement that the PIMS protein always be provided in its most purified state. Indeed, it is contemplated that less substantially purified PIMS proteins will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in greater purification than the same technique utilizing a low pressure chromatography system. Methods exhibiting a lower degree of relative purification may have advantages in total recovery of PIMS protein product, or in maintaining binding activity of an expressed PIMS protein.

It is known that the migration of a polypeptide can vary, sometimes significantly, with different conditions of SDS/PAGE (Capaldi et al., Biochem. Biophys. Res. Comm., 76:425, 1977). It will therefore be appreciated that under differing electrophoresis conditions, the apparent molecular weights of purified or partially purified PIMS protein expression products may vary.

### Effector Cells

Effector cells for inducing, e.g., ADCC against a target cell include human leukocytes, macrophages, monocytes, activated neutrophils, activated natural killer (NK) cells, and eosinophils. Effector cells express FCαR (CD89), FcγRI, FcγRII, FcγRIII, and/or FCεR1 and include, for example, monocytes and activated neutrophils. Expression of FcγRI, e.g., has been found to be up-regulated by interferon gamma (IFN-γ). This enhanced expression increases the cytotoxic activity of monocytes and neutrophils against target cells. Accordingly, effector cells may be activated with (IFN-γ) or other cytokines (e.g., TNF-α or β, colony stimulating factor, IL-2) to increase the presence of FcγRI on the surface of the cells prior to being contacted with a PIMS protein of the invention.

The PIMS proteins of the invention provide an antibody effector function, such as antibody-dependent effector cell-mediated cytotoxicity (ADCC), for use against a target cell. PIMS proteins with effector function are administered alone, as taught herein, or after being coupled to an effector cell, thereby forming an “activated effector cell.”An “activated effector cell” is an effector cell, as defined herein, linked to a PIMS protein, also as defined herein, such that the effector cell is effectively provided with a targeting function prior to administration.

Activated effector cells are administered in vivo as a suspension of cells in a physiologically acceptable solution. The number of cells administered is on the order of 10⁸-10⁹, but will vary depending on the therapeutic purpose. In general, the amount will be sufficient to obtain localization of the effector cell at the target cell, and to provide a desired level of effector cell function in that locale, such as cell killing by ADCC and/or phagocytosis. The term “physiologically acceptable solution,” as used herein, is intended to include any carrier solution which stabilizes the targeted effector cells for administration in vivo including, for example, saline and aqueous buffer solutions, solvents, antibacterial and antifungal agents, isotonic agents, and the like.

Accordingly, another aspect of the invention provides a method of inducing a specific antibody effector function, such as ADCC, against a cell in a subject, comprising administering to the subject the PIMS protein (or encoding nucleic acid) or activated effector cell in a physiologically acceptable medium. Routes of administration can vary and suitable administration routes will be determined by those of skill in the art based on a consideration of case-specific variables and routine procedures, as is known in the art.

### Diseases, Disorders and Conditions

The invention provides PIMS proteins, and variant and derivative thereof, that bind to one or more binding partners and those binding events are useful in the treatment, prevention, or amelioration of a symptom associated with a disease, disorder or pathological condition, preferably one afflicting humans. In preferred embodiments of these methods, the PIMS protein associates a cell bearing a target, such as a tumor-specific cell-surface marker, with an effector cell, such as a cell of the immune system exhibiting cytotoxic activity. In other embodiments, the PIMS protein having more than one specific binding site specifically binds two different disease-, disorder- or condition-specific cell-surface markers to ensure that the correct target is associated with an effector cell, such as a cytotoxic cell of the immune system. Additionally, the PIMS protein can be used to induce or increase antigen activity, or to inhibit antigen activity. PIMS proteins are also suitable for combination therapies and palliative regimes.

In one aspect, the present invention provides compositions and methods useful for treating or preventing diseases and conditions characterized by aberrant levels of antigen activity associated with a cell. These diseases include cancers and other hyperproliferative conditions, such as hyperplasia, psoriasis, contact dermatitis, immunological disorders, and infertility. A wide variety of cancers, including solid tumors and leukemias, are amenable to the compositions and methods disclosed herein. Types of cancer that may be treated include, but are not limited to: adenocarcinoma of the breast, prostate, and colon; all forms of bronchogenic carcinoma of the lung; myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell). Additional types of cancers suitable for treatment include: histiocytic disorders; leukemia; histiocytosis malignant; Hodgkin's disease; immunoproliferative small; non-Hodgkin's lymphoma; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; and trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. The types of cancers that may be treated also include, but are not limited to, angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia. The invention further provides compositions and methods useful in the treatment of other conditions in which cells have become immortalized or hyperproliferative due to abnormally high expression of antigen.

Exemplifying the variety of hyperproliferative disorders amenable to the compositions and methods of the invention are B-cell cancers, including B-cell lymphomas (such as various forms of Hodgkin's disease, non-Hodgkins lymphoma (NHL) or central nervous system lymphomas), leukemias (such as acute lymphoblastic leukemia (ALL), chronic lymphocytic leukemia (CLL), Hairy cell leukemia and chronic myoblastic leukemia), and myelomas (such as multiple myeloma). Additional B cell cancers include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt's lymphoma/leukemia, B-cell proliferations of uncertain malignant potential, lymphomatoid granulomatosis, and post-transplant lymphoproliferative disorder.

Disorders characterized by autoantibody production are often considered autoimmune diseases. Autoimmune diseases amenable to treatment or symptom amelioration with the compositions and methods of the invention include, but are not limited to, arthritis, rheumatoid arthritis, juvenile rheumatoid arthritis, osteoarthritis, polychondritis, psoriatic arthritis, psoriasis, dermatitis, polymyositis/dermatomyositis, inclusion body myositis, inflammatory myositis, toxic epidermal necrolysis, systemic scleroderma and sclerosis, CREST syndrome, responses associated with inflammatory bowel disease, Crohn's disease, ulcerative colitis, respiratory distress syndrome, adult respiratory distress syndrome (ARDS), meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), subacute cutaneous lupus erythematosus, discoid lupus, lupus myelitis, lupus cerebritis, juvenile onset diabetes, multiple sclerosis, allergic encephalomyelitis, neuromyelitis optica, rheumatic fever, Sydenham's chorea, immune responses associated with acute and delayed hypersensitivity mediated by cytokines and T-lymphocytes, tuberculosis, sarcoidosis, granulomatosis including Wegener's granulomatosis and Churg-Strauss disease, agranulocytosis, vasculitis (including hypersensitivity vasculitis/angiitis, ANCA and rheumatoid vasculitis), aplastic anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, central nervous system (CNS) inflammatory disorders, multiple organ injury syndrome, myasthenia gravis, antigen-antibody complex mediated diseases, anti-glomerular basement membrane disease, anti-phospholipid antibody syndrome, allergic neuritis, Behcet disease, Castleman's syndrome, Goodpasture's syndrome, Lambert-Eaton Myasthenic Syndrome, Reynaud's syndrome, Sjogren's syndrome, Stevens-Johnson syndrome, solid organ transplant rejection, graft-versus-host disease (GVHD), bullous pemphigoid, pemphigus, autoimmune polyendocrinopathies, seronegative spondyloarthropathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic thrombocytopenic purpura (TTP), Henoch-Schonlein purpura, autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, lymphoid interstitial pneumonitis (HIV), bronchiolitis obliterans (non-transplant) vs NSIP, Guillain-Barre Syndrome, large-vessel vasculitis (including polymyalgia rheumatica and giant cell (Takayasu's) arteritis), medium vessel vasculitis (including Kawasaki's disease and polyarteritis nodosa), polyarteritis nodosa (PAN) ankylosing spondylitis, Berger's disease (IgA nephropathy), rapidly progressive glomerulonephritis, primary biliary cirrhosis, Celiac sprue (gluten enteropathy), cryoglobulinemia, cryoglobulinemia associated with hepatitis, amyotrophic lateral sclerosis (ALS), coronary artery disease, familial Mediterranean fever, microscopic polyangiitis, Cogan's syndrome, Whiskott-Aldrich syndrome and thromboangiitis obliterans.

Rheumatoid arthritis (RA) is a chronic disease characterized by inflammation of the joints, leading to swelling, pain, and loss of function. Patients having RA for an extended period usually exhibit progressive joint destruction, deformity, disability and even premature death. Beyond RA, inflammatory diseases, disorders and conditions in general are amenable to treatment, prevention or amelioration of symptoms (e.g., heat, pain, swelling, redness) associated with the process of inflammation, and the compositions and methods of the invention are beneficial in treating, preventing or ameliorating aberrant or abnormal inflammatory processes, including RA.

Crohn's disease and a related disease, ulcerative colitis, are the two main disease categories that belong to a group of illnesses called inflammatory bowel disease (IBD). Crohn's disease is a chronic disorder that causes inflammation of the digestive or gastrointestinal (GI) tract. Although it can involve any area of the GI tract from the mouth to the anus, it most commonly affects the small intestine and/or colon. In ulcerative colitis, the GI involvement is limited to the colon. Crohn's disease may be characterized by antibodies against neutrophil antigens, i.e., the “perinuclear anti-neutrophil antibody” (pANCA), and Saccharomyces cerevisiae, i.e. the “anti-Saccharomyces cerevisiae antibody” (ASCA). Many patients with ulcerative colitis have the pANCA antibody in their blood, but not the ASCA antibody, while many Crohn's patients exhibit ASCA antibodies, and not pANCA antibodies. One method of evaluating Crohn's disease is using the Crohn's Disease Activity Index (CDAI), based on 18 predictor variable scores collected by physicians. CDAI values of 150 and below are associated with quiescent disease; values above that indicate active disease, and values above 450 are seen with extremely severe disease [Best et al., “Development of a Crohn's disease activity index.” Gastroenterology 70:439-444 (1976)]. However, since the original study, some researchers use a ‘subjective value’ of 200 to 250 as a healthy score.

Systemic Lupus Erythematosus (SLE) is an autoimmune disease caused by recurrent injuries to blood vessels in multiple organs, including the kidney, skin, and joints. In patients with SLE, a faulty interaction between T cells and B-cells results in the production of autoantibodies that attack the cell nucleus. There is general agreement that autoantibodies are responsible for SLE, so new therapies that deplete the B-cell lineage, allowing the immune system to reset as new B-cells are generated from precursors, would offer hope for long lasting benefit in SLE patients.

Multiple sclerosis (MS) is also an autoimmune disease. It is characterized by inflammation of the central nervous system and destruction of myelin, which insulates nerve cell fibers in the brain, spinal cord, and body. Although the cause of MS is unknown, it is widely believed that autoimmune T cells are primary contributors to the pathogenesis of the disease. However, high levels of antibodies are present in the cerebral spinal fluid of patients with MS, and some theories predict that the B-cell response leading to antibody production is important for development of the disease.

Autoimmune thyroid disease results from the production of autoantibodies that either stimulate the thyroid to cause hyperthyroidism (Graves' disease) or destroy the thyroid to cause hypothyroidism (Hashimoto's thyroiditis). Stimulation of the thyroid is caused by autoantibodies that bind and activate the thyroid stimulating hormone (TSH) receptor. Destruction of the thyroid is caused by autoantibodies that react with other thyroid antigens.

Additional diseases, disorders, and conditions amenable to the benefits provided by the compositions and methods of the invention include the aforementioned Sjogren's syndrome, which is an autoimmune disease characterized by destruction of the body's moisture-producing glands. Further, immune thrombocytopenic purpura (ITP) is caused by autoantibodies that bind to blood platelets and cause their destruction, and this condition is suitable for application of the materials and methods of the invention. Myasthenia Gravis (MG), a chronic autoimmune neuromuscular disorder characterized by autoantibodies that bind to acetylcholine receptors expressed at neuromuscular junctions leading to weakness of the voluntary muscle groups, is a disease having symptoms that are treatable using the composition and methods of the invention, and it is expected that the invention will be beneficial in treating and/or preventing MG. Still further, Rous Sarcoma Virus infections are expected to be amenable to treatment, or amelioration of at least one symptom, with the compositions and methods of the invention.

Another aspect of the present invention is using the materials and methods of the invention to prevent and/or treat any hyperproliferative condition of the skin including psoriasis and contact dermatitis or other hyperproliferative diseases. Psoriasis is characterized by autoimmune inflammation in the skin and is also associated with arthritis in 30% of cases, as well as scleroderma, and inflammatory bowel disease (including Crohn's disease and ulcerative colitis). It has been demonstrated that patients with psoriasis and contact dermatitis have elevated antigen activity within these lesions (Ogoshi et al., J. Inv. Dermatol., 110:818-23 [1998]).

The PIMS proteins can deliver a cytotoxic cell of the immune system, for example, directly to cells within the lesions expressing high levels of antigen. The PIMS proteins can be administered subcutaneously in the vicinity of the lesions, or by using any of the various routes of administration described herein and others which are well known to those of skill in the art.

Also contemplated is the treatment of idiopathic inflammatory myopathy (IIM), including dermatomyositis (DM) and polymyositis (PM). Inflammatory myopathies have been categorized using a number of classification schemes. Miller's classification schema (Miller, Rheum Dis Clin North Am. 20:811-826, 1994) identifies 2 idiopathic inflammatory myopathies (IIM), polymyositis (PM) and dermatomyositis (DM).

Polymyositis and dermatomyositis are chronic, debilitating inflammatory diseases that involve muscle and, in the case of DM, skin. These disorders are rare, with a reported annual incidence of approximately 5 to 10 cases per million adults and 0.6 to 3.2 cases per million children per year in the United States (Targoff, Curr Probl Dermatol. 1991, 3:131-180). Idiopathic inflammatory myopathy is associated with significant morbidity and mortality, with up to half of affected adults noted to have suffered significant impairment (Gottdiener et al., Am J Cardiol. 1978, 41:1141-49). Miller (Rheum Dis Clin North Am. 1994, 20:811-826 and Arthritis and Allied Conditions, Ch. 75, Eds. Koopman and Moreland, Lippincott Williams and Wilkins, 2005) sets out five groups of criteria used to diagnose IIM, i.e., Idiopathic Inflammatory Myopathy Criteria (IIMC) assessment, including muscle weakness, muscle biopsy evidence of degeneration, elevation of serum levels of muscle-associated enzymes, electromagnetic triad of myopathy, evidence of rashes in dermatomyositis, and also evidence of autoantibodies as a secondary criterion.

IIM-associated factors, including muscle-associated enzymes and autoantibodies include, but are not limited to, creatine kinase (CK), lactate dehydrogenase, aldolase, C-reactive protein, aspartate aminotransferase (AST), alanine aminotransferase (ALT), as well as antinuclear autoantibody (ANA), myositis-specific antibodies (MSA), and antibody to extractable nuclear antigens.

Preferred autoimmune diseases amenable to the methods of the invention include Crohn's disease, Guillain-Barre syndrome (GBS; also known as acute inflammatory demyelinating polyneuropathy, acute idiopathic polyradiculoneuritis, acute idiopathic polyneuritis and Landry's ascending paralysis), lupus erythematosus, multiple sclerosis, myasthenia gravis, optic neuritis, psoriasis, rheumatoid arthritis, hyperthyroidism (e.g., Graves' disease), hypothyroidism (e.g., Hashimoto's disease), Ord's thyroiditis (a thyroiditis similar to Hashimoto's disease), diabetes mellitus (type 1), aplastic anemia, Reiter's syndrome, autoimmune hepatitis, primary biliary cirrhosis, antiphospholipid antibody syndrome (APS), opsoclonus myoclonus syndrome (OMS), temporal arteritis (also known as “lgiant cell arteritis”), acute disseminated encephalomyelitis (ADEM), Goodpasture's syndrome, Wegener's granulomatosis, coeliac disease, pemphigus, canine polyarthritis, and warm autoimmune hemolytic anemia. In addition, the invention contemplates methods for the treatment, or amelioration of a symptom associated with, endometriosis, interstitial cystitis, neuromyotonia, scleroderma, vitiligo, vulvodynia, Chagas' disease leading to Chagasic cardiopathy (cardiomegaly), sarcoidosis, chronic fatigue syndrome, and dysautonomia

The complement system is believed to play a role in many diseases with an immune component, such as Alzheimer's disease, asthma, lupus erythematosus, various forms of arthritis, autoimmune heart disease and multiple sclerosis, all of which are contemplated as diseases, disorders or conditions amenable to treatment or symptom amelioration using the methods according to the invention.

Certain constant sub-regions are preferred, depending on the particular effector function or functions to be exhibited by a multispecific single-chain binding molecule. For example, IgG (IgG1, 2, or 3) and IgM are preferred for complement activation, IgG of any subtype is preferred for opsonization and toxin neutralization; IgA is preferred for pathogen binding; and IgE for binding of such parasites as worms.

By way of example, FcRs recognizing the constant region of IgG antibodies have been found on human leukocytes as three distinct types of Fcγ receptors, which are distinguishable by structural and functional properties, as well as by antigenic structures detected by anti-CD monoclonal antibodies. They are known as FcγRI, FcγRII, and FcγRIII, and are differentially expressed on (overlapping) subsets of leukocytes.

FcγRI (CD64), a high-affinity receptor expressed on monocytes, macrophages, neutrophils, myeloid precursors and dendritic cells, comprises isoforms 1a and 1b. FcγRI has a high affinity for monomeric human IgG1 and IgG3. Its affinity for IgG4 is about 10 times lower, while it does not bind IgG2. FcγRI does not show genetic polymorphism.

FcγRII (CD32), comprised of isoforms lla, llb1, llb2, llb3 and llc, is the most widely distributed human FcγR type, being expressed on most types of blood leukocytes, as well as on Langerhans cells, dendritic cells and platelets. FcγRII is a low-affinity receptor that only binds aggregated IgG. It is the only FcγR class able to bind IgG2. FcγRIIa shows genetic polymorphism, resulting in two distinct allotypes, FcγRIIa-H131 and FcγRIIa-R131. This functional polymorphism is attributable to a single amino acid difference: a histidine (H) or an arginine (R) residue at position 131, respectively, which is critical for IgG binding. FcγRIIa readily binds human IgG subisotypes other than IgG4. The FcγRlla-H131 has a much higher affinity for complexed IgG2 than the FcγRlla-R131 allotype.

FcγRIII (CD16) has two isoforms, both of which are able to bind IgG1 and IgG3. The FcγRIIIa, with an intermediate affinity for IgG, is expressed on macrophages, monocytes, natural killer (NK) cells, and subsets of T cells. FcγRIIIb is a low-affinity receptor for IgG, selectively expressed on neutrophils. It is a highly mobile receptor with efficient collaboration with other membrane receptors. Studies with myeloma IgG dimers have shown that only IgG1 and IgG3 bind to FcγRIIIb (with low affinity), while no binding of IgG2 and IgG4 has been found. The FcγRIIIb bears a co-dominant, bi-allelic polymorphism, the allotypes being designated NA1 (Neutrophil Antigen) and NA2.

Yet another aspect of the invention is use of the materials and methods of the invention to combat, by treating, preventing or mitigating the effects of, infection, resulting from any of a wide variety of infectious agents. The PIMS molecules of the invention are designed to efficiently and effectively recruit the host organism's immune system to resist infection arising from a foreign organism, a foreign cell, a foreign virus or a foreign inanimate object.

Infectious cells contemplated by the invention include any known infectious cell including, but not limited to, any of a variety of bacteria (e.g., pathogenic E. coli, S. typhimurium, P. aeruginosa, B. anthracis, C. botulinum, C. difficile, C. perfringens, H. pylori, V. cholerae, and the like), mycobacteria, mycoplasma, fungi (including yeast and molds), and parasites (including any known parasitic member of the Protozoa, Trematoda, Cestoda and Nematoda). Infectious viruses include, but are not limited to, eukaryotic viruses (e.g., adenovirus, bunyavirus, herpesvirus, papovavirus, paramyxovirus, picornavirus, poxvirus, reovirus, retroviruses, and the like) as well as bacteriophage. Foreign objects include objects entering an organism, preferably a human, regardless of mode of entry and regardless of whether harm is intended. In view of the increasing prevalence of multi-drug-resistant infectious agents (e.g., bacteria), particularly as the causative agents of nosocomial infection, the materials and methods of the invention, providing an approach to treatment that avoids the difficulties imposed by increasing antibiotic resistance, are expected to provide a welcome addition to the medical and veterinary arsenals available to combat these conditions.

Diseases, conditions or disorders associated with infectious agents and amenable to treatment (prophylactic or therapeutic) with the materials and methods disclosed herein include, but are not limited to, anthrax, aspergillosis, bacterial meningitis, bacterial pneumoniae (e.g., chlamydia pneumoniae), blastomycosis, botulism, brucellosis, candidiasis, cholera, ciccidioidomycosis, cryptococcosis, diahhreagenic, enterohemorrhagic or enterotoxigenic E. coli, diphtheria, glanders, histoplasmosis, legionellosis, leprosy, listeriosis, nocardiosis, pertussis, salmonellosis, scarlet fever, sporotrichosis, strep throat, toxic shock syndrome, traveler's diarrhea, and typhoid fever.

Additional aspects and details of the invention will be apparent from the following examples, which are intended to be illustrative rather than limiting.

### EXAMPLE 1

### PIMS Construction

As illustrated in FIG. 1, a PIMS peptide has the following structural domains: an N-terminally disposed constant sub-region and at least one C-terminally disposed specific binding site, with each of these two domains linked by a PIMS linker that may be derived from an immunoglobulin hinge. In some embodiments, a hinge region derived from an immunoglobulin is located N-terminal to the constant sub-region, although the constant sub-region is still disposed N-terminally to the specific binding site(s). In some embodiments, the N-terminus of the nascently expressed PIMS molecule may be a leader peptide, as would be known in the art to be useful in expression and secretion of an encoded peptide. This leader peptide, moreover, may be covalently joined to a region derived from an immunoglobulin hinge or directly to a constant sub-region (PIMS molecules lacking an N-terminal domain derived from a hinge region).

Recombinant engineering of vehicles for PIMS-encoding polynucleotides is expected to facilitate construction of these various PIMS molecules, for example by directed placement of suitable restriction endonuclease cleavage sites. In addition to generating a repertoire of PIMS polynucleotides and encoded PIMS peptides by recombinant engineering, it is contemplated that various mutagenic techniques, including site-directed mutagenesis, will be used to produce a variety of PIMS and variants thereof. For example, site-directed mutagenesis is suitable for altering the codons specifying cysteine residues capable of participating in inter-chain disulfide bond formation. Typically, such Cys residues would be located in the linker region joining the constant sub-region and at least one specific binding site, and/or in an N-terminal hinge region. Exemplary hinges include regions derived from IgG1 hinges, wherein the derived hinge region has a single Cys residue or has two Cys residues.

Also apparent from FIG. 1 is the relative placement of binding domains participating in a given binding site. For example, the invention comprehends PIMS molecules having two binding domains of a given binding site in either orientation. For example, one PIMS molecule has the orientation N-[optional leader peptide]-[optional hinge region]-constant sub-region-PIMS linker-V_{L}-V_{H}-C and another PIMS molecule has the orientation N-[optional leader peptide]-[optional hinge region]-constant sub-region-PIMS linker-V_{H}-V_{L}-C. Other exemplary PIMS are molecules wherein the C-terminal binding domain is composed of a single variable domain, either V_{H} or V_{L} as well as two variable domains, V_{H}-V_{H} or V_{L}-V_{L} that might have identical, similar or diverse binding properties, such as one V_{H} that binds the CD3 extracellular domain or two V_{H} domains that bind identical or separate epitopes on the CD3 extracellular domain or two V_{H} domains in which one domain binds the CD3 extracellular domain and the other binds, for example, the extracellular domain of CD28. PIMS molecules include polypeptides (and encoding polynucleotides) that contain a constant sub-region derived from an immunoglobulin, a PIMS linker that may be derived from an immunoglobulin, and at least one specific binding domain, wherein the constant sub-region is disposed N-terminal relative to each of the specific binding domains of the molecule.

PIMS molecules were constructed to take advantage of a pre-existing expression vector cassette strategy that allows swapping of various components contained in a SMIP, Scorpion or PIMS. The strategy and molecular structures are described in U.S. Patent Application No. 60/813,261, particularly in Example 3 therein, and U.S. Ser. No. 60/813,261 is incorporated herein by reference. Using a SMIP cassette, the scFv comprising “Binding Domain 1” (BD1) and the first 400 nucleotides of the Effector Domain, in this case the hinge-C_{H2}-C_{H3} of human IgG1, were removed by completely digesting the vector cassette DNA with the restriction enzymes Agel and BsrGI (New England Biolabs). The largest of the resultant DNA fragments, containing the entire pD18 vector, the human VK3 leader and the C-terminal 300 nucleotides of the human IgG1, was gel-purified and stored at −20° C. for future use.

To generate PIMS W0001 through PIMS W0007, oligonucleotide primers (W0001F-WO007F) were designed such that they encoded the last 2 amino acids in the VK3 leader, which correspond to the Agel cleavage site (ACCGGT encoding Thr-Gly), a series of intervening amino acids to assure that signal peptide cleavage would not occur within the hinge region of the IgG1 Effector Domain, then the nucleotides containing the recognition sequence for the restriction enzyme XhoI in the correct reading frame. This ensured that the open reading frame was maintained from the leader sequence, through the spacer amino acids and continuing through the entire Effector Domain. These primers were used in a PCR amplification reaction along with a reverse primer, IgBsrG1R, to produce a PCR product that was then digested to completion with both AgeI and BsrGI, gel-purified, and ligated to the previously digested vector described above.

This design resulted in 7 PIMS molecules that differed only in the amino acids encoded between the leader sequence and the beginning of the Effector Domain (see the amino acid sequences of PIMS W0001-W0007 in the sequence listing). All other sequences in these molecules are identical. These “spacer” amino acids serve not only to limit the leader peptide cleavage during translation, but also as a template from which to guide protein engineering efforts to affect Effector Domain function, protein expression in various biological systems, as well as a point of insertion for other biologically relevant peptides.

In greater detail, an anti-CD28 PIMS molecule was constructed by initially diluting an aliquot of Scorpion molecule S0033 to a concentration of 5 μg/mL. One μL was used as the template in a PCR containing 20 μmol each from 100 μM stock solutions of primers W0001F and 12HL-XbaR (oligonucleotides used, e.g., as primers, are provided in Table 6) in a total reaction volume of 50 μL in Platinum PCR Supermix High Fidelity PCR mix (Invitrogen). This PCR mixture was then placed in an ABI 9700 Thermal cycler and after an initial 3-minute incubation at 95° C., was cycled 30 times at 94° C. for 30 seconds, 60° C. for 15 seconds and 68° C. for 2 minutes, followed by a final 3-minute extension at 68° C. The reactions were then brought to room temperature, and purified over Qiagen MinElute columns according to the manufacturer's protocol to remove salts, excess primers and enzymes. This purified PCR product was then eluted from the columns in a total volume of 20 μL in 10 mM Tris, pH 8. 4 μL of the PCR product was then mixed with 1 μL of 1 M sodium chloride solution and carefully mixed while adding 1 μL of pCR2.1-TOPO vector mix (Invitrogen) and the reaction mix was incubated on the benchtop for 20 minutes. 2 μL of this reaction was then mixed with 20 μL of chemically competent bacterial strain TOP 10 and incubated on ice 15 minutes, heat-shocked at 42° C. for 30 seconds, brought to 200 μL in SOC broth, and incubated at 37° C. for 30 minutes, all per manufacturer's instructions (Invitrogen). The bacteria were then plated on LB agar+50 μg/mL Kanamycin+X-gal/IPTG Plates (Teknova). The plates were incubated overnight at 37° C. and the next day colonies were inoculated into a deep-well, 96-well plate with 1 mL T-broth+Kanamycin 50 μg/mL (Teknova) per well and shaken overnight at 37° C. The following day, 20 μL was removed from each well and mixed with 20 μL of a 50% glycerol solution in a 96-well plate, which was then stored overnight at −20° C. The remaining bacteria in the deep-well plate were then pelleted at 4K rpm in a Beckman Avanti centrifuge for 10 minutes and the cleared broth was removed, leaving only the bacterial pellets. The plate was then placed on a QiaRobot 8000 and plasmid DNA was purified using the QiaPrep Turbo Kit provided by the manufacturer for use on the QiaRobot 8000. Purified DNA was prepared in this manner for all colonies picked for analysis.

For PCR sequencing reactions, 5 μL of the purified DNA was taken from each well of the 96-well plate and pipetted into 2 duplicate 96-well plates. A mixture of 4 pmoles DNA sequencing primer M13R (plate 1) or M13F (plate 2) and 4 μL BigDye Terminator Sequencing Mix version 3.1 (ABI) was added to each well for a total volume of 10 μL. These PCR sequencing reactions were then placed in an ABI 9700 Thermal cycler and cycled 25 times at 96° C., 10 seconds, 50° C., 5 seconds and 60° C., 6 minutes. Afterwards, the sequencing reactions were diluted to 20 μL in sterile water and loaded onto pre-spun Centri-Sep G-25 columns (Princeton Separations) to remove unincorporated labeled nucleotides from the reactions. The resultant PCR products were then loaded and run on an ABI 3130-XL DNA Sequencer and the DNA sequences analyzed using the ConTig Express Module of Vector NTI 10.0 (Invitrogen). DNA from one clone containing the desired DNA sequence was then digested with the restriction enzymes Agel and XbaI (both from New England BioLabs) in a 60 μL reaction. Digests were incubated at 37° C. for 6 hours, then loaded onto a 1% agarose TAE gel and run at 110V for 40 minutes. At this point, the 1.5 Kbp W0001 DNA (SEQ ID NO:358) band could be easily resolved from the 4 Kbp vector DNA band. The W0001 DNA was excised from the agarose gel and purified on a Qiagen MinElute column using the manufacturer's instructions for DNA extraction form agarose gels (Qiagen). The resulting digested and purified DNA was eluted in a volume of 10 μL. Two uL of this DNA was used in a ligation reaction including 10 ng pD18+VK3 leader DNA digested with AgeI and XbaI, 1.5 μL of 10× Ligation buffer (Roche) and 1 μL T4 DNA ligase (Roche) in a 15 μL reaction that was incubated overnight at room temperature. After ligation, 5 μL was transformed into chemically competent TOP 10, as described above, with the exception that the cells were plated on 2xYT+carbenicillin (100 μg/mL) plates and incubated overnight at 37° C. Colonies were grown as described above, screened for the presence of a 1.5 Kbp AgeI-XbaI DNA fragment, and again sequenced to confirm that the DNA had the desired nucleotide sequence. A single clone, now identified as W0001, was amplified in a 100 mL T-broth+carbenicillin overnight culture. DNA was prepared from this bacterial culture using a Qiagen Maxi Prep Kit according to the manufacturer's protocol. The resultant DNA preparation was quantified by absorbance at 260 nm using a Nanodrop spectrophotometer.

TABLE 6

| | Sequence |
|---|---|
| Name | Identifier |
| Heavy Chain GSP1 primer | 251 |
| Nested heavy chain GSP2 primer | 252 |
| Light chain GSP1 primer | 253 |
| Nested light chain GSP2 primer | 254 |
| 5′ RACE abridged anchor primer | 255 |
| T7 sequencing primer | 256 |
| M13 reverse primer | 257 |
| PCR primer hVK3L-F3H3 | 258 |
| PCR primer hVK3L-F2 | 259 |
| PCR primer hVK3L-F1-2H7VL | 260 |
| PCR primer2H7VH-NheF | 261 |
| PCR primer G4S-NheR | 262 |
| PCR primer 015VH-XhoR | 263 |
| PCR primer G1H-S-XHO | 265 |
| PCR primer CH3R-EcoR1 | 266 |
| PCR primer G1-XBA-R | 267 |
| PCR primer G4SLinkR1-S | 268 |
| PCR primer G4SLinkR1-AS | 269 |
| PCR primer 2E12VLXbaR | 270 |
| PCR primer 2E12VLR1F | 271 |
| PCR primer 2E12VHR1F | 272 |
| PCR primer 2E12VHXbaR | 273 |
| PCR primer 2e12VHdXbaF1 | 274 |
| PCR primer 2e12VHdXbaR1 | 275 |
| PCR primer IgBsrG1F | 276 |
| PCR primer IgBsrG1R | 277 |
| PCR primer M13R | 278 |
| PCR primer M13F | 279 |
| PCR primer T7 | 280 |
| PCR primer pD18F-17 | 281 |
| PCR primer pD18F-20 | 282 |
| PCR primer pD18F-1 | 283 |
| PCR primer pD18R-s | 284 |
| PCR primer CH3seqF1 | 285 |
| PCR primer CH3seqF2 | 286 |
| PCR primer CH3seqR1 | 287 |
| PCR primer CH3seqR2 | 288 |
| PCR primer L1-11R | 289 |
| PCR primer L1-6R | 290 |
| PCR primer L3R | 291 |
| PCR primer L4R | 292 |
| PCR primer L5R | 293 |
| PCR primer IgBsrG1F | 294 |
| PCR primer L-CPPCPR | 295 |
| CD37 binding domain primer G281LH-NheR | 309 |
| CD37 binding domain primer G281LH-NheF | 310 |
| CD37 binding domain primer G281-LH-LPinF | 311 |
| CD37 binding domain primer G281-LH-HXhoR | 312 |
| CD37 binding domain primer G281-LH-LEcoF | 313 |
| CD37 binding domain primer G281-LH-HXbaR | 314 |
| CD37 binding domain primer G281-HL-HF | 315 |
| CD37 binding domain primer G281-HL-HR3 | 316 |
| CD37 binding domain primer G281-HL-HR2 | 317 |
| CD37 binding domain primer G281-HL-HNheR | 318 |
| CD37 binding domain primer G281-HL-LNheF | 319 |
| CD37 binding domain primer G281-HL-LXhoR | 320 |
| CD37 binding domain primer G281-HL-LXbaR | 321 |
| CD37 binding domain primer G281-HL-EcoF | 322 |
| CD3 binding domain primer (G19-4 temp.) 194-LH-LF1 | 323 |
| CD3 binding domain primer (G19-4 temp.) 194-LF2 | 324 |
| CD3 binding domain primer (G19-4 temp.) 194-LF3 | 325 |
| CD3 binding domain primer (G19-4 temp.) 194-LF4 | 326 |
| CD3 binding domain primer (G19-4 temp.) 194-LF5 | 327 |
| CD3 binding domain primer (G19-4 temp.) 194-LF6 | 328 |
| CD3 binding domain primer (G19-4 temp.) 194-LF7 | 329 |
| CD3 binding domain primer (G19-4 temp.) 194-LR7 | 330 |
| CD3 binding domain primer (G19-4 temp.) 194-LR6 | 331 |
| CD3 binding domain primer (G19-4 temp.) 194-LR5 | 332 |
| CD3 binding domain primer (G19-4 temp.) 194-LR4 | 333 |
| CD3 binding domain primer (G19-4 temp.) 194-LR3 | 334 |
| CD3 binding domain primer (G19-4 temp.) 194-LR2 | 335 |
| CD3 binding domain primer (G19-4 temp.) 194-LH-LR1 | 336 |
| CD3 binding domain primer (G19-4 temp.) 194-LH-HF1 | 337 |
| CD3 binding domain primer (G19-4 temp.) 194-HF2 | 338 |
| CD3 binding domain primer (G19-4 temp.) 194-HF3 | 339 |
| CD3 binding domain primer (G19-4 temp.) 194-HF4 | 340 |
| CD3 binding domain primer (G19-4 temp.) 194-HF5 | 341 |
| CD3 binding domain primer (G19-4 temp.) 194-HF6 | 342 |
| CD3 binding domain primer (G19-4 temp.) 194-HR6 | 343 |
| CD3 binding domain primer (G19-4 temp.) 194-HR5 | 344 |
| CD3 binding domain primer (G19-4 temp.) 194-HR4 | 345 |
| CD3 binding domain primer (G19-4 temp.) 194-HR3 | 346 |
| CD3 binding domain primer (G19-4 temp.) 194-HR2 | 347 |
| CD3 binding domain primer (G19-4 temp.) 194-LH-HR1 | 348 |
| CD3 binding domain primer (G19-4 temp.) 194-HL-HF1 | 349 |
| CD3 binding domain primer (G19-4 temp.) 194-HL-HR1 | 350 |
| CD3 binding domain primer (G19-4 temp.) 194-HL-HR0 | 351 |
| CD3 binding domain primer (G19-4 temp.) 194-HL-LF1 | 352 |
| CD3 binding domain primer (G19-4 temp.) 194-HL-LR3Xho | 353 |
| CD3 binding domain primer (G19-4 temp.) 194-HL-LR3Xba | 354 |
| CD3 binding domain primer (G19-4 temp.) 194-HL-HF1R1 | 355 |
| CD3 binding domain primer (G19-4 temp.) 194-LH-LF1R1 | 356 |
| CD3 binding domain primer (G19-4 temp.) 194-LH-HR1Xba | 357 |
| W0001F PCR primer | 296 |
| W0002F PCR primer | 297 |
| W0003F PCR primer | 298 |
| W0004F PCR primer | 299 |
| W0005F PCR primer | 300 |
| W0006F PCR primer | 301 |
| W0007F PCR primer | 302 |
| W0008F PCR primer | 303 |
| W0009F PCR primer | 304 |
| W0010F PCR primer | 305 |
| W0011F PCR primer | 306 |
| W0012F PCR primer | 307 |
| 12HL-XbaR PCR primer | 308 |

Additional PIMS molecules have been constructed that conform to the general organization of domains found in the W0001 PIMS, i.e., an N-terminal hinge region joined to a constant subregion Fc effector domain comprising a C_{H2} region and a C_{H3} region, followed by a PIMS linker and a C-terminally disposed binding domain. These PIMS molecules have been designated W0002 to W0009 and the sequences are presented in SEQ ID NOS:360-375. W0002-WO007 each comprises the 2E12 binding domain and specifically binds CD28; W0008 comprises the 2Lm20-4 VHVL 12 binding domain and W0009 comprises the 2Lm20-4 V_{H}V_{L} 17, with each specifically binding CD20. Features of the amino acid sequences of these PIMS, as well as exemplary encoding nucleic acid sequences, are presented in Table 7. Thus, it is apparent that a variety of specific binding domains may be used in PIMS to target the molecule as desired.

TABLE 7

| PIMS Molecule | Features |
|---|---|
| W0001 nucleic acid | Human VK3 leader: nucleotides 1-60 |
| (SEQ ID NO: 358) | Leader-hinge junction: 61-69 |
| | Hinge region: 70-114 |
| | CH2CH3 region: 115-765 |
| | Linker peptide: 766-789 |
| | Binding domain: 790-1551 |
| | Binding region linker: 1153-1212 |
| W0001 polypeptide | Human VK3 leader: amino acids 1-20 |
| (SEQ ID NO: 359) | Leader-hinge junction: 21-23 |
| | Hinge region: 24-38 |
| | CH2CH3 region: 39-255 |
| | Linker peptide: 256-263 |
| | Binding domain: 264-516 |
| | Binding region linker: 385-404 |
| W0002 nucleic acid | Human VK3 leader: nucleotides 1-60 |
| (SEQ ID NO: 360) | Leader-hinge junction: 61-69 |
| | Hinge region: 70-114 |
| | CH2CH3 region: 115-765 |
| | Linker peptide: 766-789 |
| | Binding domain: 790-1551 |
| | Binding region linker: 1153-1212 |
| W0002 polypeptide | Human VK3 leader: amino acids 1-20 |
| (SEQ ID NO: 361) | Leader-hinge junction: 21-23 |
| | Hinge region: 24-38 |
| | CH2CH3 region: 39-255 |
| | Linker peptide: 256-262 |
| | Binding domain: 263-516 |
| | Binding region linker: 385-404 |
| W0003 nucleic acid | Human VK3 leader: nucleotides 1-60 |
| (SEQ ID NO: 362) | Leader-hinge junction: 61-69 |
| | Hinge region: 70-114 |
| | CH2CH3 region: 115-765 |
| | Linker peptide: 766-789 |
| | Binding domain: 790-1551 |
| | Binding region linker: 1153-1212 |
| W0003 polypeptide | Human VK3 leader: amino acids 1-20 |
| (SEQ ID NO: 363) | Leader-hinge junction: 21-23 |
| | Hinge region: 24-38 |
| | CH2CH3 region: 39-255 |
| | Linker peptide: 256-263 |
| | Binding domain: 264-516 |
| | Binding region linker: 385-404 |
| W0004 nucleic acid | Human VK3 leader:: nucleotides 1-60 |
| (SEQ ID NO: 364) | Leader-hinge junction: 61-69 |
| | Hinge region: 70-114 |
| | CH2CH3 region: 115-765 |
| | Linker peptide: 766-789 |
| | Binding domain: 790-1551 |
| | Binding region linker: 1153-1212 |
| W0004 polypeptide | Human VK3 leader: amino acids 1-20 |
| (SEQ ID NO: 365) | Leader-hinge junction: 21-23 |
| | Hinge region: 24-38 |
| | CH2CH3 region: 39-255 |
| | Linker peptide: 256-263 |
| | Binding domain: 264-516 |
| | Binding region linker: 385-404 |
| W0005 nucleic acid | Human VK3 leader:: nucleotides 1-60 |
| (SEQ ID NO: 366) | Leader-hinge junction: 61-69 |
| | Hinge region: 70-114 |
| | CH2CH3 region: 115-765 |
| | Linker peptide: 766-789 |
| | Binding domain: 790-1551 |
| | Binding region linker: 1153-1212 |
| W0005 polypeptide | Human VK3 leader: amino acids 1-20 |
| (SEQ ID NO: 367) | Leader-hinge junction: 21-23 |
| | Hinge region: 24-38 |
| | CH2CH3 region: 39-255 |
| | Linker peptide: 256-263 |
| | Binding domain: 264-516 |
| | Binding region linker: 385-404 |
| W0006 nucleic acid | Human VK3 leader:: nucleotides 1-60 |
| (SEQ ID NO: 368) | Leader-hinge junction: 61-69 |
| | Hinge region: 70-114 |
| | CH2CH3 region: 115-765 |
| | Linker peptide: 766-789 |
| | Binding domain: 790-1551 |
| | Binding region linker: 1153-1212 |
| W0006 polypeptide | Human VK3 leader: amino acids 1-20 |
| (SEQ ID NO: 369) | Leader-hinge junction: 21-23 |
| | Hinge region: 24-38 |
| | CH2CH3 region: 39-255 |
| | Linker peptide: 256-263 |
| | Binding domain: 264-516 |
| | Binding region linker: 385-404 |
| W0007 nucleic acid | Human VK3 leader: nucleotides 1-60 |
| (SEQ ID NO: 370) | Leader-hinge junction: 61-66 |
| | Hinge region: 67-111 |
| | CH2CH3 region: 112-762 |
| | Linker peptide: 763-786 |
| | Binding domain: 787-1548 |
| | Binding region linker: 1150-1209 |
| W0007 polypeptide | Human VK3 leader: amino acids 1-20 |
| (SEQ ID NO: 371) | Leader-hinge junction: 21-22 |
| | Hinge region: 23-37 |
| | CH2CH3 region: 38-254 |
| | Linker peptide: 255-262 |
| | Binding domain: 263-515 |
| | Binding region linker: 384-403 |
| W0008 nucleic acid | Human VK3 leader: nucleotides 1-60 |
| (SEQ ID NO: 372) | Leader-hinge junction: 61-66 |
| | Hinge region: 67-111 |
| | CH2CH3 region: 112-762 |
| | Linker peptide: 763-786 |
| | Binding domain: 787-1503 |
| | Binding region linker: 1150-1185 |
| W0008 polypeptide | Human VK3 leader: amino acids 1-20 |
| (SEQ ID NO: 373) | Leader-hinge junction: 21-22 |
| | Hinge region: 23-37 |
| | CH2CH3 region: 38-254 |
| | Linker peptide: 255-262 |
| | Binding domain: 263-501 |
| | Binding region linker: 384-395 |
| W0009 nucleic acid | Human VK3 leader: nucleotides 1-60 |
| (SEQ ID NO: 374) | Leader-hinge junction: 61-66 |
| | Hinge region: 67-111 |
| | CH2CH3 region: 112-762 |
| | Linker peptide: 763-786 |
| | Binding domain: 787-1518 |
| | Binding region linker: 1150-1200 |
| W0009 polypeptide | Human VK3 leader: amino acids 1-20 |
| (SEQ ID NO: 375) | Leader-hinge junction: 21-22 |
| | Hinge region: 23-37 |
| | CH2CH3 region: 38-254 |
| | Linker peptide: 255-262 |
| | Binding domain: 263-506 |
| | Binding region linker: 384-400 |

Still other PIMS molecules have been constructed that explore different binding domains, PIMS linker lengths, PIMS linker sources, and the like. The structures of these additional PIMS molecules, including the features identified for some PIMS molecules in Table 7, are provided in the sequence listing. Some of these PIMS molecules exhibit the organization illustrated in Table 8.

TABLE 8

| Name | Binding Domain | PIMS linker |
|---|---|---|
| W0035 | DR | H7 |
| W0036 | DR | H62 |
| W0056 | DR | H65 |
| W0087 | DR | H64 |

The anti-DR Vk (VL) and Vh (VH) binding regions were constructed by overlapping oligonucleotide PCR. Briefly, 10 pmol of each oligonucleotide (1 ul of 10 uM stock) was added to a PCR reaction and the volume was brought up to 50 ul with PCR SuperMix High Fidelity (Invitrogen cat #10790-020). PCR reactions were conducted according to the following protocol: 94° C. for 2 minutes, then 30 cycles of 94° C. for 30 seconds followed by 50° C. for 20 seconds followed by 68° C. for 3 minutes, and, upon completion of the thirtieth cycle, an incubation at 68° C. for 5 minutes followed by incubation at 4° C. PCR products were cloned into pCR 4-TOPO (Invitrogen cat #45-0030) and sequence verified. Anti-DR Vk (AgeI/BamHI) and anti-DR Vh (BamHI/BclI) fragments were ligated into pD18 scc-p AgeI/BclI digested vector to make the pD18 anti-DR SMIP construct (M0019).

PIMS were made by adding an EcoRI site to the 5′ end and an XbaI site to the 3′ end during PCR amplification. PCR-amplified fragments with restriction sites useful for cloning at each terminus were cloned into the W0011 construct that had been deleted for the TRU-015 (CD20) binding domain by EcoRI/XbaI digestion. More particularly, W0011 EcoRI/XbaI was used for constructing a PIMS with an H7 PIMS linker (W0035), W0011 H62 EcoRI/XbaI was used for constructing a PIMS with an H62 PIMS linker (WO036), and W0011 H64 was used for constructing a PIMS with an H64 as a PIMS linker (WO087). W0056 was made by using the W0036 DNA construct as a template for oligonucleotide-directed mutagenesis with pD18For, a sequencing primer, as the 5′ oligonucleotide and the following 3′ oligonucleotide: 5′-ttcagaattcggagaatgacgtgctttctg-3′ (SEQ ID NO:549). Subsequently, the fragment was cloned back into W0036 using HindIII/BsrGI restriction sites.

### EXAMPLE 2

### Transfection of PIMS-Encoding Polynucleotide into CHO-S Cells

One day prior to performing the transfection experiment proper, each of two sterile flasks were seeded with 5×10⁵ cell/ml in 250 ml of Freestyle™ CHO Expression Medium with 8 mM L-glutamine added. The flasks were incubated at 37° C. with 8% CO₂ and rotated at 70 rpm. On the day of transfection, cells in each flask were counted and Freestyle™ medium was added to provide 10⁶ cells/ml. In separate 15 ml sterile tubes, 313 μg Freestyle™ Max Transfection reagent (1.0 μg/ml) was added to 4,687 μl OptiPro™ SFM and 313 μg W0001 DNA plasmid (1.0 μg/ml) was added to 4,687 μl OptiPro SFM™. The diluted Freestyle™ Max Transfection reagent was added to the diluted W0001 plasmid and incubated at room temperature for 10 minutes. The DNA-Freestyle™ Max Reagent complex was then slowly added to the flask containing the cells and the cells were incubated at 37° C., 8% CO₂ on an orbital shaker rotating at 70 rpm. After seven days, the supernatant from each flask was recovered and recombined to a total volume of 500 ml, which was then filtered through a 2 μm filter. The W0001 protein concentration was 7.38 μg/ml as determined by ELISA.

### EXAMPLE 3

### Expression Studies

Expression studies were performed on the nucleic acids described above that encode specific binding proteins with effector function, or PIMS molecules. Nucleic acids encoding PIMS proteins were transiently transfected into COS cells and the transfected cells were maintained under well known conditions permissive for heterologous gene expression in these cells. DNA was transiently transfected into COS cells using PEI or DEAE-Dextran as previously described (for PEI, see Boussif O. et al., Proc. Natl. Acad. Sci. (USA) 92: 7297-7301, (1995), incorporated herein by reference; Pollard H. et al., J. Biol. Chem. 273:7507-7511, (1998), incorporated herein by reference). Multiple independent transfections of each new molecule were performed in order to determine the average expression level for each new form. For transfection by PEI, COS cells were plated onto 60 mm tissue culture plates in DMEM/10% FBS medium and incubated overnight so that they would be approximately 90% confluent on the day of transfection. Medium was changed to serum-free DMEM containing no antibiotics and incubated for 4 hours. Transfection medium (4 ml/plate) contained serum-free DMEM with 50 μg PEI and 10-20 μg DNA plasmid of interest, such as W0001 plasmid. Transfection medium was mixed by vortexing, incubated at room temperature for 15 minutes, and added to plates after aspirating the existing medium. Cultures were incubated for 3-7 days prior to collection of supernatants. Culture supernatants were assayed for protein expression by SDS-PAGE and Western blotting.

For SDS-PAGE, samples were prepared either from crude culture supernatants (usually 30 μl/well) or purified protein aliquots, containing 8 μg protein per well, with 2×Tris-Glycine SDS Buffer (Invitrogen) being added to a 1× final concentration. Ten (10) μl SeeBlue Marker (Invitrogen, Carlsbad, Calif.) were run to provide MW size standards. The PIMS proteins were subjected to SDS-PAGE analysis on 4-20% Novex Tris-glycine gels (Invitrogen, San Diego, Calif.). Samples were loaded using Novex Tris-glycine SDS sample buffer (2×) under reducing or non-reducing conditions after heating at 95° C. for 3 minutes, followed by electrophoresis at 175V for 60 minutes. Electrophoresis was performed using 1× Novex Tris-Glycine SDS Running Buffer (Invitrogen).

The results of expression studies in COS cells showed that PIMS were expressed at levels intermediate between scorpion molecules and SMIP molecules. In particular, a PIMS molecule was expressed at 5-6 μg/ml, a scorpion was expressed at 1-2 μg/ml and a SMIP molecule was expressed at 10 μg/ml.

### EXAMPLE 4

### ELISA Binding Assay

Comparative ELISA binding assays were performed on a PIMS molecule, a SMIP molecule and a Scorpion molecule. Two capture antibodies were used, i.e., high- and low-affinity anti-CD16 antibodies. To perform the assays, MaxiSorb plates (Costar MaxiSorb black plastic 96-well plates) were each initially coated with 100 μl of 2 μg/ml anti-CD16 low or high affinity antibody (CD16 mIgG high affinity (870 μg/ml): 7.8 μl/3.4 ml PBS; CD16 mIgG low affinity (560 μg/ml): 23.6 μl/6.6 ml PBS). Plates were covered and incubated at 4° C. overnight. The next morning, each plate was washed twice with 200 μl NFDM (PBS/3% non-fat dry milk prepared one day in advance, 3 g/100 ml). Plates were then blocked by adding 200 μl NFDM to each well and incubated for one hour at room temperature. Dilution plates (96-well plastic plates) were made by adding 120 μl NFDM to wells below the highest concentration (C-H for columns 1-7). Subsequently, 120 μl of 8 μg/ml of the protein of interest were added. Proteins of interest (POIs) for CD16 high and low assays: SO129 (an anti-CD20× anti-CD20 multispecific binding protein or scorpion; 1.2 mg/ml) 3.32 μl to 500 μl PBS; 2Lm20-4 (an anti-CD20 SMIP; 1.0 mg/ml—diluted from 54 mg/ml stock), 4 μl to 500 μl PBS; W0001 (an anti-CD28 PIMS; 348 μg/ml) 11.5 μl PBS was then added to a final volume of 500 μl.

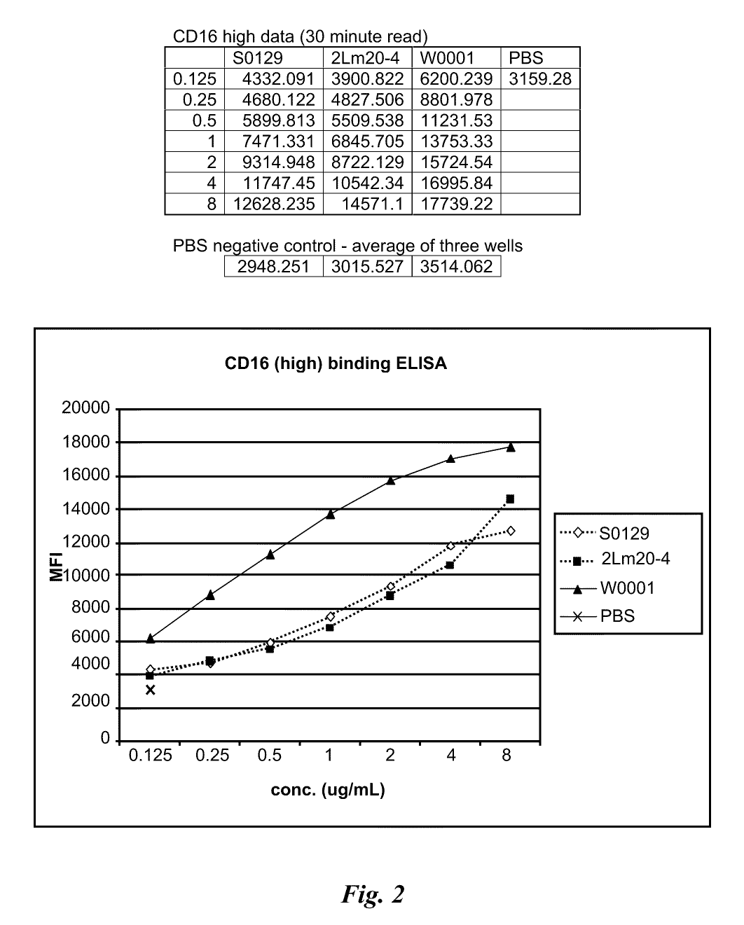

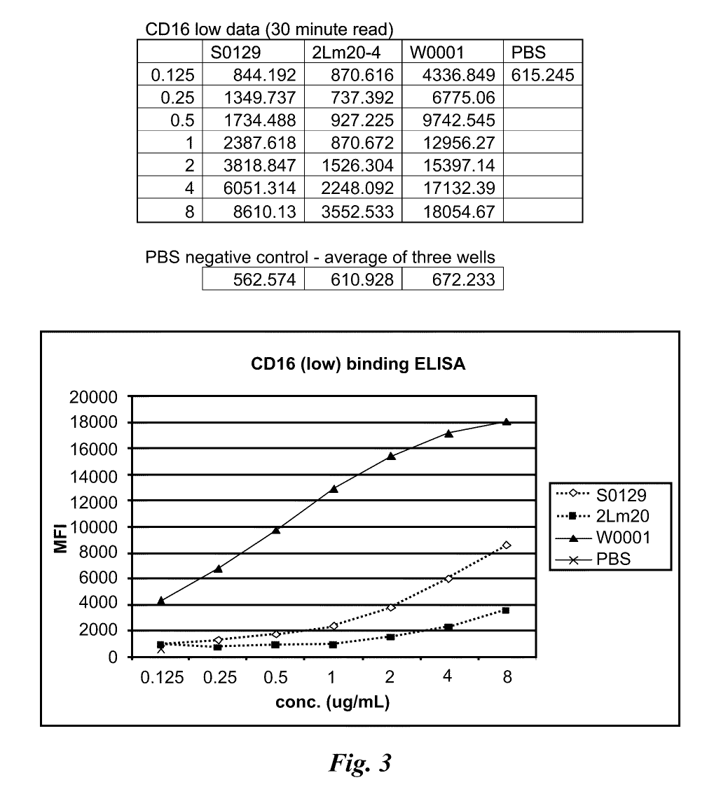

From the mixture in one well, 120 μl was transferred to the next well, and the pattern was continued well-by-well making two-fold serial dilutions. NFDM was then mechanically removed from the MaxiSorb plates (i.e., by flicking). From each well of the dilution plate, 100 μl was transferred to corresponding wells in an ELISA plate. Following transfer, ELISA plates were incubated for one hour at room temperature. During this incubation period, goat anti-human IgG (fcSp) and (H+L)-HRPO conjugates (Caltech code no. H10307, lot no. 14010107, expiration date of January, 2007) were diluted 1:1000 in NFDM (i.e., 10 μl into a final volume of 10 ml NFDM). ELISA plates were then washed three times with PBST (PBS+0.2% Tween20=400 μl Tween 20 to 200 ml PBS) and 100 μl of horseradish peroxidase (HRP) reagent were added to appropriate wells. ELISA plates were then incubated for one hour at room temperature. During this incubation, Pierce Quanta Blue reagent (Pierce Chemicals catalog no. 15169) was prepared by adding 1.1 ml of peroxidase to 8.9 ml of substrate (1:9). ELISA plates were washed three times with PBST and 100 μl of Pierce QuantaBlue mix was added to each well, avoiding bubbles. The wells were then incubated in the dark for 30 minutes at room temperature. A SpectraMAX GeminiXS was then used to measure colorimetric reaction products in plate wells and counts were graphed as mean fluorescence intensity (MFI) as a function of protein concentration. The results are shown in FIG. 2 for high affinity binding and in FIG. 3 for low affinity binding. Both the differences in MFI at the highest protein concentration of a sample source, as well as the differences in how rapidly the signal dropped off with further dilution of the protein sample reflected differences in CD16 binding. The phosphate-buffered saline (PBS) negative control was also plotted to provide the level of background signal in the assay.

### EXAMPLE 5

### Jurkat Cell Binding Assay

Binding studies were performed to assess the specific binding properties of PIMS molecules, such as the W0001 PIMS. Initially, Jurkat cells were plated using conventional techniques. To the seeded multi-well plates, CD28 purified protein was added, using two-fold titrations across the plate from 20 μg/ml down to 0.16 μg/ml. One well containing no protein served as a background control.

Seeded plates containing the proteins were incubated on ice for one hour. Subsequently, the wells were washed once with 200 μl 1% FBS in PBS. Goat anti-human antibody (Fc Sp) labeled with FITC at 1:100 was then added to each well, and the plates were again incubated on ice for one hour. The plates were then washed once with 200 μl 1% FBS in PBS and the cells were re-suspended in 200 μl 1% FBS and analyzed by FACS.

To assess the binding properties of the W0001 anti-CD28 peptide, CD28-expressing Jurkat cells were plated by seeding in individual wells of a culture plate. The CD28 purified protein was then added to individual wells using a two-fold dilution scheme, extending from 20 μg/ml down to 0.16 μg/ml. The W0001 PIMS purified protein was added to individual seeded wells, again using a two-fold dilution scheme, i.e., from 20 μg/ml down to 0.16 μg/ml. One well received no protein to provide a background control. The plates were then incubated on ice for one hour, washed once with 200 μl 1% FBS in PBS, and goat anti-human antibody labeled with FITC (Fc Sp) at 1:100 was added to each well. The plates were again incubated on ice for one hour and subsequently washed once with 200 μl 1% FBS in PBS. Following re-suspension of the cells in 200 μl 1% FBS, FACS analysis was performed. The expressed proteins were shown to bind to CD28 presented on Jurkat cells by flow cytometry (FACS), thereby demonstrating that the W0001 peptide could function to bind the specific target antigen. In addition, the linker used (H1-H6) was not found to significantly affect binding avidity to target antigen.

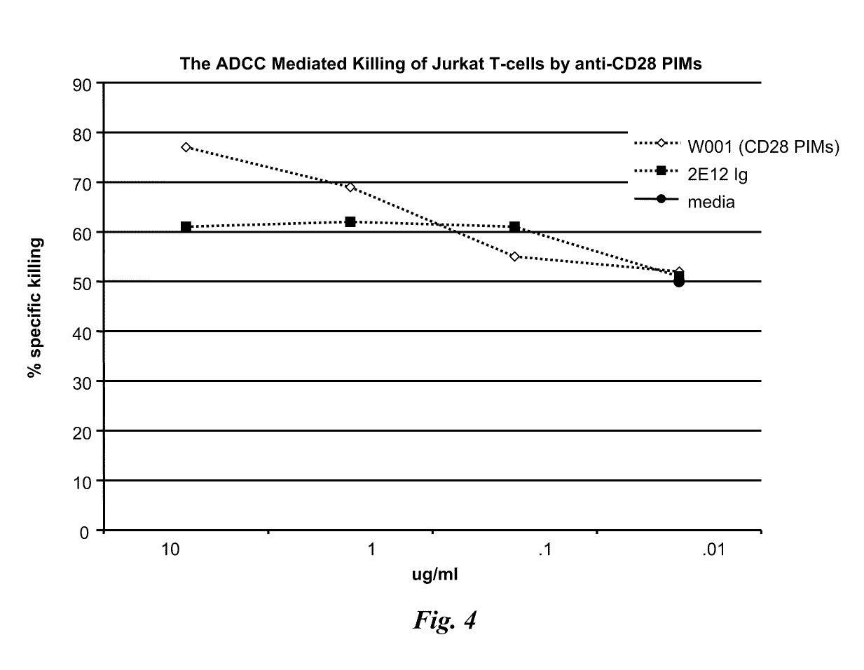

Additionally, the ability of anti-CD28 PIMS and SMIP molecules to mediate ADCC-induced cell death of Jurkat cells using peripheral blood mononuclear cells (PBMCs. Briefly, 1×10⁷/ml Jurkat T-cells were labeled with 500 μCi/ml [⁵¹Cr] sodium chromate (#CJS1, Amersham Biosciences, Piscataway, N.J.) for 90 minutes at 37° C. in Iscoves media (#12440-053, Gibco/Invitrogen, Grand Island, N.Y.) with 10% FBS (#16140-071, Gibco/Invitrogen, Grand Island, N.Y.). The ⁵¹Cr-loaded Jurkat cells were then washed 3 times in RPMI (#11875-093, Gibco/Invitrogen, Grand Island, N.Y.) media with 10% FBS and resuspended at 4×10⁵/ml in RPMI. PBMCs from in-house donors were isolated from heparinized whole blood via centrifugation over Lymphocyte Separation Medium (#50494, MP Biomedicals, Aurora, Ohio), washed 2 times with RPMI media and resuspended at 5×10⁶/ml in RPMI with 10% FBS. Reagent samples were added to RPMI media with 10% FBS at 4 times the final concentration and three 10-fold serial dilutions for each reagent were prepared. These reagents were then added to 96-well U-bottom plates at 501/well for the indicated final concentrations. The ⁵¹Cr labeled Jurkat cells were then added to the plates at 50 μl/well (2×10⁴/well). The PBMC were then added to the plates at 1001/well (5×10⁵/well) for a final ratio of 25:1 effectors (PBMC):target (Jurkat cells). Effectors and targets were added to media alone to measure background killing. The ⁵¹Cr labeled Jurkat cells were added to media alone to measure spontaneous release of ⁵¹Cr and to media with 5% NP40 (#28324, Pierce, Rockford, Ill.) to measure maximal release of ⁵¹Cr. The plates were incubated for 5 hours at 37° C. in 5% CO₂. Fifty μl of the supernatant from each well were then transferred to a LumaPlate-96 (#6006633, Perkin Elmer, Boston, Mass) and dried overnight at room temperature. In the morning, radioactive emissions were measured (cpm) using a Packard TopCount-NXT. Percent specific killing was calculated as follows: ((sample−cpm spontaneous release)/(cpm maximal release-cpm spontaneous release))×100. All units were cpm; samples were the mean of quadruplicate samples. Results presented in FIG. 4 show that the PIMS molecule (W0001) induced or mediated Jurkat cell death.

### EXAMPLE 6

### CD3+ Lymphocyte Binding Assay

A binding study was also performed to assess the specific binding properties of PIMS molecules, such as the W0001 PIMS, to CD3+ lymphocytes. The design of the study involved labeling the CD3+ fraction of a lymphocyte preparation with a phycoerythrin-conjugated murine anti-CD3+ antibody and detecting PIMS, SMIP or background binding to these cells by using a FITC-labeled goat anti-human secondary antibody capable of binding to the constant sub-regions of PIMS and SMIPs.

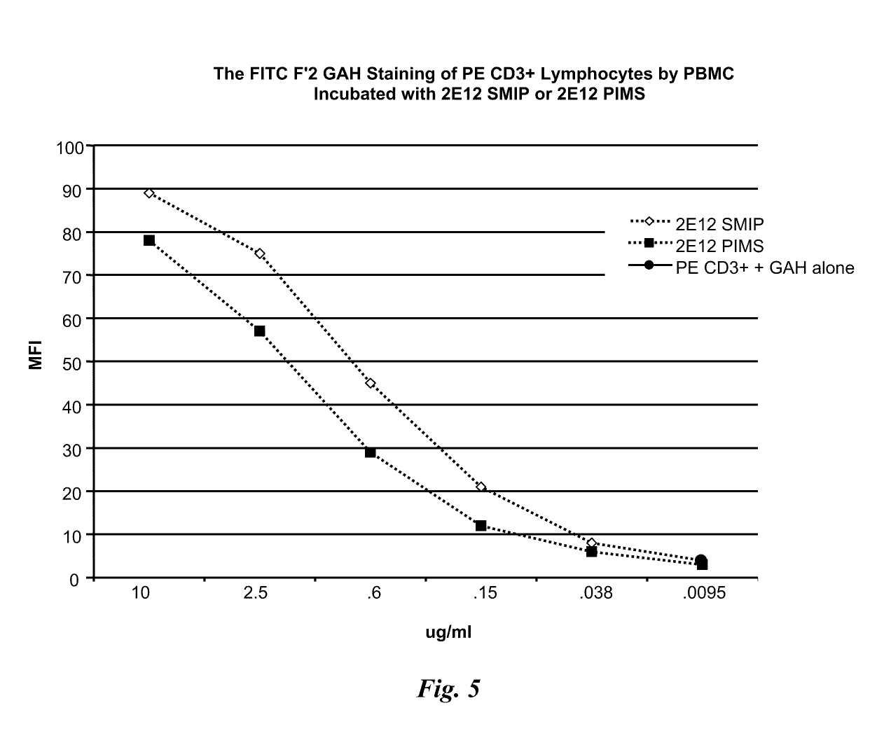

In conducting the experiment, peripheral blood mononuclear cells (PBMCs) were obtained from human donors. The PBMCs were isolated from heparinized whole blood via centrifugation over Lymphocyte Separation Media (MP Biomedicals), washed two times with RPMI media (Gibco) and resuspended at 8×10⁶ cells/ml in staining media (PBS w/2.5% mouse sera/2.5% goat sera). Reagent samples (2E12 SMIP, W0001 (a 2E12 PIMS)) were added to staining media at a concentration of two times the final concentration in the assay and a four-fold dilution series was performed. Sixty microliters per well of reagent samples so treated were plated in a 96-well V-bottom plate (Falcon) and media alone was added to the control well. An appropriate volume of the PBMCs was set aside and PE-conjugated anti-CD3 (BD Pharmingen) was added to these cells to equal 10 μl/well of this reagent. The cells stained with PE (phycoerythrin) anti-CD3 antibody were then added, at 60 μl/well, to the wells containing reagent samples (SMIP, PIMS) or media. The cells were incubated for 45 minutes on ice in the dark. The plates were then washed by centrifugation 2.5 times with cold PBS. (The reference to 2.5 washes actually involves 3 washes, i.e., one wash involving the addition of half of the full volume of PBS to the samples before the first centrifugation, followed by two washes each in a full volume of PBS, as would be understood in the art.) A 1:100 dilution of FITC (fluorescein isothiocyanate)-F′2 Goat anti-Human IgG (Caltag) was then added to the wells in 50 μl of staining media. The cells were incubated for 45 minutes on ice in the dark. The cells were then washed 2.5 times in cold PBS, fixed with 1% paraformaldehyde (USB Corp), stored overnight at 4° C., and read the next day on a FACsCalibur Flow Cytometer and analyzed with Cell Quest software (Becton Dickinson). The results provided in FIG. 5 establish that the mean fluorescence intensity associated with CD3+ lymphocytes increases with increasing concentration of either W0001 (a 2E12 PIMS) or 2E12 SMIP. Thus, the fluorescence intensity is not an artifactual reading and reflects binding of the 2E12 PIMS to CD3+ lymphocytes, further confirming the functional utility of the PIMS protein structure.

### EXAMPLE 7

### Binding Competition

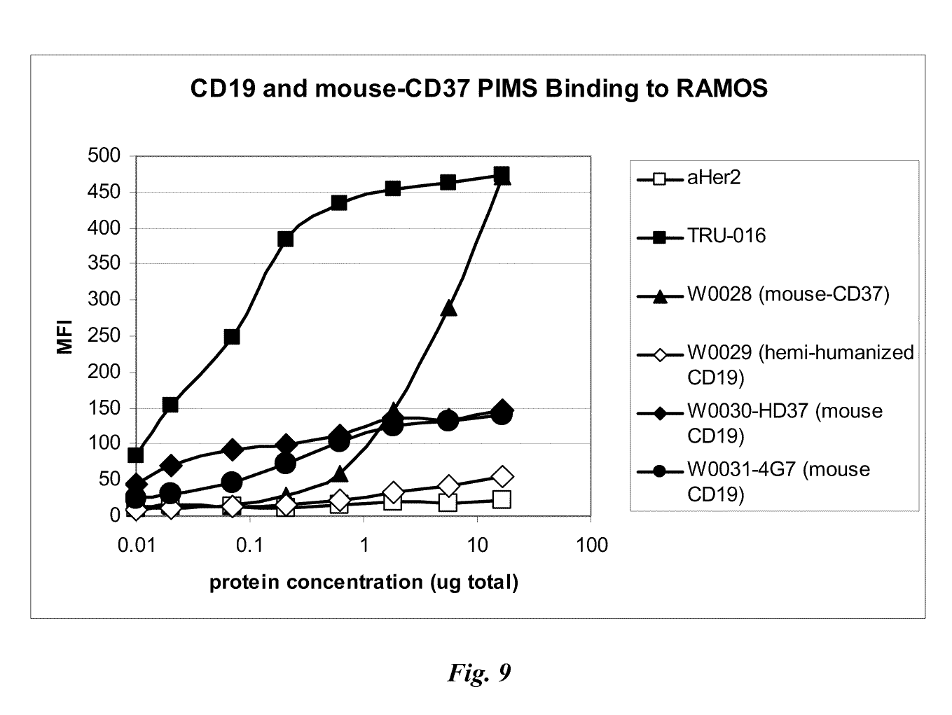

A binding study was conducted to measure the capacity of anti-CD37 PIMS to compete with TRU-016, an anti-CD37 SMIP, for binding to B-cells. Ten mL of RAMOS cells were resuspended in TSA/FBS (1×TSA-50 mM Tris HCl pH 7.8, 0.9% NaCl with 0.5% FBS) to a concentration of 2×10⁶ cells/mL. 100 μl of this cell suspension was added into individual wells of a 96-well, U-bottom plate resulting in 200,000 cells/well. The plate was centrifuged to pellet the cells and the TSA/FBS was removed. Dilutions of competitor proteins were performed beforehand in a dilution plate. The starting concentration of competitor proteins was 1.0 μM and the proteins were serially diluted three-fold. 100 ul of the diluted competitor proteins were added to the wells of the U-bottom plate. 100 uL of 12 nM TRU-016-Eu (Europium-labeled TRU-016) was added to the 100 uL of competitor proteins and cells into each well giving a final concentration of 6 nM TRU-016-Eu in each well. Proteins and cells were incubated at 4° C. for 30 minutes. The treated cells were washed three times with 200 μL TSA/FBS. Cells were resuspended in 200 μL Enhancement solution, transferred to a yellow 96-well plate, shaken for 5 minutes, and then read on an EnVision™ plate reader (PerkinElmer, Waltham, Mass.). The results are shown in FIG. 9. The results showed a displacement of TRU-016-Eu as the concentration of any of the various PIMS molecules increased, regardless of PIMS linker length (10- to 25-amino-acid lengths were examined) and regardless of the type of PIMS linker (H7 or H65). The data establish that a variety of PIMS linkers are functional, as evidenced by the variety of PIMS binding to CD37, with PIMS containing PIMS linkers based on the H65 structure binding better than PIMS containing PIMS linkers based on the H7 linker derived from an immunoglobulin hinge region.

### EXAMPLE 8

### Additional Binding Studies of PIMS

### A. Binding of Anti-CD20 PIMS to Wil2-S Cells

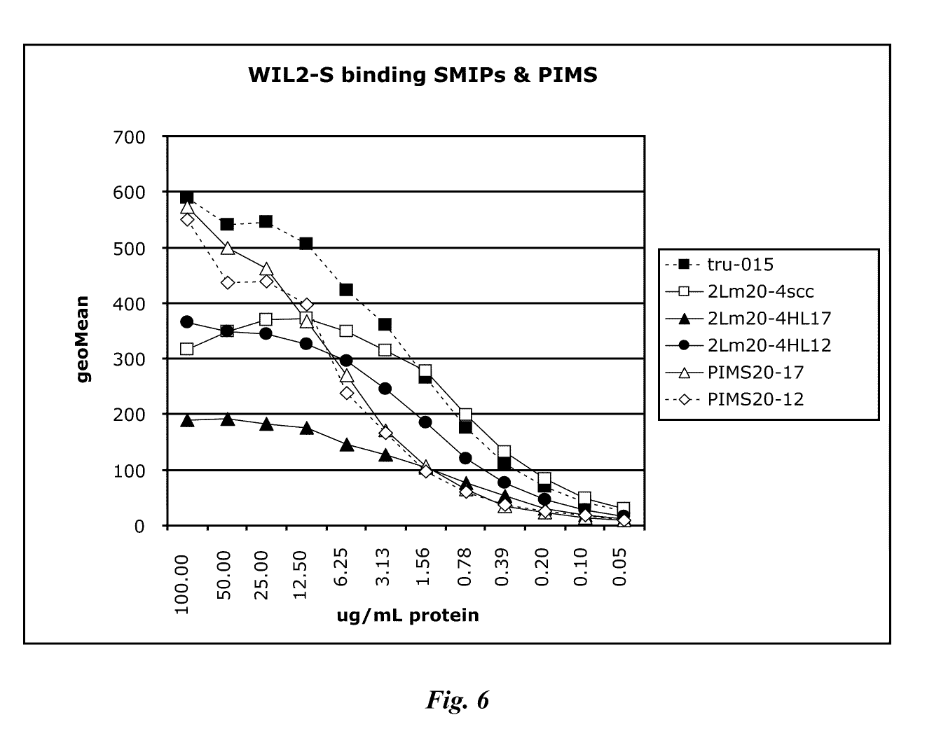

For the Wil2-S B-cell binding study, 100 ug/ml of PIMS was used. In brief, 5×10⁵ Wil2-S B-cells per well were incubated on ice with each of the molecules (e.g., PIMS or SMIP), as indicated in FIG. 6, in FACS buffer (1×PBS, 1% Fetal Bovine Serum, 0.02% sodium azide). Binding was detected using a goat anti-human IgG (gamma specific) conjugated to phycoerythrin (PE) (Jackson Immunoresearch # 109-116-098) at 1:100 dilution in FACS buffer. Results were analyzed by one-color flow cytometry on a FACsCalibur using CellQuest software. In addition to assessing the binding of anti-CD20 PIMS to WIL2-S cells, the binding of anti-CD20 SMIPs to Wil2-S cells expressing CD20 on their surface was also assessed. As noted above, detection of binding was achieved with a fluorescent-labeled secondary antibody that recognizes the Fc portion of SMIPs or PIMS molecules, and results in a fluorescent signal (represented as geoMean), as shown in FIG. 6. In that Figure, 2Lm20-4-scc is an LH SMIP also known as DNE076, 2Lm20-4HL17 is an HL SMIP with a 17-amino-acid gly4ser linker and is also known as DNE079, 2Lm20-4HL12 is an HL SMIP with a 12-amino-acid gly4ser linker and is also known as DNE078, PIMS20-17 is also known as WO009, and PIMS20-12 is also known as W0008.

The results shown in FIG. 6 reveal that higher concentrations of protein exhibited increased signal, representing greater binding that can reach saturation binding (plateau of signal). Both the level of signal achieved and the slope of the graph of signal as a function of protein concentration are indications of binding strength and affinity. These data show that, at lower concentrations, the CD20-directed PIMS bind less well compared to most of the CD20-directed SMIPs; however, at higher concentrations, the signal for PIMS exceeds that from SMIPs. Saturation may be about 2-fold higher for the PIMS molecules compared to the SMIPs with the same CD20 binding domain (2Lm20-4) and configuration (VHVL). In addition, while the gly4ser linker length (12 or 17) affected binding of the HL SMIPs (black circle/triangle), both linker versions of PIMS showed similar binding patterns (open triangle/diamond).

### B. Binding of Anti-DR PIMS to Wil2-S Cells

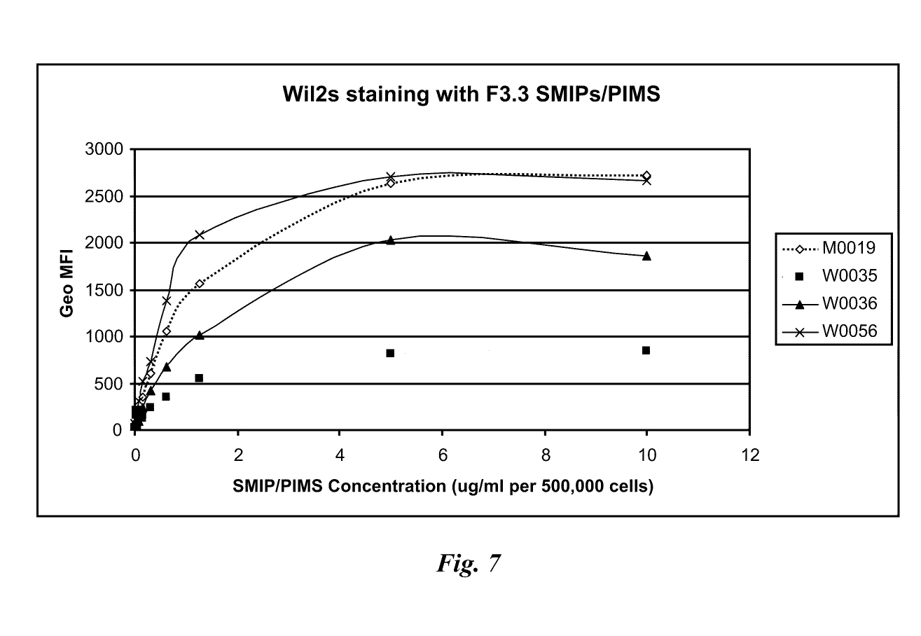

A binding study was also conducted to assess the capacity of anti-DR PIMS molecules to bind to Wil2-S B-cells. The binding assay described above was used with appropriate substitutions of proteins of interest. In brief, 500,000 Wil2-S cells were placed in each well of a multi-well plate and were incubated on ice with one of the PIMS or SMIPs under investigation in FACS buffer (1×PBS, 1% FBS, 0.02% sodium azide). Detection of phycoerythrin was achieved following exposure of cells to 1:100 dilution of PE-conjugated goat anti-human IgG (gamma specific) secondary antibody (Jackson Immunoresearch #109-116-098) in FACS buffer. The results, presented in FIG. 7, show that the binding of anti-DR PIMS molecules is strongly dependent on the PIMS linkers. W0035, the anti-DR PIMS with the H7 linker, has the lowest binding activity. W0036, another anti-DR PIMS with the H62 linker, binds better than W0035. W0056, containing the H65 linker, exhibited the best binding activity, comparable to the parental anti-DR SMIP (M0019).

### C. Binding of Anti-CD37 PIMS and Anti-CD19 PIMS to Ramos Cells

Another binding study assessed the capacities of mouse anti-CD37 PIMS and anti-CD19 PIMS to bind to Ramos B-cells. The assay was performed as generally described above and in Example 5, and the results are presented in FIG. 9. The proteins under investigation in this experiment were aHer2 (anti-Her2). See Example 12 for disclosure relating to Her2), TRU-016 (an anti-CD37 SMIP), W0028 (a mouse anti-CD37 PIMS), WO029 (a hemi-humanized anti-CD19 SMIP), W0030 (a mouse anti-CD19 PIMS, and W0031 (a different mouse anti-CD19 PIMS). For each protein, 3:1 serial dilutions were prepared spanning the range of 16.7 ug to 0.01 ug. The results in FIG. 9 show tha the TRU-016 SMIP and W0028 bind in significantly greater quantity than the other PIMS or aHer2 as the concentration of the protein increases from 0.01 ug/ml, but FIG. 9 also establishes that the various PIMS proteins being tested bound to their targets.

### D. Binding of Anti-CD28 PIMS to Jurkat T-Cells

The binding of anti-CD28 PIMS to Jurkat T-cells was also investigated. A variety of anti-CD28 PIMS proteins were analyzed, i.e., W0001 (an anti-CD28 PIMS with the H7 PIMS linker), W0050 (an anti-CD28 PIMS with the H9 PIMS linker), W0051 (an anti-CD28 PIMS with the H47 PIMS linker), W0052 (an anti-CD28 PIMS with the H56 PIMS linker), W0053 (an anti-CD28 PIMS with the H62 PIMS linker), WO083 (an anti-CD28 PIMS with the H65 PIMS linker), and an anti-CD28 SMIP. To conduct this binding study, for each of the above-mentioned proteins, 50 ul of protein solution at varying concentrations from 10 ug/ml to 5 ng/ml were individually added to wells of a V-shaped 96-well plate. Next, 2.5×10⁵ Jurkat cells in 50 ul were added to each well. Samples were then incubated on ice for 30 minutes, washed 2 times with 1% BSA in PBS and a 1:200 dilution of anti-human IgG-PE in 1% BSA in PBS was added. The plate was incubated on ice for an additional 30 minutes and washed once with 1% BSA in PBS. Cells were resuspended in 2% formaldehyde in PBS. The mean fluorescence intensity of binding in each well was measured using a Facscan.

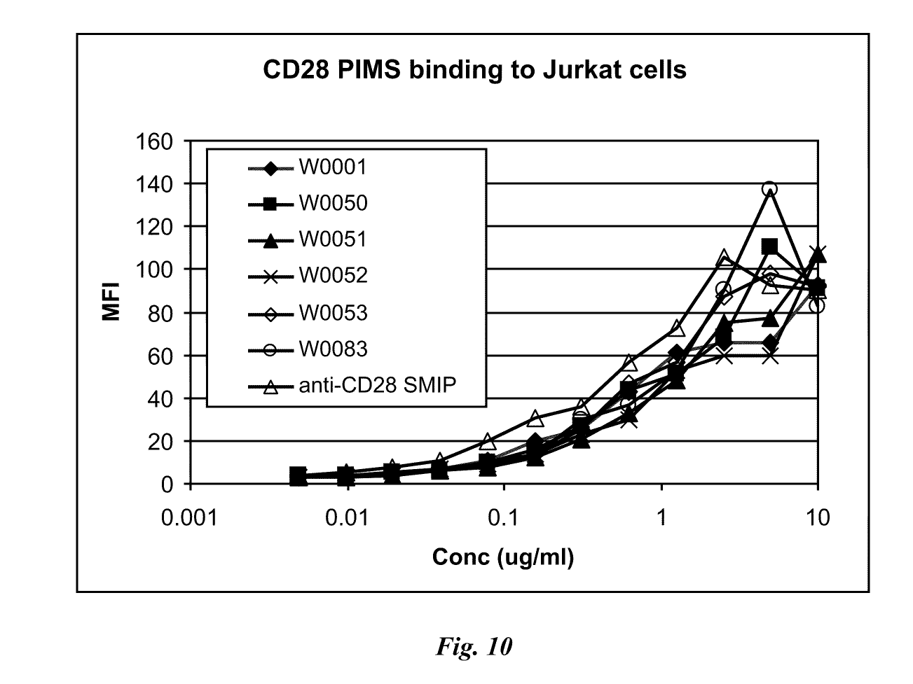

The results of the binding study involving anti-CD28 PIMS and Jurkat T-cells are shown in FIG. 10. All of the anti-CD28 PIMS, regardless of the particular PIMS linker, exhibited comparable binding activity. PIMS linkers did not appeared to influence anti-CD28 PIMS binding to Jurkat cells. This is unlike the case of anti-DR PIMS binding, where binding is strongly influenced by the type of scorpion linker being used.

### E. Binding of PIMS Constant Sub-Regions to CD16

A binding study of the constant sub-region of PIMS molecules was also conducted using CD16, identified as F_{C} receptors F_{C}γRIIIa and F_{C}γRIIIb. CD16 binds to the F_{C} region of IgG antibodies. To assess the binding properties of PIMS to CD16, a low-affinity CD16 was employed. To conduct the assay, Ramos cells were added to cell culture wells at 350,000 cells/well. Solutions of the proteins of interest, including TRU-016 (an anti-CD37 SMIP) and anti-CD37 PIMS molecules were added at concentrations ranging from 0.011 ug/well to 1.2 ug/well; CD16 was added to 1 ug/well. Reaction mixtures were then washed 2.5× with 200 ul FACS buffer (1×PBS, 1% FBS, 0.02% sodium azide). Goat anti-mouse conjugated to phycoerythrin (PE) at 1:100 dilution (Jackson Immunoresearch # 115-116-071) was then added and the mixture was incubated on ice for 45 minutes. Subsequently, the reaction mixtures were washed 1.5× with FACS buffer and subjected to analysis. The CD16lo (low affinity CD16) binding data is presented in Table 9.

TABLE 9
Geo
MFI

| Sample | | | | | | | | | |
|---|---|---|---|---|---|---|---|---|---|
| conc (ug/ml) | TRU016 | W0012 | W0023 | W0024 | W0025 | W0094 | W0095 | W0096 | W0097 |
| 24 | 157.72 | 119.99 | 75.33 | 145.82 | 124.11 | 77.19 | 85.82 | 65.36 | 64.89 |
| 8 | 227.94 | 69.76 | 41.97 | 102.88 | 81.76 | 105.02 | 89.6 | 99.77 | 96.44 |
| 2.67 | 228.76 | 30.74 | 17.71 | 44.38 | 32.29 | 98.89 | 59.99 | 93.57 | 97.8 |
| 0.89 | 214.19 | 11.73 | 6.67 | 16.38 | 10.27 | 52.42 | 24.32 | 43.65 | 54.55 |
| 0.3 | 143.31 | 4.25 | 2.86 | 5.17 | 3 | 16.69 | 5.1 | 11.53 | 20.39 |
| 0.1 | 57.46 | 2.25 | 1.7 | 2.03 | 1.61 | 3.88 | 1.64 | 2.71 | 6.12 |
| 0.033 | 21.75 | 1.59 | 1.45 | 1.49 | 1.31 | 1.57 | 1.36 | 1.46 | 2.02 |
| 0.011 | 7.41 | 1.34 | 1.38 | 1.33 | 1.42 | 1.28 | 1.19 | 1.25 | 1.61 |

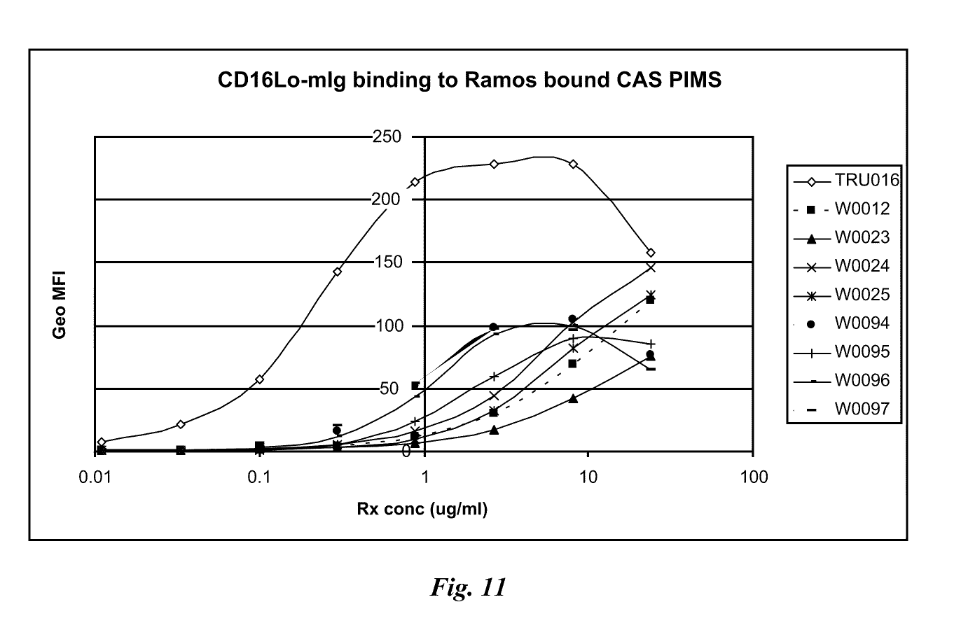

A graphic illustration of the results of the binding study involving CD16lo binding to anti-CD37 PIMS molecules and controls is presented in FIG. 11.

All of the anti-CD37 PIMS subjected to analysis, which included PIMS with a wide variety of PIMS linkers, showed lower CD16 binding compared to TRU-016 (anti-CD37 SMIP). These findings are consistent with the ADCC assay results described in Example 9 below.

### EXAMPLE 9

### ADCC Activity of PIMS

To assess the antibody-dependent cellular cytotoxicity (ADCC) inducible by, or mediated by, PIMS, ADCC assays were conducted. Briefly, 1×10⁷ cells/ml BJAB B-cells were labeled with 500 uCi/ml ⁵¹Cr sodium chromate (#CJS1, Amersham Biosciences, Piscataway, N.J.) for 2 hours at 37° C. in Iscoves media (#12440-053, Gibco/Invitrogen, Grand Island, N.Y.) with 10% FBS (#16140-071, Gibco/Invitrogen, Grand Island, N.Y.). The ⁵¹Cr-loaded BJAB B-cells were then washed 3 times in RPMI (#11875-093, Gibco/Invitrogen, Grand Island, N.Y.) media with 10% FBS and resuspended at 4×10⁵ cells/ml in RPMI. Peripheral blood mononuclear cells (PBMC) from in-house donors were isolated from heparinized whole blood via centrifugation over Lymphocyte Separation Medium (#50494, MP Biomedicals, Aurora, Ohio), washed 2 times with RPMI media and resuspended at 5×10⁶ cells/ml in RPMI with 10% FBS. Reagent samples were added to RPMI media with 10% FBS at 4 times the final concentration and three 10-fold serial dilutions for each reagent were prepared. These reagents were then added to 96-well U-bottom plates at 50 ul/well to achieve the indicated final concentrations. The ⁵¹Cr-labeled BJAB cells were then added to the plates at 50 ul/well (2×10⁴ cells/well).

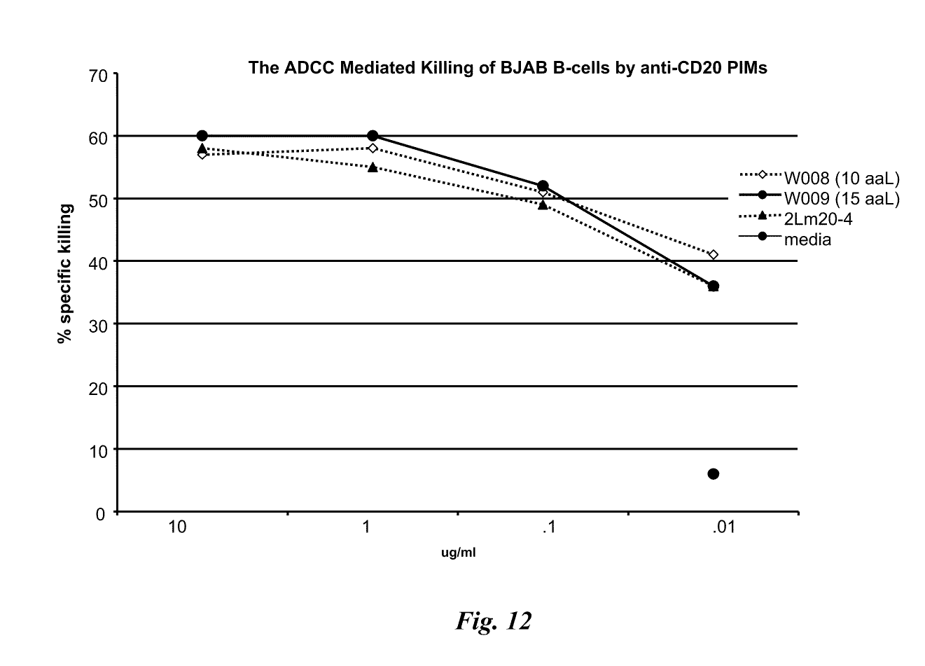

The PBMC were then added to the plates at 100 ul/well (5×10⁵ cells/well) for a final ratio of 25:1 effectors (PBMC):target (BJAB). Effectors and targets were added to media alone to measure background killing. The ⁵¹Cr-labeled BJAB B-cells were added to media alone to measure spontaneous release of ⁵¹Cr and to media with 5% NP40 (#28324, Pierce, Rockford, Ill.) to measure maximal release of ⁵¹Cr. The plates were incubated for 6 hours at 37° C. in 5% CO₂. Fifty ul of the supernatant from each well were then transferred to a LumaPlate-96 (#6006633, Perkin Elmer, Boston, Mass) and dried overnight at room temperature. In the morning, cpm were read on a Packard TopCount-NXT. Percent specific killing was calculated according to the following equation: ((cpm of sample (mean of quadruplicate set of samples)−cpm spontaneous release)/(cpm maximal release-cpm spontaneous release))×100. Results are shown in FIG. 12, which reveals a general increase from about 40% to about 58% in the percentage of specifically killed BJAB B-cells as protein concentration is increased from 0.01 ug/ml to 10 ug/ml for each of the tested proteins, i.e., 2Lm20-4 (a humanized anti-CD20 SMIP), W0008 (an anti-CD20 PIMS with a 10-amino-acid PIMS linker), and W0009 (an anti-CD20 PIMS with a 15-amino-acid PIMS linker). As expected, the media control showed little cell killing.

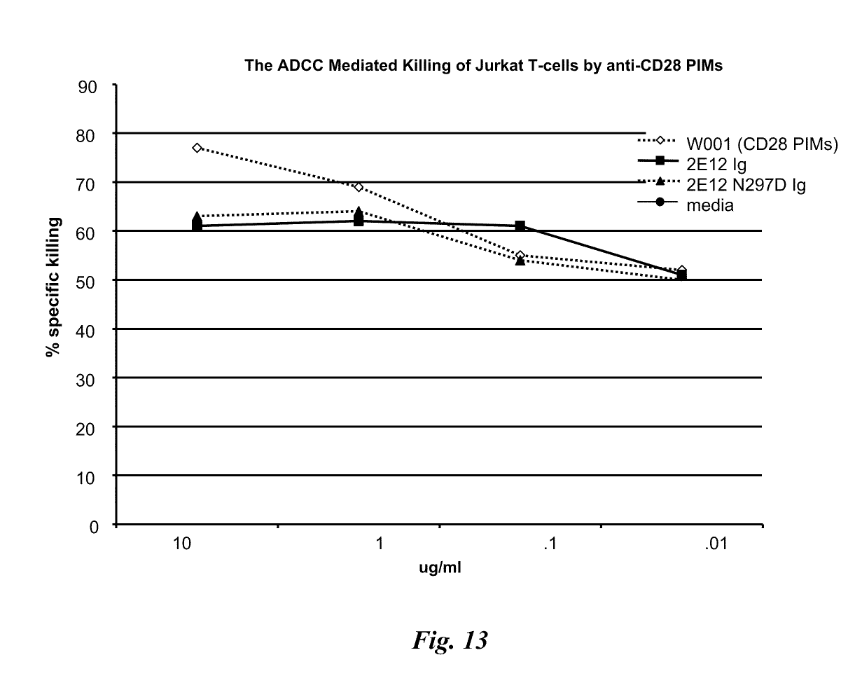

The capacity of anti-CD28 PIMS molecules to induce ADCC-mediated killing of Jurkat T-cells was assessed in an ADCC assay as described above. The results are shown in FIG. 13, which revealed that W001, an anti-CD28 PIMS, induced a greater percentage of specific killing of Jurkat T-cells as the concentration of that protein increased than was found with either of two anti-CD28 SMIP molecules (2E121g or a variant thereof in the form of 2E12 N297D Ig) or with media alone.

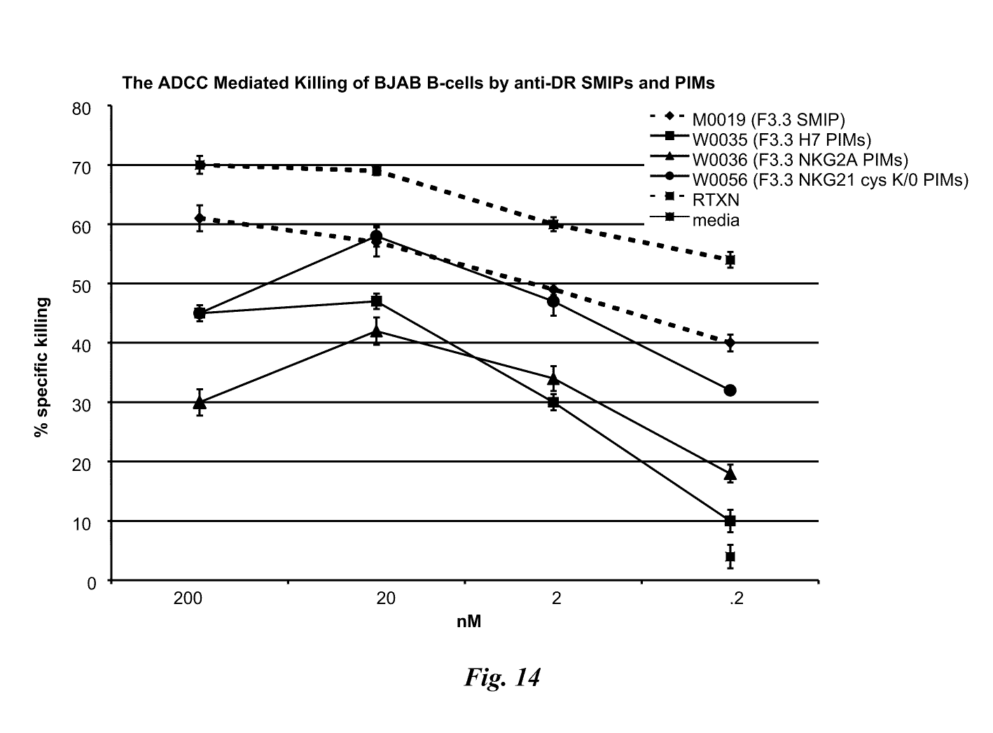

An analogous ADCC assay was conducted to determine the capacity of anti-DR PIMS to induce ADCC of BJAB B-cells. The assay was again conducted as described above, and the results are shown in FIG. 14. Rituximab showed the highest percentage of specific cell killing at all tested concentrations, and the percentage of specifically killed cells increased with increasing concentrations of the protein. Similarly, M0019, an anti-DR SMIP (with an H7 linker region) showed a high level of specific cell killing and the percentage of killed cells increased with each increase in protein concentration. The anti-DR PIMS also showed specific cell killing, with the percentage of killed cells increasing as the percentage of PIMS protein increased from 0.2 nM to 20 nM. W0056, an anti-DR PIMS (containing an H65 PIMS linker) exhibited the highest level of specific cell killing of any anti-DR PIMS at all tested concentrations. W0036 (anti-DR PIMS with an H62 PIMS linker), like W0056, showed a decrease in percentage of specific cell killing as the relevant PIMS concentration was increased from 20 to 200 nM. In contrast, W0035 (anti-DR PIMS with an H7 PIMS linker) exhibited a dramatic increase in percentage of specifically killed cells as the PIMS concentration increased from 0.2 to 20 nM and then the percent of killed cells roughly plateaued as the concentration was increased beyond 20 nM to 200 nM. The results demonstrate that anti-DR PIMS can induce 42-58% ADCC-mediated cell death of BJAB B-cells at a PIMS concentration of 20 nM, with the higher end of the range attributable to PIMS with the H65 PIMS linker and the lower end of the range attributable to PIMS with the H7 PIMS linker.

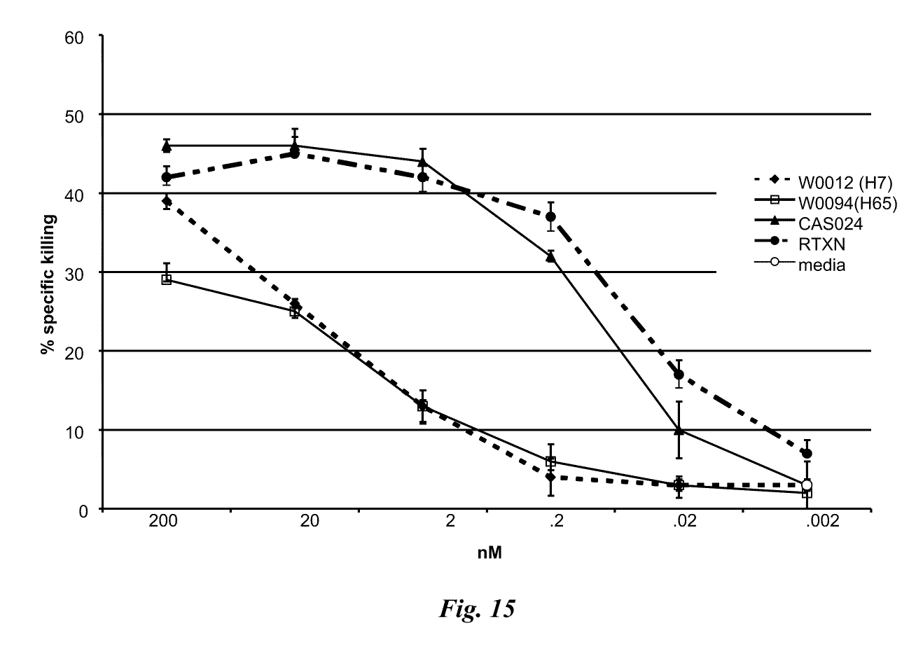

An ADCC assay as described above was also performed to assess the capacity of anti-CD37 PIMS to induce the ADCC-mediated cell death of BJAB B-cells. In this assay, two PIMS (W0012 and W0094) were compared with TRU-016 (an anti-CD37 SMIP). W0012 is an anti-CD37 PIMS with an H7 PIMS linker. W0094 is an anti-CD37 PIMS with an H65 PIMS linker. Also assessed in this assay were rituximab as a positive control and media alone as a negative control. The results showed that both PIMS had lower ADCC activity than the SMIP, as shown in FIG. 15. Although the binding studies disclosed herein showed that W0094 bound better than W0012, W0094 does not exhibit greater ADCC inducing capacity than W0012, unlike the results disclosed above for anti-DR PIMS, where anti-DR PIMS with the H65 PIMS linker showed consistently greater ADCC activity than anti-DR PIMS with the H7 PIMS linker.

### EXAMPLE 10

### CDC Activity of PIMS

Complement-dependent cytotoxicity (CDC) provides another mechanism by which eukaryotic (e.g., mammalian) cells such as B-cells are killed. The CDC activity of PIMS was explored to determine whether these single-chain molecules exhibiting specific target binding could also induce, or mediate, CDC of target cells, such as B-cells expressing a PIMS binding partner on their surface. To assess CDC activity, 5 to 2.5×10⁵ Ramos B-cells were added per well to 96-well V-bottomed plates in 50 ul of Iscoves (#12440-053, Gibco/Invitrogen, Grand Island, N.Y.) media (no FBS). The proteins subjected to the assay were 2Lm20-4 (a humanized anti-CD20 SMIP), TRU-015 (an anti-CD20 SMIP), W0008 (a PIMS having a binding domain in HL orientation with a 10-amino-acid PIMS linker), W0009 (a PIMS having a binding domain in HL orientation with a 15-amino-acid PIMS linker) and, as a negative control, media alone. Separately, each of these proteins in Iscoves, (or Iscoves alone) was added to the wells in 50 ul at 2 times the indicated final concentration. The cells and reagents were incubated for 45 minutes at 37° C. The cells were washed 2½ times in Iscoves media with no FBS and resuspended in Iscoves with human serum (# A113, Quidel, San Diego, Calif.) in the 96-well plate at the indicated concentrations. The cells were then incubated for 90 minutes at 37° C. The cells were washed by centrifugation and resuspended in 125 ul cold PBS. The cells were transferred to FACs cluster tubes (#4410, CoStar, Corning, N.Y.) and 125 ul PBS with propidium iodide (# P-16063, Molecular Probes, Eugene, Oreg.) at 5 ug/ml was added. The cells were incubated with propidium iodide for 15 minutes at room temperature in the dark and then placed on ice and read and analyzed on a FACsCalibur with CellQuest software (Becton Dickinson).

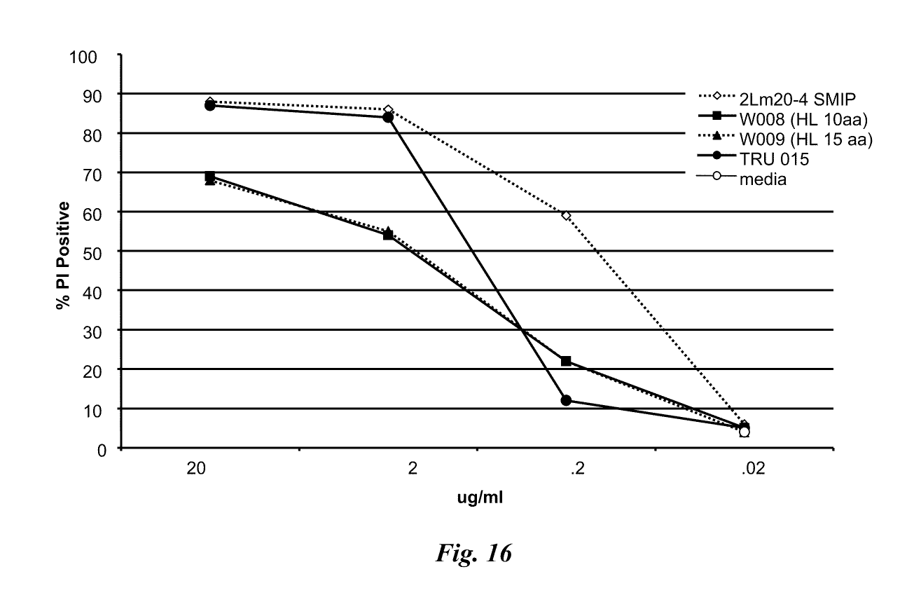

The results are shown in FIG. 16. Apparent from the Figure, as the concentration of protein increased, the CDC activity of each of the two SMIPs increased from about 5% PI-positive cells at 0.2 ug/ml protein to about 88% PI-positive cells at 20 ug/ml. The two PIMS molecules also showed about 5% PI-positive cells at 0.2 ug/ml, but rose to about 69% PI-positive cells at 20 ug/ml. The percentage of PI-positive cells, a measure of the level of CDC activity induced, remained virtually identical for the two PIMS molecules at all tested concentrations, indicating that PIMS linkers of 10-15 amino acids function similarly with respect to CDC induction.

### EXAMPLE 11

### Cell Growth Inhibition by PIMS

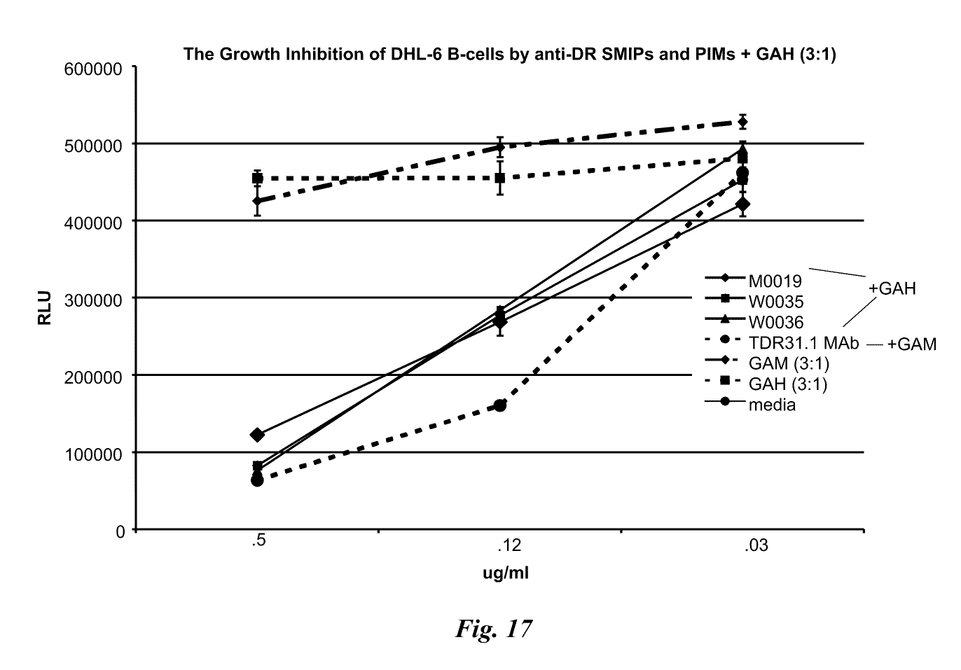

The preceding examples established that PIMS molecules are useful in inducing cell death by ADCC and/or CDC. In addition, PIMS molecules are useful in inhibiting the growth of eukaryotic cells. To establish this property of PIMS molecules, four-fold dilutions of various PIMS proteins, SMIP proteins, and other controls were prepared in RPMI 1640 (Gibco Invitrogen #11875, Grand Island, N.Y.) with 10% FCS (Gibco/Invitrogen #01-40200J, Grand Island, N.Y.) to a concentration of four times the final concentration shown in FIG. 17. SU-DHL-6 B-cells (DMSZ # ACC 572, Braunschweig, Germany) were suspended in the RPMI with 10% FCS to a concentration of 2×10⁵ cells/ml. Black 96-well flat-bottom plates were used for the assay and media was added to wells where necessary to result in a final well volume of 200 microliters. Cells were then added at 50 microliters/well (10⁴ cells/well). The proteins of interest were then separately added to the wells at 50 microliters/well. Cross-linking solutions of Fab′2 Goat anti-Mouse IgG (GAM; Jackson Immunoresearch Labs #115-006-062, West Grove, Pa.) or Fab′2 Goat anti-Human IgG (GAH; Jackson Immunoresearch Labs #109-006-008, West Grove, Pa.) in media were prepared. Four-fold dilutions were prepared to yield a final concentration of cross-linker at three times the concentration of the proteins of interest. These proteins were then separately added to the wells with the goat anti-human secondary antibody added to the wells with PIMs and SMIP and the goat anti-mouse secondary antibody was added to the wells with the monoclonal antibody (see FIG. 17). Plates were incubated for 72 hours at 37° C. in 5% CO₂.

The impact of the various proteins of interest on ATP release was measured by ATPlite (Perkin Elmer # 6016943, Waltham, Mass.). These cytotoxicity studies were performed as recommended by the manufacturer utilizing a substrate solution that emits light in a manner proportional to the ATP present in each sample. Briefly, mammalian cell lysis buffer was added to lyse the cells, followed by addition of the substrate solution. The amount of light produced in each well was measured in a TopCountR Microplate Scintillation and Luminescence Counter (Perkin Elmer, Waltham, Mass.). The results shown in FIG. 17 are represented as the mean and standard deviation of quadruplicate samples. The results demonstrate that, as PIMS (or SMIP) concentration is increased from 0.03 ug/ml to 0.5 ug/ml, growth inhibition of DHL-6 B-cells dramatically increases relative to the more modest growth inhibition seen with the monoclonal antibodies tested at the same protein concentrations. Thus, PIMS exhibit the capacity to inhibit cell growth.

### EXAMPLE 12

### Anti-Her2 PIMS

Her2 (also known as neu, ErbB-2, and ERBB2) is a protein associated with aggressive breast cancers. The protein is a member of the ErbB protein family, or the epidermal growth factor receptor family. It is a cell membrane surface-bound receptor tyrosine kinase that is normally involved in the signal transduction pathways leading to cell growth and differentiation, and it has been identified as a target for anti-cancer treatments, such as treatments for breast cancer, ovarian cancer, stomach cancer, and others. A PIMS molecule specifically recognizing Her2 would be expected to target the ADCC, CDC and growth inhibitory properties of PIMS to cells expressing Her2 at high levels, i.e., to cancer cells.

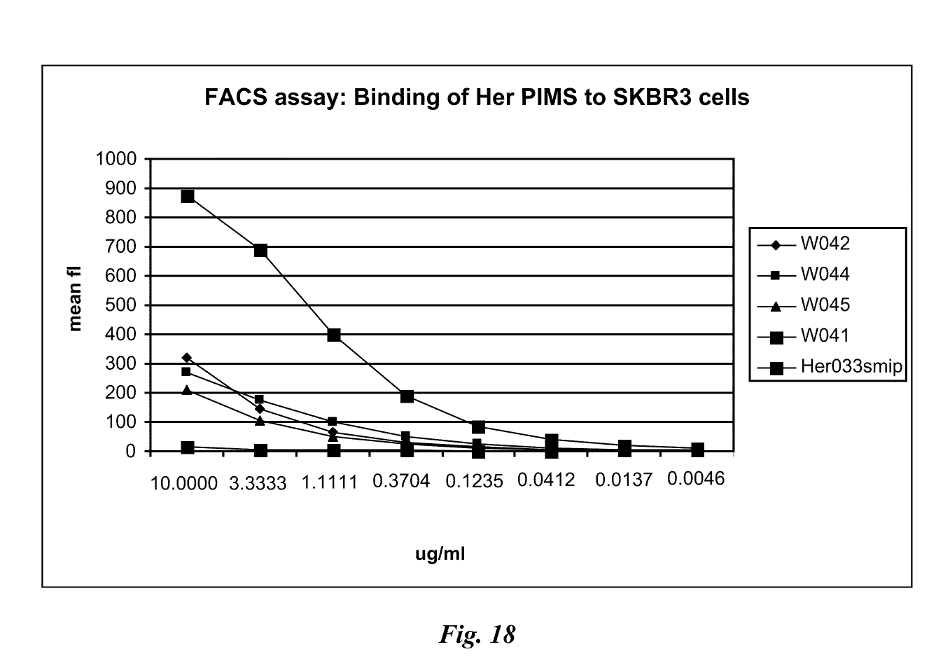

To assess the capacity of PIMS to recognize Her2, a binding assay was performed using the protocol described in Examples 5 and 6, with appropriate substitution of anti-Her2 PIMS and SKBR3 breast cancer cells expressing Her2. The results are shown in FIG. 18, which shows that Her033smip (i.e., an anti-Her2 SMIP) exhibited a dramatic increase in mean fluorescence intensity as the protein concentration was increased from 0.0046 ug/ml to 10.0000 ug/ml. Less dramatic, but still significant, increases in mean fluorescence intensity as protein concentration increased were seen with W042, W044 and W045, three anti-Her2 PIMS molecules. The W041 PIMS did not appear to bind to Her2 on SKBR3 cells. The data establish that anti-Her2 PIMS molecules do bind Her2 on the surface of SKBR3 cells.

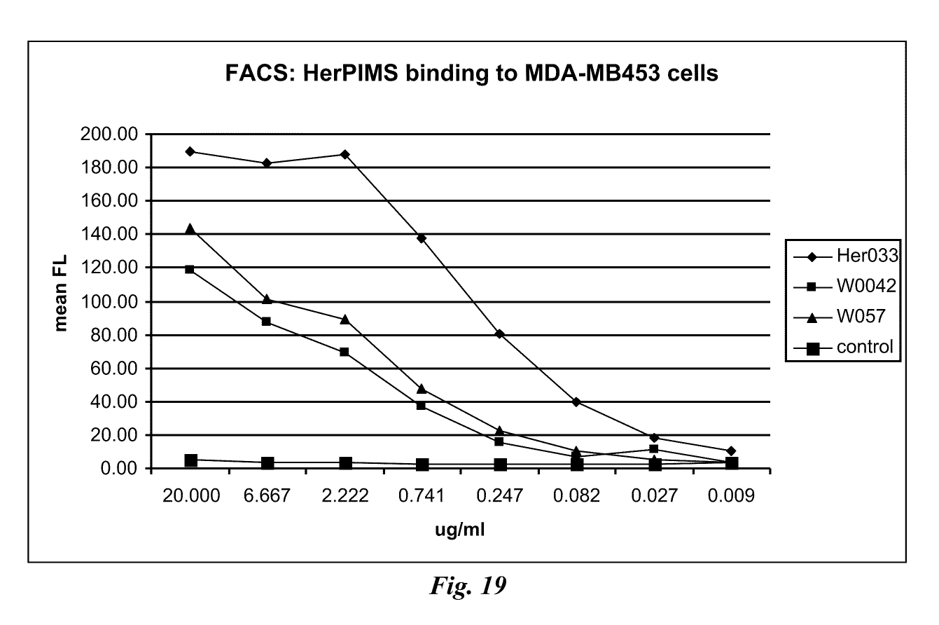

The results obtained with SKBR3 cells were also found when Her2 PIMS were exposed to another breast cancer cell line, the MDA-MB453 cell line. With appropriate substitutions of proteins of interest and cells, the protocol described above and in Examples 5 and 6 was followed. Results are presented in FIG. 19, which shows the mean fluorescent intensity for Her033, the anti-Her2 SMIP, rose rapidly with increasing protein concentration and then plateaued between 2.222 and 20.000 ug/ml. The W0042 and W0057 PIMS molecules also showed significance increase in binding to MDA-MB453 cells, as revealed by the marked increase in mean fluorescent intensity with increasing protein concentration. No plateau was seen for PIMS binding. As expected, the control exhibited negligible binding as shown by the minimal mean fluorescent intensity observed through all tested concentrations.

The binding of Her2 displayed on multiple breast cancer cell lines by PIMS molecules indicates that PIMS will be useful in cancer diagnosis, prognosis and treatment, including but not limited to cancers associated with Her2 expression or over-expression, such as breast, ovarian and stomach cancers. More generally, PIMS that target a cancer marker are expected to be useful diagnostic, prognostic and therapeutic agents.

Variations on the structural themes for specific binding proteins with effector function will be apparent to those of skill in the art upon review of the present disclosure, and such variant structures are within the scope of the invention.

What is claimed is:

1. A specific binding protein comprising:
a constant sub-region derived from an antibody;
a PIMS linker disposed C-terminal to the constant sub-region; and
a specific binding domain comprising at least one of a V_{L} domain and a V_{H} domain, the specific binding domain disposed C-terminal to the PIMS linker, wherein the specific binding protein specifically binds at least one target and exhibits at least one effector function of an antibody molecule.

2. The specific binding protein according to claim 1 wherein the constant sub-region comprises a C_{H2} domain and a C_{H3} domain, wherein at least one of the C_{H2} domain and the C_{H3} domain is a complete antibody domain.

3. The specific binding protein according to claim 2 wherein the antibody domain is selected from the group consisting of IgG, IgE, IgD, IgA and IgM antibody domains.

4. The specific binding protein according to claim 2 wherein at least one of the C_{H2} domain and the C_{H3} domain comprises a sequence selected from the group consisting of SEQ ID NO:377 and SEQ ID NO:379.

5. The specific binding protein according to claim 1 wherein the effector function is antibody-dependent cellular cytotoxicity or complement-mediated cytotoxicity.

6. The specific binding protein according to claim 1 wherein the PIMS linker is derived from a stalk region of a type II C-lectin.

7. The specific binding protein according to claim 1 wherein the PIMS linker is selected from the group consisting of an antibody hinge region, a CD72 stalk region, NKG2a and NKG2a C18S.

8. The specific binding protein according to claim 1 wherein the PIMS linker is an antibody hinge region selected from the group consisting of IgG, IgA, IgD, IgE hinges and variants thereof.

9. The specific binding protein according to claim 8 wherein the hinge is an antibody hinge region selected from the group consisting of human IgG1, human IgG2, human IgG3, human IgG4, and human variants thereof.

10. The specific binding protein according to claim 1 wherein the PIMS linker has a single cysteine residue for formation of an interchain disulfide bond.

11. The specific binding protein according to claim 1 wherein the PIMS linker has two cysteine residues for formation of interchain disulfide bonds.

12. The specific binding protein according to claim 1 wherein the PIMS linker comprises a sequence selected from the group consisting of SEQ ID NOS:61-118.

13. The specific binding protein according to claim 1 wherein the protein specifically binds a target selected from the group consisting of CD3, CD19, CD20, CD28, CD37 and DR.

14. The specific binding protein according to claim 1 wherein the protein is selected from the group consisting of W0001, W0002, W0003, W0004, W0005, W0006, W0007, W0008, W0009, W0011, W0012, W0023, W0024, W0025, W0028, W0029, W0030, W0031, W0035, W0036, W0041, W0042, W0044, W0045, W0050, W0051, W0052, W0053, W0055, W0056, W0057, W0083, W0087, W0094, W0095, W0096 and W0097.

15. The specific binding protein according to claim 1 wherein the V_{L} domain and the V_{H} domain are separated by an interdomain linker.

16. The specific binding protein according to claim 15 wherein the structure of the interdomain linker is (Gly₄Ser)ₙ, where n=1-5.

17. The specific binding protein according to claim 15 wherein the interdomain linker comprises a sequence selected from the group consisting of SEQ ID NO:544, SEQ ID NO:545, SEQ ID NO:184, SEQ ID NO:240, SEQ ID NO:242, SEQ ID NO:243, SEQ ID NO:245, SEQ ID NO:247, SEQ ID NO:248, SEQ ID NO:539 and SEQ ID NO:540.

18. The specific binding protein according to claim 1 wherein at least one of the V_{L} domain and the V_{H} domain comprises a sequence selected from the group consisting of residues 23-128 of SEQ ID NO:2, residues 145-265 of SEQ ID NO:2, residues 520-640 of SEQ ID NO:2, residues 661-772 of SEQ ID NO:2, residues 508-629 of SEQ ID NO:28, residues 647-754 of SEQ ID NO:28, residues 508-629 of SEQ ID NO:30, residues 652-759 of SEQ ID NO:30, residues 21-127 of SEQ ID NO:44, residues 143-264 of SEQ ID NO:44, residues 1-121 of SEQ ID NO:354 and residues 134-239 of SEQ ID NO:354.

19. The specific binding protein according to claim 1 further comprising a hinge disposed N-terminal to the sub-region.

20. The specific binding protein according to claim 19 wherein the hinge comprises the same sequence as the PIMS linker disposed between the constant sub-region and the specific binding domain.

21. The specific binding protein according to claim 1, further comprising at least a second specific binding domain disposed C-terminal to the constant sub-region.

22. The specific binding protein according to claim 21, wherein each of the specific binding domains binds to the same target.

23. A method of producing the specific binding protein according to claim 1 comprising:
contacting a cell comprising a polynucleotide encoding the specific binding protein according to claim 1 and a culture medium; and
incubating the cell in the culture medium under conditions suitable for expression of the polynucleotide.

24. A method of treating a condition selected from the group consisting of cancer, inflammation and an autoimmune disorder comprising administering an effective amount of a specific binding protein according to claim 1 to an organism in need.

25. The method according to claim 24 wherein the organism is a human.

26. A method of ameliorating a symptom of a condition selected from the group consisting of cancer, inflammation and an autoimmune disorder comprising administering an effective amount of a specific binding protein according to claim 1 to an organism in need.

27. The method according to claim 26 wherein the organism is a human.

28. A use of a specific binding protein according to claim 1 in the preparation of a medicament for the treatment of a condition selected from the group consisting of cancer, inflammation and an autoimmune disorder.

29. The use according to claim 28 wherein the organism is a human.

30. A use of a specific binding protein according to claim 1 in the preparation of a medicament for ameliorating a symptom of a condition selected from the group consisting of cancer, inflammation and an autoimmune disorder.

31. The use according to claim 30 wherein the organism is a human.
